(19) United States
(12) Patent Application Publication
(10) Pub. No.: US 2026/0000751 A1
(43) Pub. Date: Jan. 1, 2026

(54) CORONAVIRUS VACCINES AND METHODS OF USE THEREOF

(71) Applicants: GEORGIA STATE UNIVERSITY RESEARCH FOUNDATION, INC., Atlanta, GA (US); REGENTS OF THE UNIVERSITY OF MINNESOTA, Minneapolis, MN (US)

(72) Inventors: Lanying DU, Atlanta, GA (US); Juan SHI, Atlanta, GA (US); Gang WANG, Atlanta, GA (US); Frank Jonathan LEE, Minneapolis, MN (US)

(21) Appl. No.: 18/881,444

(22) PCT Filed: Jul. 6, 2023
(86) PCT No.: PCT/US2023/069692

§ 371 Date: Jan. 6, 2025

Related U.S. Application Data
(60) Provisional application No. 63/358,622, filed on Jul. 6, 2022.
(60) Provisional application No. 63/384,301, filed on Nov. 18, 2022.
(60) Provisional application No. 63/385,252, filed on Nov. 29, 2022.
(60) Provisional application No. 63/484,269, filed on Feb. 10, 2023.

(51) Int. Cl. A61K 39/215; A61K 39/00; A61P 37/04

(52) U.S. Cl. CPC A61K 39/215; A61P 37/04; A61K 2039/53; A61K 2039/541; A61K 2039/545

(57) ABSTRACT

The present disclosure relates to coronavirus vaccines and methods for use thereof.

### CROSS-REFERENCE TO RELATED APPLICATIONS

This PCT application claims priority to, and the benefit of U.S. Provisional Patent Application No. 63/358,622, filed Jul. 6, 2022, U.S. Provisional Patent Application No. 63/384,301, filed Nov. 18, 2022, U.S. Provisional Patent Application No. 63/385,252, filed Nov. 29, 2022, U.S. Provisional Patent Application No. 63/484,269, filed Feb. 10, 2023, which are incorporated by reference herein in their entirety.

### STATEMENT REGARDING FEDERALLY SPONSORED RESEARCH

This invention was made with government support under Grant No. AI139092, AI137472, and AI157975 awarded by the National Institutes of Health. The government has certain rights in the invention.

### REFERENCE TO SEQUENCE LISTING

The sequence listing submitted on Jul. 6, 2023, as an .XML file entitled “10013-098WO0_ST26.xml” created on Jul. 6, 2023, and having a file size of 90,263 bytes is hereby incorporated by reference pursuant to 37 C.F.R. § 1.52(e)(5).

### FIELD

The present disclosure relates to coronavirus vaccines and methods for use thereof.

### BACKGROUND

Coronavirus Disease 2019 (COVID-19), which first emerged in 2019, has resulted in a worldwide pandemic with devastating economic losses and threats to public health. COVID-19 is caused by severe acute respiratory syndrome coronavirus-2 (SARS-CoV-2), one of the three highly pathogenic coronaviruses (CoVs) in the beta-CoV genus of the Coronaviridae family. By late January 2023, SARS-CoV-2 had infected more than 753 million individuals worldwide and caused more than 6.80 million deaths. SARS-CoV-2 mutates rapidly and frequently, with multiple mutations being detected in its spike (S) protein and other proteins. These mutations have resulted in different variants of concern (VOCs).

Global collaborative efforts have been made by pharmaceutical companies, academic laboratories, and government agencies, resulting in rapid development of vaccines to prevent SARS-CoV-2 infection or symptoms. Many of the vaccines are aimed at inducing immune responses to the S protein of SARS-CoV-2. The S protein sequence used in these vaccines originates from the initial virus strain and variant strains (including Omicron-BA1). Some of the vaccines have been approved as emergency use authorization (EUA) in a number of countries, which are successful in reducing COVID-19. However, SARS-CoV-2 VOCs continue to be reported worldwide. More variants are expected to emerge as this virus continues to mutate during its replication process.

Given the emergence of additional SARS-CoV-2 VOCs, there is a need to address the aforementioned problems mentioned above by developing vaccines and delivery methods to effectively prevent the global spread of SARS-CoV-2 and other related viruses. The vaccines, compositions, and methods disclosed herein address these and other needs.

### SUMMARY

The present disclosure provides vaccine and nucleic acid compositions against coronaviruses, such as SARS-CoV-2 and other related viruses. The present disclosure provides methods of inducing immune responses to infections from coronaviruses, such as SARS-CoV-2 and other related viruses. The present disclosure also provides methods of preventing or treating coronavirus infections, including, but not limited to, SARS-CoV-2 and other related viruses.

In one aspect, disclosed herein is a vaccine comprising an epitope from an original SARS-CoV-2 receptor-binding domain (RBD) of a spike (S) protein, wherein the epitope is glycosylated. In one embodiment, the RBD comprises a glycosylation site at residue 519 and/or 521.

In some embodiments, the epitope is from an Alpha, Beta, Gamm, Delta, or Omicron variant of the original SARS-CoV-2. In one embodiment, the Omicron variant is selected from BA1, BA2, BA2.12.1, BA4, BA5, XBB1.5, XBB1.16, XBB1.9.1, XBB1.9.2, XBB2.3, other XBB, CH1.1, or BQ1.1.

In one aspect, disclosed herein is a vaccine comprising an epitope from an original SARS-CoV-2 receptor-binding domain of a spike protein, wherein the epitope is glycosylated and fused to a Fc fragment of a human antibody.

In some embodiments, the vaccine comprises a mucosal vaccine. In some embodiments, the receptor-binding domain comprises a glycosylation site at residues 519 and 521, and other naturally occurring mutations at 371, 376, 405, 408, 452, or 486.

In some embodiments, the epitope is from a Delta variant of the original SARS-CoV-2. In some embodiments, the human antibody comprises IgG.

In another aspect, disclosed herein is a method of inducing an immune response against a SARS-CoV-2 virus, comprising administering the vaccine of any preceding aspect.

In another aspect, disclosed herein is a method of preventing or treating a SARS-CoV-2 infection, comprising administering the vaccine of any preceding aspect.

In one aspect, disclosed herein is a method of preventing or treating a SARS-CoV-2 infection, comprising administering a first SARS-CoV-2 vaccine comprising a first spike (S) protein or a fragment thereof and administering a second SARS-CoV-2 vaccine comprising a second S protein or a fragment thereof, wherein the second S protein or a fragment thereof is from a BA1 S protein or other Omicron S protein (BA5, XBB1.5, XBB1.16, XBB1.91, XBB1.92, XBB2.3, CH1.1, or BQ1.1).

In one embodiment, the first spike (S) protein or a fragment thereof is a wild-type S (WT-S) protein or fragment thereof. In some embodiments, the method produces a strong neutralizing antibody response.

In some embodiments, the method produces a high-titer neutralizing antibody response against both the original SARS-CoV-2 and the Omicron subvariant. In some embodiments, the first SARS-CoV-2 vaccine and the second SARS-CoV-2 vaccine are administered concurrently.

In another aspect, disclosed herein is a method of preventing or treating a SARS-CoV-2 infection, comprising administering a subunit vaccine comprising a first spike (S) protein or a fragment thereof and administering a booster dose comprising a second S protein or a fragment thereof, wherein the second S protein or a fragment thereof is from a BA1 S protein or other Omicron S protein (BA5, XBB1.5, XBB1.16, XBB1.91, XBB1.92, XBB2.3, CH1.1, or BQ1.1).

In some embodiments, the first spike (S) protein or a fragment thereof is a wild-type S (WT-S) protein or fragment thereof. In some embodiments, the method produces a strong neutralizing antibody response. In some embodiments, the method produces a high-titer neutralizing antibody response against both the original SARS-CoV-2 and the Omicron subvariant.

In one aspect, disclosed herein is a method of inducing an immune response against a SARS-CoV-2 virus, comprising administering the vaccine of any preceding aspect.

In one aspect, disclosed herein is a method of preventing or treating a SARS-CoV-2 infection, comprising administering the vaccine of any preceding aspect.

In some embodiments, the vaccine induces a T cell-based immune response. In some embodiments, the vaccine provides protection without inducing an antibody response against the spike (S) protein.

In another aspect, disclosed herein is an mRNA vaccine comprising a ribonucleic acid encoding a SARS-CoV-2 spike (S) protein or fragment thereof, wherein the SARS-CoV-2 S protein or fragment thereof comprises six proline amino acid substitutions and a mutated furin cleavage site and a folding protein.

In some embodiments, the SARS-CoV-2 spike (S) protein is from an Omicron variant of SARS-CoV-2. In some embodiments, the Omicron variant is a BA1 variant, or other Omicron variants (BA5, XBB1.5, XBB1.16, XBB1.91, XBB1.92, XBB2.3, CH1.1, or BQ1.1).

In some embodiments, the folding protein is a foldon protein. In some embodiments, the vaccine further encodes a signal peptide. In one embodiment, the signal peptide is a tissue plasminogen activator (tPA) signal peptide.

In some embodiments, the vaccine further encodes a protein tag. In one embodiment, the protein tag is a polyhistidine tag comprising at least six histidine amino acids.

In some embodiments, the vaccine is encapsulated in a lipid nanoparticle.

In one aspect, disclosed herein is a vaccine comprising a SARS-CoV-2 spike protein or fragment thereof, wherein the SARS-CoV-2 spike protein or fragment thereof is encoded from the ribonucleic acid of any preceding aspect.

In another aspect, disclosed herein is a deoxyribonucleic acid (DNA) encoding the mRNA vaccine of any preceding aspect.

In one aspect, disclosed herein is a method of preventing or treating a SARS-CoV-2 infection in a subject, the method comprising administering to the subject a composition comprising the mRNA vaccine of any preceding aspect.

In some embodiments, the method comprises administering to the subject a first booster comprising an mRNA vaccine encoding a receptor-binding domain (RBD) from an original SARS-CoV-2 spike (S) (WT-S) protein, and administering to the subject a second booster comprising an mRNA vaccine encoding the RBD from the original SARS-CoV-2 S (WT-S) protein.

In some embodiments, the composition, the first booster, and the second booster are administered sequentially. In some embodiments, the method comprises administering the composition on a first week. In some embodiments, the method comprises administering the first booster on a second, third or fourth week. In some embodiments, the method comprises administering the second booster on a seventh or eighth week.

In some embodiments, the SARS-CoV-2 virus is an Alpha, Beta, Gamma, Delta, or Omicron SARS-CoV-2 variant, or subvariants thereof.

In one embodiment, the method induces an immune response against a SARS-CoV-2 virus. In some embodiments, the immune response comprises a high-titer neutralizing antibody response against the original SARS-CoV-2, Alpha, Beta, Gamma, Delta, or Omicron SARS-CoV-2 variants, or subvariants thereof.

In some embodiments, the subject is a mammal. In one embodiment, the subject is a human.

In another aspect, disclosed herein is a vaccine comprising a coronavirus spike (S) protein backbone from a first coronavirus and a coronavirus S protein receptor binding domain (RBD) from a second coronavirus, wherein the first coronavirus is a different coronavirus than the second coronavirus.

In some embodiments, the first coronavirus is SARS-CoV-1 (i.e., SARS-CoV) or MERS-CoV. In some embodiments, the second coronavirus variant is SARS-CoV-2. In some embodiments, the first coronavirus is SARS-CoV-2. In some embodiments, the first coronavirus is an Omicron variant. In some embodiments, the first coronavirus is a BA1 variant. In some embodiments, the second coronavirus is a SARS-CoV-1 variant. In some embodiments, the second coronavirus variant is SARS-CoV-1 or MERS-CoV.

In some embodiments, the first coronavirus is selected from SARS-CoV-2 wild-type (WT), Alpha, Beta, Gamma, Delta, or Omicron (Omicron-BA1, Omicron-BA2, Omicron-BA2.75, Omicron-BA4.6, Omicron-BA5, XBB1.5, XBB1.16, XBB1.9.1, XBB1.9.2, XBB2.3, CH1.1, other XBB, or BQ1.1). In some embodiments, the second coronavirus is selected from SARS-CoV-2 WT, Alpha, Beta, Gamma, Delta, or Omicron (Omicron-BA1, Omicron-BA2, Omicron-BA2.75, Omicron-BA4.6, or Omicron-BA5, XBB1.5, XBB1.16, XBB1.9.1, XBB1.9.2, XBB2.3, CH1.1, other XBB, or BQ1.1).

In one aspect, disclosed herein is a vaccine comprising a SARS-CoV-2 spike (S) protein backbone from a first SARS-CoV-2 variant and a SARS-CoV-2 S protein receptor-binding domain (RBD) from a second SARS-CoV-2 variant, wherein the first SARS-CoV-2 variant is a different variant than the second SARS-CoV-2 variant.

In some embodiments, the first SARS-CoV-2 variant is an Omicron variant. In some embodiments, the second SARS-CoV-2 variant is a Delta variant. In some embodiments, the first SARS-CoV-2 variant is a Delta variant. In some embodiments, the second SARS-CoV-2 variant is an Omicron variant.

In some embodiments, the first SARS-CoV-2 variant is selected from SARS-CoV-2 wild-type (WT), Alpha, Beta, Gamma, Delta, or Omicron (Omicron-BA1, Omicron-BA2, Omicron-BA2.75, Omicron-BA4.6, Omicron-BA5, XBB1.5, XBB1.16, XBB1.9.1, XBB1.9.2, XBB2.3, CH1.1, other XBB, or BQ1.1).

In some embodiments, the second SARS-CoV-2 variant is selected from SARS-CoV-2 wild-type (WT), Alpha, Beta, Gamma, Delta, or Omicron (Omicron-BA1, Omicron-BA2, Omicron-BA2.75, Omicron-BA4.6, Omicron-BA5, XBB1.5, XBB1.16, XBB1.9.1, XBB1.9.2, XBB2.3, CH1.1, other XBB, or BQ1.1).

In one embodiment, the vaccine comprises a S-6P-Delta-RBD (i.e., SARS-CoV-2 Omicron-BA1 variant S protein except for its RBD and a Delta variant RBD) vaccine. In some embodiments, the vaccine is a protein subunit vaccine. In some embodiments, the vaccine is an mRNA vaccine.

In one aspect, disclosed herein is a method of inducing an immune response against a coronavirus, comprising: administering the vaccine of any preceding aspect.

In another aspect, disclosed herein is a method of preventing or treating a coronavirus infection, comprising administering the vaccine of any preceding aspect.

In some embodiments, the method further comprises administering an additional vaccine. In some embodiments, the additional vaccine comprises a SARS-CoV-1, a MERS-CoV, or a SARS-CoV-2-glycan mutant protein vaccine.

### BRIEF DESCRIPTION OF FIGURES

The accompanying figures, which are incorporated in and constitute a part of this specification, illustrate several aspects described below.

FIGS. 1A, 1B, 1C, 1D, 1E, 1F, 1G, and 1H show the schematic map of spike (S) protein of SARS-CoV-2 original strain and Omicron subvariants and characterization of SARS-CoV-2 BA1-S protein. Schematic map of SARS-CoV-2 S protein of the original wild-type (WT-S) strain (FIG. 1A) and Omicron BA1 subvariant (BA1-S) (b). Mutant amino acid residues of Omicron BA1 subvariant are shown in the S1 (including N-terminal domain (NTD) and receptor-binding domain (RBD)) and S2 subunits of S protein, respectively. Mutant amino acid residues in the RBD of Omicron BA2, BA2.12.1, and BA5 are shown (FIG. 1B). FP, fusion peptide. HR1 and HR2, heptad repeat regions 1 and 2. TM, transmembrane domain. CP, cytoplasmic tail. ELISA analysis of binding of Omicron BA1-S protein (BA1-S) or original S protein (WT-S) to human angiotensin-converting enzyme 2 (hACE2) (FIG. 1C), hamster ACE2 (FIG. 1D), and mouse ACE2 (FIG. 1E) proteins, respectively. Control, PBS. Statistical significance between the binding of WT-S and BA1-S proteins to mouse ACE2 protein was analyzed using two-tailed student t test, and * (P<0.05) indicates significant difference. (FIG. 1F) Flow cytometry analysis of binding of BA1-S (blue line) and WT-S (red line) proteins to bat ACE2-expressing 293T cells (bat-ACE2/293T). 293T cells were transiently transfected with bat ACE2 plasmid and incubated with each protein (5 μg/ml) for analysis by flow cytometry. Gray shading, mock-incubated cells. MFI, median fluorescence intensity. ELISA for detection of binding of WT-S and BA1-S proteins to SARS-CoV-2 S-vaccinated human (FIG. 1G) and mouse (FIG. 1H) serum neutralizing antibodies, respectively. The data (in FIGS. 1C-1H) are expressed as mean±standard deviation of the mean (s.e.m) of duplicate to quadruple wells. The experiments were repeated twice, leading to similar results.

FIGS. 2A, 2B, 2C, 2D, 2E, 2F, 2G, 2H, 2I, 2J, 2K, 2L, 2M, 2N, and 2O show the cocktail or prime-boost of SARS-CoV-2 WT-S and BA1-S proteins induced potent and durable neutralizing antibodies against four Omicron subvariants and original SARS-CoV-2. BALB/c and K18-hACE2-Tg mice were immunized with WT-S, BA1-S, WT-S prime and BA-S boost, WT-S and BA-S cocktail, or PBS control in the presence of adjuvants for 3 times at a 3-week interval. Sera were collected 10 days after the second immunization and 10, 30 and 90 days, respectively, after the third immunization for detection of neutralizing antibodies against pseudotyped viruses encoding the S protein of the original SARS-CoV-2 strain and Omicron subvariants. NT₅₀ was expressed as 50% neutralizing antibody titers against pseudovirus infection in 293T cells expressing hACE2 receptor (hACE2/293T). NT₅₀ against pseudotyped SARS-CoV-2 original wild-type strain (FIG. 2A and FIG. 2F), BA1 (FIG. 2B and FIG. 2G), BA2 (FIGS. 2C and 2H), BA2.12.1 (FIGS. 2D and 2I), and BA5 (FIG. 2E and FIG. 2J) 10 days after the third immunization in BALB/c (FIGS. 2A-2E) and K18-hACE2-Tg (FIGS. 2F-2J) mice, respectively. NT₅₀ against the afore-mentioned pseudotyped SARS-CoV-2 original wild-type strain (FIG. 2K), BA1 (FIG. 2L), BA2 (FIG. 2M), BA2.12.1 (FIG. 2N), and BA5 (FIG. 2O) 10 days after the second immunization, as well as 10, 30, and 90 days, respectively, after the third immunization. The data are expressed as mean±s.e.m of quadruple wells from pooled sera of five mice in each group. Statistical significance among different vaccination groups was analyzed using Ordinary one-way ANOVA, and * (P<0.05), ** (P<0.01), and *** (P<0.001) indicate significant difference. The experiments were repeated twice, leading to similar results.

FIGS. 3A, 3B, 3C, 3D, 3E, 3F, 3G, 3H, 3I, and 3J show the cocktail or prime-boost of SARS-CoV-2 WT-S and BA1-S proteins induced potent and durable neutralizing antibodies against other SARS-CoV-2 VOCs and SARS-CoV. BALB/c mouse sera collected 10 days after the second immunization and, 10, 30, and 90 days, respectively, after the third immunization of WT-S, BA1-S, WT-S prime and BA-S boost, WT-S and BA-S cocktail, or PBS control were evaluated for neutralizing antibodies against pseudotyped viruses encoding S protein of SARS-CoV-2 Alpha variant containing 10 amino acid mutations (69-70 deletion, 145 deletion, N501Y, A570D, D614G, P681H, T716I, S982A, and D1118H) in the S protein, Beta/Gamma variants containing K417N/T, E484K, and N501Y mutations in the RBD, and Delta variant containing L452R, T478K, and P681R mutations in the S1 subunit, as well as the original SARS-CoV. NT₅₀ was expressed as 50% neutralizing antibody titers against pseudovirus infection in hACE2/293T cells. NT₅₀ against pseudotyped SARS-CoV-2 Alpha (FIG. 3A), Beta (FIG. 3B), Gamma (FIG. 3C), Delta (FIG. 3D) variants, and SARS-CoV (FIG. 3E) 10 days after the third immunization, as well as against pseudotyped SARS-CoV-2 Alpha (FIG. 3F), Beta (FIG. 3G), Gamma (FIG. 3H), Delta (FIG. 3I) variants, and SARS-CoV (FIG. 3J) 10 days after the second immunization and 10, 30, and 90 days, respectively, after the third immunization. The data are expressed as mean±s.e.m of quadruple wells from pooled sera of five mice in each group. Statistical significance among different vaccination groups was analyzed using Ordinary one-way ANOVA, and * (P<0.05), ** (P<0.01), and *** (P<0.001) indicate significant difference. The experiments were repeated twice, leading to similar results.

FIGS. 4A, 4B, 4C, 4D, 4E, 4F, 4G, and 4H show the cocktail or prime-boost of SARS-CoV-2 WT-S and BA1-S proteins completely protected mice from lethal Delta variant challenge. Above immunized K18-hACE2-Tg mice or control mice injected with PBS plus adjuvants were challenged with SARS-CoV-2 Delta variant (10,000 PFU/mouse) 30 days after the last immunization and observed for survival (FIGS. 4A-4D) and body weight changes (FIGS. 4E-4H) for a period of 14 days after challenge. The data (in FIGS. 4E-4H) are expressed as mean±s.e.m of 5 mice in each group. Statistical significance among different vaccination groups was analyzed using Ordinary one-way ANOVA. * (P<0.05) and ** (P<0.01) indicate significant difference between the WT-S and BA1-S (FIG. 4E), WT-S and WT-S/BA1-S cocktail (FIG. 4F), BA1-S and WT-S prime/BA1-S boost (FIG. 4G), as well as BA1-S and WT-S/BA1-S cocktail (FIG. 4H) groups.

FIGS. 5A, 5B, 5C, 5D, 5E, 5F, and 5G show the introduction of glycan probe and characterization of glycosylated mutant SARS-CoV-2 RBD protein. FIG. 5A shows the crystal structure of SARS-CoV-2 RBD (PDB access code: 6M0J). The core structure is colored in cyan, and the receptor-binding motif (RBM) in magenta. Mutated residue (Asn519) is shown where an N-linked glycan probe was introduced. FIG. 5B shows the receptor-binding affinity of mutant RBD (MU-RBD) subunit vaccine. ELISA was carried out to assess the binding of MU-RBD protein to soluble human ACE2 (hACE2) protein. Prototypic wild-type (WT)-RBD protein was included as comparison. FIGS. 5C-5G shows the antibody-binding affinity of MU-RBD subunit vaccine. ELISA was carried out to detect the binding of MU-RBD protein to SARS-CoV-2 RBD-specific neutralizing nanobodies Nanosota-1C-Fc (FIG. 5C) and Ty1 (FIG. 5D), neutralizing mAbs CV30 (FIG. 5E) and EY6A (FIG. 5F) and neutralizing human sera (FIG. 5G). WT-RBD was used as comparison. The data (in FIGS. 5B-5G) are presented as mean±s.e.m. of quadruple wells. The experiments were repeated twice, resulting in similar results.

FIG. 6 shows the immunization and challenge schedules. C57BL/6 and hACE2-Tg mice were immunized with the prototypic wild-type (WT)-RBD, mutant RBD (MU-RBD), or PBS control in the presence of adjuvants for three times at a 3-week interval. Sera were collected at 10 days after the second and third immunizations and detected for IgG antibodies and neutralizing antibodies against pseudotyped and authentic SARS-CoV-2 original strain and variants. Immunized hACE2-Tg mice were challenged with SARS-CoV-2 original strain or Delta variant and observed for survival and weight changes for 14 days. Immunized C57BL/6 mice were challenged with a mouse-adapted SARS-CoV-2 variant and evaluated for viral titers in lungs at day 2 after virus challenge.

FIGS. 7A, 7B, 7C, 7D, 7E, 7F, 7G, 7H, 7I, 7J, 7K, and 7L show the glycosylated mutant SARS-CoV-2 RBD protein elicited improved neutralizing antibodies against SARS-CoV-2 Alpha, Beta, Gamma, and Epsilon variants. hACE2-Tg mice were immunized with the prototypic wild-type (WT)-RBD or mutant RBD (MU-RBD) protein and boosted twice at 3 weeks. Mice injected with PBS were included as control. Mouse sera collected 10 days after the third immunization (FIGS. 7A-7E) and 10 days after the second immunization (FIGS. 7F-7J) were assessed for neutralizing activity against infection of pseudoviruses respectively expressing S protein of SARS-CoV-2 original strain and each variant harboring mutation(s) at the indicated amino acid(s). Alpha variant (B.1.1.7 lineage) contains all 10 amino acid mutations (69-70 deletion, 145 deletion, N501Y, A570D, D614G, P681H, T716I, S982A, and D1118H) in the S protein of SARS-CoV-2. Neutralizing activity was expressed as 50% neutralizing antibody titers (NT₅₀) against pseudovirus infection in hACE2/293T cells. The data are presented as mean±s.e.m of quadruple wells from pooled sera of five mice in each group. *** (P<0.001) indicates significant differences between the MU-RBD and WT-RBD groups. The experiments were repeated twice, resulting in similar results. FIGS. 7K-7L show the calculated neutralizing immunogenicity index (NII) values based on the neutralizing antibody titers (NT₅₀) and the following formula ((NT_{50-WT}−NT_{50-MU})/NT_{50-WT}:NT_{50-WT} and NT_{50-MU} represent NT₅₀ induced by the WT-RBD and MU-RBD, respectively).

FIGS. 8A, 8B, 8C, 8D, 8E, 8F, 8G, 8H, 8I, 8J, 8K, 8L, 8M, 8N, 8O, and 8P show the antibody responses induced by glycosylated mutant SARS-CoV-2 RBD protein. C57BL/6 and hACE2-Tg mice were immunized with the prototypic wild-type (WT)-RBD or mutant RBD (MU-RBD) protein, or PBS control as described above, and collected for sera 10 days after the second and third immunizations. ELISA for detection of IgG antibodies specific to the SARS-CoV-2 WT-RBD (FIG. 8A, FIG. 8E, FIG. 8I, and FIG. 8M), Delta-RBD (FIG. 8B, FIG. 8F, FIG. 8J, and FIG. 8N), Omicron BA.1-RBD (FIG. 8C, FIG. 8G, FIG. 8K, and FIG. 8O), and Omicron BA.2-RBD (FIG. 8D, FIG. 8H, FIG. 8L, and FIG. 8P) from sera of C57BL/6 mice (FIGS. 8A-8H) and hACE2-Tg (FIGS. 8I-8P) mice after the second (FIGS. 8A-8D, FIGS. 8I-8L) and third (FIGS. 8E-8H, FIGS. 8M-8P) immunizations. The data are presented as mean±s.e.m. of quadruple wells from pooled sera of five mice in each group. The experiments were repeated twice, resulting in similar results.

FIGS. 9A, 9B, 9C, 9D, 9E, 9F, 9G, 9H, 9I, 9J, 9K, 9L, 9M, 9N, 9O, 9P, 9Q, 9R, 9S, 9T, 9U, 9V, 9W, and 9X show the glycosylated mutant SARS-CoV-2 RBD protein elicited improved neutralizing antibodies against SARS-CoV-2 Delta and Omicron variants. The same sera described in FIG. 8 were detected for neutralizing antibodies against infection of pseudoviruses respectively expressing S protein of SARS-CoV-2 original strain (FIG. 9A, FIG. 9D, FIG. 9G, and FIG. 9J), Delta variant (harboring L452R-T478K-P681R mutations in the S1 region) (FIG. 9B, FIG. 9E, FIG. 9H, and FIG. 9K), and Omicron variant (harboring 38 amino acid mutations in the S protein) (FIG. 9C, FIG. 9F, FIG. 9I, and FIG. 9L). These sera were collected from C57BL/6 (FIGS. 9A-9F) and hACE2-Tg (FIGS. 9G-9L) mice 10 days after the second (FIGS. 9A-9C, FIGS. 9G-9I) and third (FIGS. 9D-9F, FIGS. 9J-9L) immunizations. Sera from C57BL/6 (FIGS. 9M-9O) and hACE2-Tg (FIGS. 9P-9R) mice after the third immunization were also tested for neutralizing antibodies against infection of authentic SARS-CoV-2 original strain (FIG. 9M and FIG. 9P), Delta (FIG. 9N and FIG. 9Q) and Omicron (FIG. 9O and FIG. 9R) variants. Neutralizing activity was calculated as 50% neutralizing antibody titers (NT₅₀) against infection of each pseudotyped or authentic SARS-CoV-2. ** (P<0.01) and *** (P<0.001) indicate significant differences between the MU-RBD and WT-RBD groups. The date (in FIGS. 9A-9R) is presented as mean±s.e.m. of quadruple wells from pooled sera of five mice in each group. The experiments were repeated twice, resulting in similar results. FIGS. 9S-9X show the calculated neutralizing immunogenicity index (NIT) values based on the neutralizing antibody titers (NT₅₀) and the formula described in FIG. 7.

FIGS. 10A, 10B, 10C, 10D, and 10E show the glycosylated mutant SARS-CoV-2 RBD protein induced enhanced protective efficacy against infection of SARS-CoV-2 original strain and Delta variant. 25 days after the third immunization, hACE2-Tg mice (5 mice/group) were challenged with prototype SARS-CoV-2 human strain (2019n-CoV/USA-WA1/2009, 5,000 PFU/mouse), and observed for survival (FIG. 10A) and body weight changes (FIG. 10B) for 14 days post-challenge. In addition, 50 days after the third immunization, hACE2-Tg mice (5 mice/group) were challenged with SARS-CoV-2 Delta variant (10,000 PFU/mouse) and observed for survival (FIG. 10C) and body weight changes (FIG. 10D) for 14 days post-challenge. 50 days after the third immunization, C57BL/6 mice were challenged with a mouse-adapted strain of SARS-CoV-2 (SARS2-N501Y_{MA30}, 5,000 PFU/mouse), and measured for lung viral titers on day 2 post-challenge. The data (in FIG. 10B, FIG. 10D, and FIG. 10E) are presented as mean±s.e.m. of 5 mice in each group. * (P<0.05), ** (P<0.01), and *** (P<0.001) indicate significant differences between the WT-RBD and PBS (cyan), MU-RBD and PBS (red), or WT-RBD and MU-RBD (black) groups.

FIG. 11 shows the virion of the Omicron BA1 variant and its spike (S) protein and receptor-binding domain (RBD).

FIGS. 12A, 12B, 12C, and 12D show the design of Omicron BA1-S mRNA vaccine and detection of its expression. FIG. 12A shows the schematic map of constructed SARS-CoV-2 BA1-S-mRNA. FIG. 12B shows a mRNA encoding the RBD of the original strain of SARS-CoV-2 (RBD-mRNA) was included as comparison. Each synthesized mRNA was encapsulated with lipid nanoparticles (LNPs) to form mRNA-LNPs for delivery. Detection of protein expression of mRNA by flow cytometry analysis. LNP-encapsulated BA1-S-mRNA (FIG. 12C) or RBD-mRNA (FIG. 12D) was incubated with 293T cells at 37° C. for 48 h, and the cells were stained with mouse-anti-His-FITC antibody, followed by analysis of fluorescence signal (red) using flow cytometry. Gray shading indicates blank cells without incubation with mRNA-LNPs. The data are shown as median fluorescence intensity (MFI) and presented as mean±s.e.m. of duplicate wells. The experiments were repeated three times with similar results.

FIGS. 13A, 13B, 13C, 13D, and 13E show the immunization schedules and vaccine-induced IgG antibody responses. FIG. 13A shows the immunization schedules. BALB/c mice were immunized with each mRNA or PBS control, and boosted twice with RBD-mRNA, BA1-S-mRNA, or their combination, at a 3-week interval. Sera were collected 10 days after the last immunization and detected for specific IgG and subtype antibodies and neutralizing antibodies against the pseudotyped and authentic SARS-CoV-2 original strain and respective variants. IgG antibodies specific to the original SARS-CoV-2 wild-type (WT)-spike (S) protein (FIG. 13B), WT-RBD (FIG. 13C), Delta-RBD (FIG. 13D), or BA1-S (FIG. 13E) protein were tested by ELISA using the mouse sera collected above. The ELISA plates were coated with each protein (1 μg/well), and IgG antibody titers are presented as mean±s.e.m. of four wells of pooled sera from five mice in each group (n=5). Statistical significance was performed using Ordinary one-way ANOVA with Dunnett's multiple comparisons test. * and *** indicate P<0.05 and P<0.001, respectively, and demonstrate significant difference among different groups. The experiments were repeated twice with similar results.

FIGS. 14A, 14B, 14C, 14D, 14E, and 14F show the IgG subtype antibodies induced by different vaccination groups. Mouse sera collected 10 days after the last immunization were assessed by ELISA for IgG1, IgG2a, and IgG2b subtype antibodies specific to the wild-type (WT)-RBD (FIGS. 14A-14C) or BA1-S (FIGS. 14D-14F) protein. The ELISA plates were coated with each protein (1 μg/well). The IgG1 (FIG. 14A and FIG. 14D), IgG2a (FIG. 14B and FIG. 14E), and IgG2b (FIG. 14C and FIG. 14F) antibody titers are presented as mean±s.e.m. of four wells of pooled sera from five mice in each group (n=5). Statistical significance was performed using Ordinary one-way ANOVA with Dunnett's multiple comparisons test. *, **, and *** indicate P<0.05, P<0.01, P<0.001, respectively, and demonstrate significant difference among different groups. The experiments were repeated twice with similar results.

FIGS. 15A, 15B, 15C, 15D, 15E, 15F, and 15G show the sequential immunization induced enhanced neutralizing antibodies against four SARS-CoV-2 Omicron subvariants. Mouse sera collected 10 days after the last immunization were detected for neutralizing antibodies (nAbs) against pseudotyped and authentic Omicron (B.1.1.529) subvariants. Pseudoviruses encoding the S proteins of Omicron BA2, BA2.12.1, and BA5 contained the respective RBD region and the rest of the BA1 S protein regions. NAbs against the original strain of pseudotyped (FIG. 15A) and authentic (FIG. 15B) SARS-CoV-2 wild-type (WT) were included as controls. 50% neutralizing antibody titers (NT₅₀) was calculated based on the antibody's ability to neutralize infection of pseudotyped Omicron BA1 (FIG. 15C), BA2 (FIG. 15D), BA2.12.1 (FIG. 15E), and BA5 (FIG. 15F) subvariants in hACE2/293T cells, as well as authentic Omicron BA1 subvariant in Vero E6 cells (FIG. 15G). The titers of nAbs against pseudotyped and live SARS-CoV-2 are presented as mean±s.e.m. of four and duplicate wells, respectively, of pooled sera from five mice in each group (n=5). Statistical significance was performed using Ordinary one-way ANOVA with Dunnett's multiple comparisons test. *, **, and *** indicate P<0.05, P<0.01, P<0.001, respectively, and demonstrate significant difference among different groups. The experiments were repeated twice with similar results.

FIGS. 16A, 16B, 16C, 16D, and 16E shows the sequential immunization induced neutralizing antibodies against other variants of concern of SARS-CoV-2. Same mouse sera (as in FIG. 15) were also detected for neutralizing antibodies (nAbs) against SARS-CoV-2 other variants of concern (VOCs), including pseudotyped Alpha (B.1.1.7) (FIG. 16A), Beta (B.1.351) (FIG. 16B), Gamma (P.1) (FIG. 16C), and Delta (B.1.617.2) (FIG. 16D) variants, as well as authentic Delta variant (FIG. 16E). Pseudoviruses encoding the S protein of Alpha variant contained 10 amino acid mutations or deletions (69-70 deletion, 145 deletion, N501Y, A570D, D614G, P681H, T716I, S982A, and D1118H) as compared with the S protein of the original SARS-CoV-2. Pseudoviruses encoding the S proteins of Beta and Gamma variants contained K417N or K417T, E484K, and N501Y mutations in the RBD, and the S protein of Delta variant contained L452R, T478K, and P681R mutations in the S1 subunit, of the original SARS-CoV-2. 50% neutralizing antibody titers (NT₅₀) was calculated based on antibody's ability to neutralize infection of each pseudotyped virus variant in hACE2/293T cells or authentic Delta variant in Vero E6 cells. The titers of nAbs against pseudotyped and live SARS-CoV-2 are presented as mean±s.e.m. of four and duplicate wells, respectively, of pooled sera from five mice in each group (n=5). Statistical significance was performed using Ordinary one-way ANOVA with Dunnett's multiple comparisons test. * and *** indicate P<0.05 and P<0.001, respectively, and demonstrate significant difference among different groups. The experiments were repeated twice with similar results.

FIGS. 17A, 17B, 17C, 17D, 17E, 17F, 17G, 17H, 17I, and 17J show that the broadly neutralizing antibodies induced by the designed subunit vaccines against multiple VOCs and the original SARS-CoV-2. The purified proteins, including S-6P, S-6P-Delta-RBD, S-6P-BA5-RBD, S-6P-WT-RBD, and S-6P-no-RBD, were respectively I.M. injected into the K18-hACE2 mice (10 μg/mouse) in the presence of Alum and MPL adjuvants. The Cocktail was prepared by combining the S-6P-Delta-RBD and S-6P-BA5-RBD proteins (5 μg/protein; 10 μg/mouse) with the adjuvants. Mice receiving PBS plus adjuvants were included as control. The mice were boosted twice at 3 weeks with the same immunogen and adjuvants. Sera collected 10 days after the last immunization were evaluated for neutralizing antibodies (Abs) against pseudoviruses encoding the spike (S) protein of the original SARS-CoV-2 strain (FIG. 17 A), Alpha (FIG. 17B), Beta (FIG. 17C), Gamma (FIG. 17D) and Delta (FIG. 17E) variants, as well as the Omicron BA1 (FIG. 17F), BA2 (FIG. 17G), BA2.75 (FIG. 17H), BA4.6 (FIG. 17I) and BA5 (FIG. 17J) subvariants. The NT₅₀ is expressed as 50% neutralizing Ab titers against each pseudovirus infection in hACE2/293T cells. The data are presented as the mean±s.e.m of four wells from pooled sera of five mice in each group. * (P<0.05), ** (P<0.01), and *** (P<0.001) designate significant difference among various vaccination groups. The experiments were repeated at least twice, leading to similar results.

FIGS. 18A and 18B show that the designed subunit vaccines protected inmmunized K18-hACE2 mice against SARS-CoV-2 Omicron variant infection. The K18-hACE2 mice immunized with each protein, including S-6P, S-6P-Delta-RBD, S-6P-BA5-RBD, S-6P-WT-RBD, and S-6P-no-RBD, the Cocktail (combination of the S-6P-Delta-RBD and S-6P-BA5-RBD proteins), or PBS control, were challenged with the Omicron-BA1 VOC, and lung tissues were collected two days after viral infection for detection of viral titers (FIG. 18A) and viral replication (FIG. 18B) by plaque assay and qRT-PCR, respectively. The data are presented as the mean±s.e.m of five mice in each group. * (P<0.05) and *** (P<0.001) designate difference among various vaccination groups.

FIGS. 19A, 19B, 19C, 19D, 19E, 19F, 19G, 19H, 19I, 19J, and 19K show that the designed subunit vaccines protected immunized K18-hACE2 mice against SARS-CoV-2 Delta variant infection. The K18-hACE2 mice immunized with each protein, including S-6P, S-6P-Delta-RBD, S-6P-BA5-RBD, S-6P-WT-RBD, and S-6P-no-RBD, the Cocktail (combination of the S-6P-Delta-RBD and S-6P-BA5-RBD proteins), or PBS control, were I.N. challenged with the SARS-CoV-2 Delta or Omicron-BA1 VOCs two weeks after the last immunization, and were observed for weight changes (FIGS. 19A, 19B, 19C, 19D, and 19E) and survival (FIGS. 19F, 19G, 19H, 19I, 19J, and 19K) for 14 days after challenge. (FIGS. 19A, 19B, 19C, 19D, and 19E) Comparison of weight changes between the S-6P-Delta-RBD and other groups, and the data are presented as the mean+s.e.m of five mice in each group.

FIGS. 20A, 20B, 20C, and 20D show the naïve K18-hACE2 mice receiving S-6P-Delta-RBD immune sera were prevented against SARS-CoV-2 infection without showing pathological effects. The naïve K18-hACE2 mice were I.P. administered with the pooled mouse sera (200 μl/mouse) from each vaccination group, including S-6P, S-6P-Delta-RBD, S-6P-BA5-RBD, S-6P-WT-RBD, and S-6P-no-RBD, the Cocktail (combination of the S-6P-Delta-RBD and S-6P-BA5-RBD proteins), or PBS control, and then I.N. challenged with the SARS-CoV-2 Delta variant 6 h later. This was followed by observation of the mouse weight changes for 4 days, as well as evaluation of the viral titers and pathological changes in the lungs 4 days post-challenge. FIG. 20A shows the 50% neutralizing antibody (Ab) titers (NT₅₀) of pooled mouse sera against the SARS-CoV-2 Delta pseudovirus. This was performed using pseudovirus neutralization assay in hACE2/293T cells. FIG. 20B shows the lung viral titers were tested by plaque assay, and are reported as PFU/g of lung tissues. FIG. 20C shows the weight changes of mice for 4 days after viral challenge. FIG. 20D shows the numbers of edema selected from the H&E-stained lung tissue section slides. Edema numbers were scored 0 (none), 1 (<25%), 2 (26-50%), 3 (51-75%), and 4 (>75%) of tissue fields, respectively. * (P<0.05), ** (P<0.01), and *** (P<0.001) designate significant difference among various groups.

FIGS. 21A, 21B, 21C, 21D, 21E, 21F, and 21G show that the designed subunit vaccines protected immunized K18-hACE2 mice against SARS-CoV infection. The K18-hACE2 mice immunized with each protein, including S-6P, S-6P-Delta-RBD, S-6P-BA5-RBD, S-6P-WT-RBD, and S-6P-no-RBD, the Cocktail (combination of S-6P-Delta-RBD and S-6P-BA5-RBD proteins), or PBS control, were I.N. challenged with the SARS-CoV (MA15 strain) two weeks after the last immunization, and observed for overall survival (FIGS. 21A, 21B, 21C, 21D, 21E, and 21F) and weight changes (FIG. 21G) for 13 days after viral challenge. The data (in FIG. 21G) are presented as the mean+s.e.m of the weight changes from five mice in each group.

FIG. 22 shows the sequence alignment of amino acid residues 331-524 of the original SARS-CoV-2 spike (S) protein (original) (SEQ ID NO: 1) aligned with amino acid residues 1-194 of the SARS-CoV-2 S protein of the present disclosure (query) (SEQ ID NO: 8). Residues in bold, underline of the original sequence (519H and 521P) represent amino acids targeted for mutation to asparagine (N) and threonine (T) residues. Residues in bold, underline of the query sequence (189N and 191T) represent amino acids targeted for glycosylation.

FIGS. 23A and 23B show the mouse immunization procedures and viral challenge schedules. FIG. 23A shows the eight groups of K18-hACE2-Tg (C57BL/6 (B6) background) mice were immunized with the Delta-RBD protein, Omicron-S protein, or PBS control via intramuscular (i.m.), i.m.-intranasal (i.n.), or i.n. route for three doses at a 3-week interval. Aluminum (Alum) plus monophosphoryl lipid A (MPL) and Poly(I:C) adjuvants were used for i.m. and i.n. routes, respectively. Sera collected from the last dose were tested for specific IgG antibodies (Abs), subtype Abs, and neutralizing Abs against SARS-CoV-2 original strains and multiple variants. Immunized mice were then challenged with the SARS-CoV-2 Delta variant, and evaluated for protective efficacy via investigation of survival and weight changes. FIG. 23B shows the eight groups of B6 mice were immunized as described above, and detected for mucosal IgA Abs from collected bronchoalveolar lavage (BAL) fluid.

FIGS. 24A, 24B, 24C, 24D, 24E, 24F, 24G, and 24H show the intranasally immunized Delta-RBD protein with or without Omicron-S priming induced systemic IgG and subtype antibodies. Sera were collected from immunized K18-hACE2 mice 10 days after the last immunization, and tested by ELISA for IgG antibodies specific to the Delta-RBD (FIG. 24A) and Omicron-S (FIG. 24B) proteins, IgG1 subtype antibodies specific to the Delta-RBD (FIG. 24C) and Omicron-S (FIG. 24D) proteins, as well as IgG2c subtype antibodies specific to the Delta-RBD (FIG. 24E) and Omicron-S (FIG. 24F) proteins, respectively. IgG/IgG2c ratios were calculated based on the antibodies specific to the Delta-RBD (FIG. 24G) and Omicron-S (FIG. 24H) proteins, respectively. ELISA plates were coated with the respective protein, and the antibody titer was determined based on the detectable endpoint serum dilution. The data are shown as mean±s.e.m. of five mice in each group. Delta-RBD-i.m. or Delta-RBD-i.n., Delta-RBD protein intramuscular or intranasal immunization. Omicron-S-i.m. or Omicron-S-i.n., Omicron-S protein intramuscular or intranasal immunization. S-i.m.+RBD-i.n., i.m. priming with the Omicron-S protein followed by i.n. boosting with the Delta-RBD protein. Controls, PBS with relevant adjuvants injected via i.m., i.m.+i.n., or i.n. route. * (P<0.05), ** (P<0.01), and *** (P<0.001) designate significant differences among different groups. The experiments were repeated twice, and similar data were acquired.

FIGS. 25A, 25B, 25C, 25D, 25E, and 25F show the intranasally immunized Delta-RBD protein with or without Omicron-S priming induced broadly anti-SARS-CoV-2 neutralizing antibodies. Sera from immunized K18-hACE2 mice 10 days after the last immunization were also tested for neutralizing antibodies (Abs) against pseudotyped original SARS-CoV-2 wild-type strain (FIG. 25A), Delta variant (FIG. 25B), and four Omicron subvariants, including BA1 (FIG. 25C), BA2 (FIG. 25D), BA5 (FIG. 25E), and XBB (FIG. 25F). The pseudovirus neutralization assay was performed in 293T cells expressing SARS-CoV-2 receptor, human ACE2 (hACE2-293T), and 50% neutralizing Ab titer was reported as NT₅₀ against respective pseudovirus infection. The data are shown as mean±s.e.m. of five mice in each group. Delta-RBD-i.m. or Delta-RBD-i.n., Delta-RBD protein intramuscular or intranasal immunization. Omicron-S-i.m. or Omicron-S-i.n., Omicron-S protein intramuscular or intranasal immunization. S-i.m.+RBD-i.n., i.m. priming with the Omicron-S protein followed by i.n. boosting with the Delta-RBD protein. Controls, PBS with relevant adjuvants injected via i.m., i.m.+i.n., or i.n. route. * (P<0.05), ** (P<0.01), and *** (P<0.001) designate significant differences among different groups. The experiments were repeated twice, and similar data were acquired.

FIGS. 26A and 26B show the intranasally immunized Delta-RBD or Omicron-S protein induced potent mucosal IgA antibody responses. Immunized B6 mice were collected for bronchoalveolar lavage (BAL) fluid one month after the last immunization, and tested by ELISA for IgA antibodies specific to the Delta-RBD (FIG. 26A) and Omicron-S (FIG. 26B) proteins, respectively. ELISA plates were coated with the respective protein, and the IgA antibody responses were reported as OD450 values. The data are shown as mean±s.e.m. of five mice in each group. Delta-RBD-i.m. or Delta-RBD-i.n., Delta-RBD protein intramuscular or intranasal immunization. Omicron-S-i.m. or Omicron-S-i.n., Omicron-S protein intramuscular or intranasal immunization. S-i.m.+RBD-i.n., i.m. priming with the Omicron-S protein followed by i.n. boosting with the Delta-RBD protein. Controls, PBS with relevant adjuvants injected via i.m., i.m.+i.n., or i.n. route. The experiments were repeated twice, and similar data were acquired.

FIGS. 27A, 27B, 27C, 27D, 27E, 27F, 27G, 27H, 27I, and 27J show the intranasally immunized Delta-RBD protein with or without Omicron-S priming protected mice from challenge with the SARS-CoV-2 Delta variant. One month after the last immunization, K18-hACE2 mice were challenged with a SARS-CoV-2 Delta variant (10⁴ PFU/mouse), and mouse survival (FIGS. 27A, 27B, 27C, 27D, and 27E) and weight changes (FIG. 27F, 27G, 27H, 27I and 27J) were investigated for 14 days post-challenge. The data (in FIGS. 27F, 27G, 27H, 27I, and 27J) are shown as mean±s.e.m. of five mice in each group. Delta-RBD-i.m. or Delta-RBD-i.n., Delta-RBD protein intramuscular or intranasal immunization. Omicron-S-i.m. or Omicron-S-i.n., Omicron-S protein intramuscular or intranasal immunization. S-i.m.+RBD-i.n., i.m. priming with the Omicron-S protein followed by i.n. boosting with the Delta-RBD protein. Controls, PBS with relevant adjuvants injected via i.m., i.m.+i.n., or i.n. route. * (P<0.05) and *** (P<0.001) designate significant differences among Delta-RBD-i.n. and other groups.

FIGS. 28A, 28B, 28C, 28D, 28E, 28F, 28G, and 28H show the universal coronavirus subunit vaccines induced broadly neutralizing antibodies against different coronaviruses. Mice were immunized with the respective protein vaccines, or PBS plus adjuvants (control), and sera from 10 days after the last dose were evaluated for neutralizing antibodies (Abs) against pseudotyped coronaviruses, including SARS-CoV-2 BA5 variant (FIG. 28A), XBB variant (FIG. 28B), BQ1.1 variant (FIG. 28C), BA1 variant (FIG. 28D), and BA2 variant (FIG. 28E), as well as the original SARS-CoV-2 wild-type strain (FIG. 28F), SARS-CoV (FIG. 28G), and MERS-CoV (FIG. 28H). The pseudovirus neutralization assay was carried out in 293T cells expressing human ACE2 (hACE2-293T) (for SARS-CoV-2 and SARS-CoV) and Huh-7 (for MERS-CoV) cells, respectively. NT₅₀ was calculated as 50% neutralizing Ab titer against respective pseudovirus infection. The data are shown as mean z s.e.m. of five mice in each group. * (P<0.05), ** (P<0.01), and *** (P<0.001) represent significant difference among different groups.

FIGS. 29A and 29B show the universal coronavirus subunit vaccines induced protective efficacy against SARS-CoV-2 BA5 infection. Mice immunized with the respective protein vaccines or control (PBS plus adjuvants) were intranasally (I.N.) challenged with SARS-CoV-2 (Omicron BA5 variant), and lungs were collected 2 days post-challenge for measurement of viral titers (FIG. 29A) and viral replication (FIG. 29B) by plaque assay and qRT-PCR, respectively. The viral titers were expressed as plaque-forming unit (PFU)/g of lung tissues. The levels of viral replication were normalized to glyceraldehyde-3-phosphate dehydrogenase (GAPDH), and the results are shown as a ratio to the GAPDH. The data are presented as mean±s.e.m. of five mice in each group. * (P<0.05), ** (P<0.01), and *** (P<0.001) represent significant difference among different groups.

FIG. 30 shows the universal coronavirus subunit vaccines induced protective efficacy against MERS-CoV infection. Mice immunized with the respective protein vaccines or control (PBS plus adjuvants) were transduced with adenovirus 5-human DPP4 (Ad5CMV/hDPP4-myc-flag), and then I.N. challenged with MERS-CoV-2 (EMC2012 strain). Lungs were collected 3 days post-challenge, and measured for viral titers by plaque assay. The viral titers were expressed as PFU/g of lung tissues. * (P<0.05) and *** (P<0.001) represent significant difference among different groups.

FIGS. 31A, 31B, 31C, 31D, 31E, 31F, 31G, and 31H show the evaluation of mRNA vaccine-induced antibody responses and neutralizing antibodies against SARS-CoV-2 and SARS-CoV. BALB/c mice were immunized with each mRNA vaccine or LNP control for 3 times at 3 weeks, and sera collected 10 days after the last vaccine dose were tested by ELISA for IgG antibodies specific to SARS-CoV-2 S (FIG. 31A) or SARS-CoV RBD (FIG. 31B) protein. These sera were also tested by ELISA for IgG1 subtype antibodies specific to SARS-CoV-2 S (FIG. 31C) or SARS-CoV RBD (FIG. 31D) protein, as well as for IgG2a subtype antibodies specific to SARS-CoV-2 S (FIG. 31E) or SARS-CoV RBD (FIG. 31F) antibodies. ELISA plates were pre-coated with SARS-CoV-2 S or SARS-CoV RBD protein, and respective antibody (Ab) titer was reported as mean±s.e.m. of five mice in each group. The aforementioned mouse sera were evaluated for neutralizing antibodies using a pseudovirus neutralization assay expressing respective S protein of the original SARS-CoV-2 (FIG. 31G) and SARS-CoV (Tor2) (FIG. 31H). 50% neutralizing Ab titer (NT₅₀) was calculated, and the data are presented as mean±s.e.m. of five mice in each group.

FIGS. 32A, 32B, 32C, 32D, 32E, and 32F show the evaluation of mRNA vaccine-induced protective efficacy against infection with the SARS-CoV-2 and SARS-CoV. FIG. 32A shows the 40 days after the last immunization, BALB/c mice were I.N. challenged with the SARS-CoV-2 (Omicron BA1 variant, 10⁵ PFU/ml), and lungs were collected two days later to measure viral titers by plaque assay. The viral titers were reported as PFU/g of lung tissues. The data are presented as mean±s.e.m. of five mice in each group. (FIGS. 32B, 32C, 32D, 32E, and 32F) In a separate experiment, immunized mice were I.N. challenged with the SARS-CoV (MA15 strain, 500 PFU/ml), and observed for survival and weight losses for 14 days after challenge. The data (in FIG. 32C and FIG. 32F) are presented as mean±s.e.m. of three (for survived mice in the LNP control group from day 9) to five mice (for mRNA vaccine groups during days 1-14 and LNP control group by day 8) in each group. Significant differences among different groups are shown as ** (P<0.01) and *** (P<0.001).

FIGS. 33A and 33B show the Evaluation of passive protective efficacy of mRNA vaccine-induced mouse serum antibodies. FIG. 33A shows the naïve BALB/c mice were I.P. injected with pooled sera of mice receiving vaccines (SARS2-S-SARS-RBD mRNA or SARS2-S-RBD-del mRNA) or LNP control, I.N. challenged with the SARS-CoV (MA15 strain, 400 PFU/ml) 12 h later, and measured for viral titers in the lungs by plaque assay two days post-challenge. The viral titers were reported as PFU/g of lung tissues. The data are presented as mean f s.e.m. of five mice in each group. Significant differences among different groups are shown as ** (P<0.01) and *** (P<0.001). FIG. 33B shows the plaque reduction neutralization assay was conducted for the above pooled mouse sera from each group against authentic SARS-CoV (MA15 strain) infection. 50% neutralizing antibody (Ab) titer (NT₅₀) was calculated, and the data are presented as mean±s.e.m. of duplicate wells of pooled sera in each group.

### DETAILED DESCRIPTION

The present disclosure provides vaccine and nucleic acid compositions against coronaviruses, such as SARS-CoV-2 and other related viruses. The present disclosure provides methods of inducing immune responses to infections from coronaviruses, such as SARS-CoV-2 and other related viruses. The present disclosure also provides methods of preventing or treating coronavirus infections, including, but not limited to, SARS-CoV-2 and other related viruses.

To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

By “prevent” or other forms of the word, such as “preventing” or “prevention,” is meant to stop a particular event or characteristic, to stabilize or delay the development or progression of a particular event, characteristic, disease, or disorder, or to minimize the chances that a particular event, characteristic, disease, or disorder will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The terms “treat,” “treating,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A “protein”, “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Omithine, and N-Ethylglycine. Typically, the amide linkages of the peptides are formed from an amino group of the backbone of one amino acid and a carboxyl group of the backbone of another amino acid.

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

A “fragment” is a portion of an amino acid sequence which is identical in sequence to but shorter in length than the reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous amino acid residues of a reference polypeptide, respectively. Fragments may be preferentially selected from certain regions of a molecule, for example the N-terminal region and/or the C-terminal region of a polypeptide.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end. The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules.

A “vaccine” refers to a biological preparation that provides active acquired immunity to a particular infectious diseases caused by a virus, bacteria, parasite, or any other microorganisms. Vaccines typically comprise an agent or several agents, also referred to as antigens, resembling the disease-causing microorganism and is often made from weakened or killed forms of the microbe, its toxins, or its surface proteins/peptides. Vaccines are also made to comprise additional components, such as adjuvants, preservatives, and/or stabilizers to boost the immune response, improve safety, and improve vaccine storage.

Nanoparticles, as described herein, can be synthesized, or assembled via any suitable process. Nanoparticles can be assembled in a single step to minimize process variation, or nanoparticles can be assembled in multiple steps. A single step process can include nanoprecipitation and self-assembly.

As used herein, the term “encapsulate” or “encapsulating” refers to a process in which molecules, such as nucleic acids, proteins, and other macromolecules are surrounded or coated by nanoparticles for delivery to a targeted tissue or cell-type.

A “T cell” refers to a type of lymphocyte that is one of the most important white blood cells of the immune system. T cells can be distinguished from other lymphocytes by the presence of a T-cell receptor (TCR) on their cell surface. The immune-mediated cell death function of T cells is carried by two major subtypes: CD8⁺ “killer” T cells and CD4⁺ “helper T cells.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “mRNA” refers to messenger ribonucleic acid, or single stranded molecule of RNA that corresponds to the genetic sequence of a gene and is translated by a ribosome in the process of synthesizing a protein. mRNA is created during the process of transcription, where a gene is converted into a primary transcript mRNA (or pre-mRNA). The primary transcript is further processed through RNA splicing to only contain regions that will encode protein. mRNA can also be targeted for epigenetic modifications, such as methylation, to impact mRNA translation, nuclear retention, nuclear export, processing, and splicing.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA).

As used herein, “wild-type” refers to the genetic and physical characteristics of the typical form of a species as it occurs in nature. A wild-type or wild type characteristic is conceptualized as a product of the standard “normal” allele at a gene locus, in contrast to that produced by a non-standard “mutant” allele.

A “virus” is a microscopic infectious agent that replicates only inside the living cells of an organism. Viruses can infect all life forms, including mammalian and non-mammalian animals, plants, and other microorganisms. A complete virus, also known as a virion, consists of nucleic acid genetic material surrounded by a protective coat of protein called a capsid. Virus can have a lipid envelope derived from the infected host cell membrane. In general, there are five morphological virus types including helical, icosahedral, prolate, enveloped, and complex virus. A virus can either have a DNA or RNA genome, though a vast majority have RNA genomes. Irrespective of the type of nucleic acid genome, a viral genome can be either a single-stranded genome or a double-stranded genome. A viral variant or subvariant are viruses descending from an original virus comprising at least one genetic mutation.

An “epitope” or “antigenic determinant” refers to the part of an antigen, a molecular structure, or foreign particulate that can bind to a specific antibody or T-cell receptor. The presence of antigens or epitopes of antigens within a host can illicit an immune response.

Throughout the present disclosure, the following terms should be understood. 1) “BA1-S protein” or “S-6P protein” which contains spike (S) protein and receptor-binding domain (RBD) region from SARS-CoV-2 Omicron BA1 subvariant; 2) “BA1-S or S-6P (SARS1-RBD or SARS-RBD)” which contains S protein from SARS-CoV-2 Omicron BA1 region except for its RBD, and the RBD was replaced by SARS-CoV-1 RBD; 3) “BA1-S or S-6P (no-RBD)” which contains S protein from SARS-CoV-2 Omicron BA1 region (but its RBD was removed; 4) “BA1-S or S-6P (Delta-RBD)” which contains S protein from SARS-CoV-2 Omicron BA1 region except for its RBD, and the RBD was replaced by SARS-CoV-2 Delta variant RBD; and 5) “BA1-S or S-6P (WT-RBD)” which contains S protein SARS-CoV-2 Omicron BA1 region except for its RBD, and the RBD was replaced by SARS-CoV-2 original strain RBD.

### Vaccine Compositions

The present disclosure provides vaccine compositions against coronaviruses, such as SARS-CoV-2 and other related viruses.

Severe acute respiratory syndrome coronavirus-2 (SARS-CoV-2) comprises four structural proteins, the spike (S) protein, the nucleocapsid (N) protein, the membrane (M) protein, and the envelope (E) protein. The S protein, or surface spike protein, comprises two subunits, S1 and S2. Further, the S1 subunit comprises a receptor-binding domain (RBD), which binds to host receptors to initiate viral entry. The S protein and RBD protein serves as key targets for developments of SARS-CoV-2 vaccines.

In one aspect, disclosed herein is a vaccine comprising an epitope from a SARS-CoV-2 receptor-binding domain (RBD) of a spike (S) protein, wherein the epitope is glycosylated. In one embodiment, the RBD comprises a glycosylation site at residue 519 and/or 521 of SEQ ID NO:15. In some embodiments, the RBD comprises a glycosylation site at residue 519. In some embodiments, the RBD comprises a glycosylation site at residue 521. In some embodiments, the epitope is from an Omicron variant of SARS-CoV-2. The 519 and 521 positions are determined relevant to SEQ ID NO: 15 (GenBank accession no. QHR63250.2). The numbering on the glycosylation site residues can be determined for other S proteins or when using fragments of the S protein. For example, for SEQ ID NO:1 (or SEQ ID NO:8), the RBD comprises a glycosylation site at residue 189 and/or 191.

In some embodiments, the epitope is from an Alpha, Beta, Gamm, Delta, or Omicron variant of the original SARS-CoV-2. In one embodiment, the Omicron variant is selected from BA1, BA2, BA2.12.1, BA4, BA5, XBB1.5, XBB1.16, XBB1.9.1, XBB1.9.2, XBB2.3, other XBB, CH1.1, or BQ1.1. In some embodiments, the Omicron variant is BA1. In some embodiments, the Omicron variant is BA1. In some embodiments, the Omicron variant is BA2. In some embodiments, the Omicron variant is BA2.12.1. In some embodiments, the Omicron variant is BA4. In some embodiments, the Omicron variant is BA5. In some embodiments, the Omicron variant is XBB1.5. In some embodiments, the Omicron variant is XBB1.16. In some embodiments, the Omicron variant is XBB1.9.1. In some embodiments, the Omicron variant is XBB1.9.2. In some embodiments, the Omicron variant is XBB2.3. In some embodiments, the Omicron variant is CH1.1. In some embodiments, the Omicron variant is BQ1.1.

In some embodiments, the epitope is from an Alpha variant. In some embodiments, the Alpha variant is B.1.1.7. In some embodiments, the epitope is from a Beta variant. In some embodiments, the Beta variant is B.1.351. In some embodiments, the epitope is from a Gamma variant. In some embodiments, the Gamma variant is P.1. In some embodiments, the epitope is from a Delta variant. In some embodiments, the Delta variant is B.1.617.2. In some embodiments, the epitope is from an Epsilon variant. In some embodiments, the Epsilon variant is B.1.427. In some embodiments, the Epsilon variant is B.1.429.

In one aspect, disclosed herein is a vaccine comprising an epitope from an original SARS-CoV-2 receptor-binding domain of a spike protein, wherein the epitope is glycosylated and fused to a Fc fragment of a human antibody.

In some embodiments, the vaccine comprises a mucosal vaccine. In some embodiments, the receptor-binding domain comprises a glycosylation site at residues 519 and/or 521, and other naturally occurring mutations 371, 376, 405, 408, 452, or 486.

In some embodiments, the epitope is from a Delta variant of the original SARS-CoV-2. In some embodiments, the human antibody comprises IgG.

In another aspect, disclosed herein is an mRNA vaccine comprising a ribonucleic acid encoding a SARS-CoV-2 spike (S) protein or fragment thereof, wherein the SARS-CoV-2 S protein or fragment thereof comprises six proline amino acid substitutions and a mutated furin cleavage site and a folding protein.

In one embodiment, the vaccine of any preceding aspect encodes a whole SARS-CoV-2 S protein. In one embodiment, the vaccine of any preceding aspect encodes a fragment of the SARS-CoV-2 S protein. In one embodiment, the vaccine of any preceding aspect encodes 30% or more of a whole SARS-CoV-2 S protein. In one embodiment, the vaccine of any preceding aspect encodes 30%, 35%, 40%, 45%, 50%, 55%, 60%, 65%, 70%, 75%, 80%, 85%, 90%, 95%, or 99% of a whole SARS-CoV-2 S protein.

In one embodiment, the SARS-CoV-2 S protein or fragment thereof from any preceding aspect comprises 1, 2, 3, 4, 5, or 6 proline amino acid substitutions. In one embodiment, the SARS-CoV-2 S protein or fragment thereof from any preceding aspect comprises 6 proline amino acid substitutions. In one embodiment, the SARS-CoV-2 S protein or fragment thereof from any preceding aspect comprises a mutated protease cleavage site. In one embodiment, the protease cleavage site is a mutated furin cleavage site.

In some embodiments, the SARS-CoV-2 S protein or fragment thereof from any preceding aspect comprises 1, 2, 3, 4, 5, or 6 proline amino acid substitutions and a mutated protease cleavage site. In some embodiments, the SARS-CoV-2 S protein or fragment thereof from any preceding aspect comprises 1, 2, 3, 4, 5, or 6 proline amino acid substitutions and a mutated furin cleavage site. In some embodiments, the SARS-CoV-2 S protein or fragment thereof from any preceding aspect comprises six proline amino acid substitutions and a mutated protease cleavage site. In some embodiments, the SARS-CoV-2 S protein or fragment thereof from any preceding aspect comprises six proline amino acid substitutions and a mutated furin cleavage site.

In some embodiments, the SARS-CoV-2 S protein or fragment thereof from any preceding aspect comprises at least one HexaPro sequence. In some embodiments, the HexaPro peptide sequence comprises a mutated furin cleavage site and six proline substitutions.

In some embodiments, the SARS-CoV-2 spike (S) protein is from an Omicron variant of SARS-CoV-2. In some embodiments, the Omicron variant is a BA1 variant. In some embodiments, the Omicron variant is BA1. In some embodiments, the Omicron variant is BA2. In some embodiments, the Omicron variant is BA2.12.1. In some embodiments, the Omicron variant is BA4. In some embodiments, the Omicron variant is BA5. In some embodiments, the Omicron variant is XBB1.5. In some embodiments, the Omicron variant is XBB1.16. In some embodiments, the Omicron variant is XBB1.9.1. In some embodiments, the Omicron variant is XBB1.9.2. In some embodiments, the Omicron variant is XBB2.3. In some embodiments, the Omicron variant is CH1.1. In some embodiments, the Omicron variant is BQ1.1.

In some embodiments, the SARS-CoV-2 spike (S) protein is from an Alpha variant. In some embodiments, the Alpha variant is B.1.1.7. In some embodiments, the SARS-CoV-2 S protein is from a Beta variant. In some embodiments, the Beta variant is B.1.351. In some embodiments, the SARS-CoV-2 S protein is from a Gamma variant. In some embodiments, the Gamma variant is P.1. In some embodiments, the SARS-CoV-2 S protein is from a Delta variant. In some embodiments, the Delta variant is B.1.617.2. In some embodiments, the SARS-CoV-2 S protein is from an Epsilon variant. In some embodiments, the Epsilon variant is B.1.427. In some embodiments, the Epsilon variant is B.1.429.

In some embodiments, the folding protein is a foldon protein. In some embodiments, the folding protein is a trimeric foldon protein. In some embodiments, the vaccine further encodes a signal peptide. In one embodiment, the signal peptide is a tissue plasminogen activator (tPA) signal peptide.

In some embodiments, the vaccine further encodes a protein tag. In one embodiment, the protein tag is a polyhistidine tag comprising at least six histidine amino acids. In some embodiments, the protein tag is a polyhistidine tag comprising 6, 7, 8, 9, 10, 11, 12, 13, 14, 15 or more histidine amino acids.

In some embodiments, the vaccine is encapsulated in a lipid nanoparticle. In some embodiments, the vaccine is a subunit vaccine. In some embodiments, the vaccine neutralizes receptor-binding domain (RBD) activity. In some embodiments, the vaccine elicits neutralizing antibodies to prevent RBD entry.

In another aspect, disclosed herein is a vaccine composition comprising a SARS-CoV-2 spike (S) protein, a HexaPro peptide sequence, and a foldon protein sequence.

In another aspect, disclosed herein is an mRNA vaccine composition comprising a ribonucleic acid sequence encoding a SARS-CoV-2 spike (S) protein, a HexaPro peptide sequence, and a foldon protein sequence.

In another aspect, disclosed herein is a vaccine composition comprising a SARS-CoV-2 Omicron BA1 variant spike (Omicron BA1-S) protein, a HexaPro peptide sequence, and a foldon protein sequence.

In another aspect, disclosed herein is an mRNA vaccine composition comprising a ribonucleic acid sequence encoding a SARS-CoV-2 Omicron BA1 variant spike (Omicron BA1-S) protein, a HexaPro peptide sequence, and a foldon protein sequence.

In some embodiments, the vaccine of any preceding aspect is encoded from the ribonucleic acid of any preceding aspect.

In another aspect, disclosed herein is a vaccine comprising a coronavirus spike (S) protein backbone from a first coronavirus and a coronavirus S protein receptor-binding domain (RBD) from a second coronavirus, wherein the first coronavirus is a different coronavirus than the second coronavirus.

In some embodiments, the first coronavirus is SARS-CoV-1 or MERS-CoV. In some embodiments, the second coronavirus variant is SARS-CoV-2. In some embodiments, the first coronavirus is SARS-CoV-2. In some embodiments, the first coronavirus is an Omicron variant. In some embodiments, the first coronavirus is an Omicron BA1 variant. In some embodiments, the second coronavirus is a SARS-CoV-1 variant. In some embodiments, the second coronavirus variant is SARS-CoV-1 or MERS-CoV.

In some embodiments, the first coronavirus is selected from SARS-CoV-2 wild-type (WT), Alpha, Beta, Gamma, Delta, or Omicron (Omicron-BA1, Omicron-BA2, Omicron-BA2.75, Omicron-BA4.6, Omicron-BA5, XBB1.5, XBB1.16, XBB1.9.1, XBB1.9.2, XBB2.3, other XBB, CH1.1, or BQ1.1). In some embodiments, the second coronavirus is selected from SARS-CoV-2 WT, Alpha, Beta, Gamma. Delta, or Omicron (Omicron-BA1, Omicron-BA2, Omicron-BA2.75, Omicron-BA4.6, Omicron-BA5, XBB1.5, XBB1.16, XBB1.9.1, XBB1.9.2, XBB2.3, other XBB, CH1.1, or BQ1.1).

In one aspect, disclosed herein is a vaccine comprising a SARS-CoV-2 spike (S) protein backbone from a first SARS-CoV-2 variant and a SARS-CoV-2 S protein receptor-binding domain (RBD) from a second SARS-CoV-2 variant, wherein the first SARS-CoV-2 variant is a different variant than the second SARS-CoV-2 variant.

In some embodiments, the first SARS-CoV-2 variant is an Omicron variant. In some embodiments, the second SARS-CoV-2 variant is a Delta variant. In some embodiments, the first SARS-CoV-2 variant is a Delta variant. In some embodiments, the second SARS-CoV-2 variant is an Omicron variant.

In some embodiments, the first SARS-CoV-2 variant is selected from SARS-CoV-2 wild-type (WT), Alpha, Beta, Gamma, Delta, or Omicron (Omicron-BA1, Omicron-BA2, Omicron-BA2.75, Omicron-BA4.6, Omicron-BA5, XBB1.5, XBB1.16, XBB1.9.1, XBB1.9.2, XBB2.3, other XBB, CH1.1, or BQ1.1).

In some embodiments, the second SARS-CoV-2 variant is selected from SARS-CoV-2 WT, Alpha, Beta, Gamma, Delta, or Omicron (Omicron-BA1, Omicron-BA2, Omicron-BA2.75, Omicron-BA4.6, Omicron-BA5, XBB1.5, XBB1.16, XBB1.9.1, XBB1.9.2, XBB2.3, other XBB, CH1.1, or BQ1.1).

In some embodiments, the vaccine comprises a BA1-S or S-6P (Delta-RBD) (i.e., SARS-CoV-2 Omicron-BA1 variant S protein except for its RBD and a Delta variant RBD) vaccine. In some embodiments, the vaccine is a protein subunit vaccine. In some embodiments, the vaccine is an mRNA vaccine.

In another aspect, disclosed herein is a deoxyribonucleic acid (DNA) encoding the mRNA vaccine of any preceding aspect.

In some embodiments, the vaccine comprises SEQ ID NO:1, or a fragment thereof. In some embodiments, the vaccine comprises SEQ ID NO:5, or a fragment thereof. In some embodiments, the vaccine comprises SEQ ID NO:8, or a fragment thereof. In some embodiments, the vaccine comprises a sequence that is at least 60% or more (for example, at least 60%, 65%, 70%, 75%, 80%, 85%, 90%, 95%, 99% or more) identical to SEQ ID NO:1, or a fragment thereof. In some embodiments, the vaccine comprises a sequence that is at least 60% or more (for example, at least 60%, 65%, 70%, 75%, 80%, 85%, 90%, 95%, 99% or more) identical to SEQ ID NO:5, or a fragment thereof. In some embodiments, the vaccine comprises a sequence that is at least 60% or more (for example, at least 60%, 65%, 70%, 75%, 80%, 85%, 90%, 95%, 99% or more) identical to SEQ ID NO:8, or a fragment thereof.

In some embodiments, the vaccine comprises a sequence selected from SEQ ID NOs:5, 6, 8, 9, 10, 11, 12, 13, 14, 15, or a fragment thereof. In some embodiments, the vaccine comprises a sequence that is at least 60% or more (for example, at least 60%, 65%, 70%, 75%, 80%, 85%, 90%, 95%, 99% or more) identical to one of SEQ ID NOs:5, 6, 8, 9, 10, 11, 12, 13, 14, 15, or a fragment thereof.

In some embodiments, the vaccine comprises a sequence selected from SEQ ID NOS:2, 7, or a fragment thereof. In some embodiments, the vaccine comprises a sequence that is at least 60% or more (for example, at least 60%, 65%, 70%, 75%, 80%, 85%, 90%, 95%, 99% or more) identical to one of SEQ ID NOs:2, 7, or a fragment thereof.

In one aspect, disclosed herein is a SARS-CoV-2 vaccine comprising a glycosylated RBD of a Delta variant (Delta-RBD). In some embodiments, the SARS-CoV-2 vaccine is a mucosal vaccine.

It should be noted that SEQ ID NO: 8, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO:28, SEQ ID NO: 29, SEQ ID NO: 30, SEQ ID NO: 31, SEQ ID NO: 32, SEQ ID NO: 33, SEQ ID NO: 34, and SEQ ID NO: 35 are mutants of the RBD sequence (SEQ ID NO: 1), which is a fragment of the larger SARS-CoV-2 S protein (SEQ ID NO: 15). Thus, the numbering for a residue of wild-type or mutant RBD can be determined. For example, mutant RBD amino acid residues 452R, 478K, 371F, 376A, 405N, 408S, 452R, and 486V of SEQ ID NO: 15 are glycosylated at sites 519N and/or 521T of SEQ ID NO: 15 or sites 189N and 191T of SEQ ID NO: 8 and SEQ ID NO: 18, respectively.

In some embodiments, the glycosylated Delta-RBD or Omicron-RBD comprises at least one glycosylation site at 519N and/or 521T, and other mutations at 371F/L, 376A, 405N, 408S, 452R, 478K, or 486P/L/S/V. In some embodiments, the glycosylated Delta-RBD or Omicron-RBD comprises at least 60% or more (for example, at least 60%, 65%, 70%, 75%, 80%, 85%, 90%, 95%, 99% or more) identity to SEQ ID NO: 15. In some embodiments, SEQ ID NO: 15, or a variant thereof, comprises at least one glycosylation site at 519N and/or 521T, and other mutations at 371F/L, 376A, 405N, 408S, 452R, 478K, or 486P/L/S/V.

In some embodiments, the glycosylated Delta-RBD or Omicron-RBD comprises at least one glycosylation site at 189N and/or 191T, and other mutations at amino acid residues 41F/L, 46A, 75N, 78S, 122R, 148K, or 156P/L/S/V. In some embodiments, the glycosylated Delta-RBD comprises at least 60% or more (for example, at least 60%, 65%, 70%, 75%, 80%, 85%, 90%, 95%, 99% or more) identity to SEQ ID NO: 8 or SEQ ID NO: 18. In some embodiments, SEQ ID NO 8 or SEQ ID NO: 18, or a variant thereof, comprises at least one glycosylation site at 189N and/or 191T, and other mutations at amino acid residues 41F/L, 46A, 75N, 78S, 122R, 148K, or 156P/L/S/V.

In some embodiments, the vaccine comprises a glycosylated RBD variant selected from Alpha (B.1.1.7)-RBD, Beta (B.1.351)-RBD, Gamma (P.1)-RBD, Omicron-B.1.1.529-RBD, Omicron-XBB.1.9.1-RBD, Omicron-XBB.1.9.2-RBD, Omicron-BA.2-RBD, Omicron-BA.2.75-RBD, Omicron-BA.2.75.2-RBD, Omicron-BA.2.75.3-RBD, CH.1.1-RBD, Omicron-XBB.2.3-RBD, Omicron-BA.4.6-RBD, Omicron-BA5-RBD, Omicron-XBBL.5-RBD, Omicron-XBBL.16-RBD, or BQ.1.1-RBD. In some embodiments, the vaccine comprises at least 60% or more (for example, at least 60%, 65%, 70%, 75%, 80%, 85%, 90%, 95%, 99% or more) sequence identity to SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35.

In some embodiments, the glycosylated SARS-CoV-2 RBD variants of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35 comprise at least one mutation at amino acid residues 501Y, 417T/N, 484K/A, 339D/H, 371L/F, 373P, 375F, 440K, 446S, 477N, 478K, 493R, 496S, 498R, 505H, 346T, 368I, 376A, 405N, 408S, 444T, 445P, 460K, 486P/L/S/V, 490S, 456L, 452R, 368I, and/or 494P. In some embodiments, SEQ ID NO: 15, or a variant thereof, comprises at least one mutation at amino acid residues 501Y, 417T/N, 484K/A, 339D/H, 371L/F, 373P, 375F, 440K, 446S, 477N, 478K, 493R, 496S, 498R, 505H, 346T, 368I, 376A, 405N, 408S, 444T, 445P, 460K, 486P/L/S/V, 490S, 456L, 452R, 368I, and/or 494P.

It should be noted that the glycosylation epitopes, or glycosylation mutations at amino acid positions 519N and/or 521T (or amino acid positions 189N and/or 191T) apply to the SARS-CoV-2 RBD variants described herein. In some embodiments, the glycosylation epitopes at amino acid positions 519N and/or 521T apply to the glycosylated SARS-CoV-2 RBD variants of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35. In some embodiments, the glycosylation epitopes at amino acid positions 189N and/or 191T apply to the glycosylated SARS-CoV-2 RBD variants of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, or SEQ ID NO: 35.

In some embodiments, the glycosylated SARS-CoV-2 RBD variants comprise Delta-RBD, Alpha (B.1.1.7)-RBD, Beta (B.1.351)-RBD, Gamma (P.1)-RBD, Omicron-B.1.1.529-RBD, Omicron-XBB.1.9.1-RBD, Omicron-XBB.1.9.2-RBD, Omicron-BA.2-RBD, Omicron-BA.2.75-RBD, Omicron-BA.2.75.2-RBD, Omicron-BA.2.75.3-RBD, CH.1.1-RBD, Omicron-XBB.2.3-RBD, Omicron-BA.4.6-RBD, Omicron-BA5-RBD, Omicron-XBB1.5-RBD, Omicron-XBB1.16-RBD, or BQ.1.1-RBD.

It should be noted that the amino acid sequences can vary between virus variants, such as for example, that one variant can have an alanine (A) residue at position 100, whereas another variant can have a lysine (K) at the same position. A non-limiting example includes amino acid residues at position 486 of the Omicron-XBB.1.9.1-RBD, Omicron-BA.2.75-RBD, Omicron-BA.2.75.2-RBD, and Omicron-BA.4.6-RBD variants. Omicron-XBB.1.9.1-RBD comprises a proline (P) at amino acid position 486. Omicron-BA.2.75-RBD comprises a leucine (L) at amino acid position 486. Omicron-BA.2.75.2-RBD comprises a serine (S) at amino acid position 486. Omicron-BA.4.6-RBD comprises a valine (V) at amino acid position 486. Thus, the annotation of mutations to each variant at a specific amino acid position is represented as, for example 486P/L/S/V.

In some embodiments, the glycosylated SARS-CoV-2 RBD variants selected from Delta-RBD, Alpha (B.1.1.7)-RBD, Beta (B.1.351)-RBD, Gamma (P.1)-RBD, Omicron-B.1.1.529-RBD, Omicron-XBB.1.9.1-RBD, Omicron-XBB.1.9.2-RBD, Omicron-BA.2-RBD, Omicron-BA.2.75-RBD, Omicron-BA.2.75.2-RBD, Omicron-BA.2.75.3-RBD, CH.1.1-RBD, Omicron-XBB.2.3-RBD, Omicron-BA.4.6-RBD, Omicron-BA5-RBD, Omicron-XBB1.5-RBD, Omicron-XBB1.16-RBD, or BQ.1.1-RBD comprise at least 60% or more (for example, at least 60%, 65%, 70%, 75%, 80%, 85%, 90%, 95%, 99% or more) identity to SEQ ID NO: 8, SEQ ID NO: 15, or SEQ ID NO: 18.

In some embodiments, the glycosylation epitopes at amino acid positions 519N and/or 521T apply to the glycosylated SARS-CoV-2 RBD variants including, but not limited to Delta-RBD, Alpha (B.1.1.7)-RBD, Beta (B.1.351)-RBD. Gamma (P.1)-RBD, Omicron-B.1.1.529-RBD, Omicron-XBB.1.9.1-RBD, Omicron-XBB.1.9.2-RBD, Omicron-BA.2-RBD, Omicron-BA.2.75-RBD, Omicron-BA.2.75.2-RBD, Omicron-BA.2.75.3-RBD, CH.1.1-RBD, Omicron-XBB.2.3-RBD, Omicron-BA.4.6-RBD, Omicron-BA5-RBD, Omicron-XBB1.5-RBD, Omicron-XBB1.16-RBD, or BQ.1.1-RBD. In some embodiments, the glycosylation epitopes at amino acid positions 189N and/or 191T apply to the glycosylated SARS-CoV-2 RBD variants including, but not limited to Delta-RBD, Alpha (B.1.1.7)-RBD, Beta (B.1.351)-RBD, Gamma (P.1)-RBD, Omicron-B.1.1.529-RBD, Omicron-XBB.1.9.1-RBD, Omicron-XBB.1.9.2-RBD, Omicron-BA.2-RBD, Omicron-BA.2.75-RBD, Omicron-BA.2.75.2-RBD, Omicron-BA.2.75.3-RBD, CH.1.1-RBD, Omicron-XBB.2.3-RBD, Omicron-BA.4.6-RBD, Omicron-BA5-RBD, Omicron-XBB1.5-RBD, Omicron-XBB1.16-RBD, or BQ.1.1-RBD.

In some embodiments, the glycosylated SARS-CoV-2 RBD variants of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO:28, SEQ ID NO: 29, SEQ ID NO: 30, SEQ ID NO: 31, SEQ ID NO: 32, SEQ ID NO: 33, SEQ ID NO: 34, or SEQ ID NO: 35 comprise at least one mutation at amino acid residues 171Y, 87T/N, 154K/A, 9D/H, 41L/F, 43P, 45F, 110K, 116S, 147N, 148K, 163R, 166S, 168R, 175H, 16T, 38I, 46A, 75N, 78S, 114T, 115P, 130K, 156P/L/S/V, 160S, 126L, 122R, 38I, and/or 164P.

In some embodiments, SEQ ID NO: 8, SEQ ID NO: 18, or a variant thereof comprises one or more mutations to amino acid residues selected from 171Y, 87T/N, 154K/A, 9D/H, 41L/F, 43P, 45F, 110K, 116S, 147N, 148K, 163R, 166S, 168R, 175H, 16T, 38I, 46A, 75N, 78S, 114T, 115P, 130K, 156P/L/S/V, 160S, 126L, 122R, 38I, and/or 164P.

In one aspect, disclosed herein is a pan-coronavirus subunit vaccine comprising the conserved backbone of SARS-CoV-2 Omicron BA1 variant spike (S) protein of any preceding aspect. A subunit vaccine targeting a “pan-coronavirus” refers to vaccine or subunit vaccine that provide immunity against current and/or future coronaviruses, including but not limited to SARS-CoV-1, SARS-CoV-2, and MERS-CoV. In some embodiments, the conserved backbone comprises a HexaPro sequences and a C-terminal foldon trimeric sequence and His6 tag. In some embodiments, the RBD of any preceding aspect is replaced with a MERS-CoV RBD (Om-S-MERS-RBD). In some embodiments, the RBD of any preceding aspect is replaced with an Omicron BA5-RBD. In some embodiments, the RBD of any preceding aspect is replaced with a SARS-RBD. In some embodiments, the pan-coronavirus subunit vaccine comprises at least 60% or more (for example, at least 60%, 65%, 70%, 75%, 80%, 85%, 90%, 95%, 99% or more) identity to SEQ ID NO: 17.

It should be understood that the vaccine of any preceding aspect can comprise an original SARS-CoV-2 spike (S) protein, a wild-type (WT)-S protein, a SARS-CoV-2 S protein, or fragments thereof. As used herein, an “original SARS-CoV-2 S protein” refers to the naturally occurring spike glycoprotein expressed by the SARS-CoV-2 strain of coronavirus responsible for entry into host cells. As used herein, a “WT-S protein” refers to a codon optimized S protein derived from the original SARS-CoV-2 S protein. As used herein, a “SARS-CoV-2” S protein refers to a variant of the S protein, or fragments thereof, used to formulate the vaccines of any preceding aspect. Thus, in some embodiments, the vaccine of any preceding aspect comprises an original SARS-CoV-2 S protein, or fragments thereof. In some embodiments, the vaccine of any preceding aspect comprises a WT-S protein, or fragments thereof. In some embodiments, the vaccine of any preceding aspect comprises a SARS-CoV-2 S protein, or fragments thereof. In some embodiments, the vaccine of any preceding aspect comprises a MERS-CoV protein, of fragments thereof.

### Methods of Inducing Immune Responses

The present disclosure provides methods of inducing immune responses against infections from SARS-CoV-2 and other related viruses.

In another aspect, disclosed herein is a method of inducing an immune response against a SARS-CoV-2, a SARS-CoV-1, and/or MERS-CoV virus comprising administering the vaccine of any preceding aspect.

In one aspect, disclosed herein is a method of inducing an immune response against a coronavirus infection comprising administering the vaccine of any preceding aspect.

In some embodiments, the vaccine induces a T cell-based immune response. In some embodiments, the vaccine provides protection without inducing an antibody response against the spike protein.

In some embodiments, the immune response is against a SARS-CoV-2 variant. In some embodiments, the SARS-CoV-2 variant is an Omicron variant. In some embodiments, the SARS-CoV-2 variant is an Alpha variant. In some embodiments, the SARS-CoV-2 variant is a Beta variant. In some embodiments, the SARS-CoV-2 variant is a Gamma variant. In some embodiments, the SARS-CoV-2 variant is a Delta variant. In some embodiments, the SARS-CoV-2 variant is an Epsilon variant.

In some embodiments, the Omicron variant is a BA1 variant. In some embodiments, the Omicron variant is BA1. In some embodiments, the Omicron variant is BA2. In some embodiments, the Omicron variant is BA2.12.1. In some embodiments, the Omicron variant is BA4. In some embodiments, the Omicron variant is BA5. In some embodiments, the Omicron variant is XBB1.5. In some embodiments, the Omicron variant is XBB1.16. In some embodiments, the Omicron variant is XBB1.9.1. In some embodiments, the Omicron variant is XBB1.9.2. In some embodiments, the Omicron variant is XBB2.3. In some embodiments, the Omicron variant is CH1.1. In some embodiments, the Omicron variant is BQ1.1.

In some embodiments, the Alpha variant is B.1.1.7. In some embodiments, the Beta variant is B.1.351. In some embodiments, the Gamma variant is P.1. In some embodiments, the Delta variant is B.1.617.2. In some embodiments, the Epsilon variant is B.1.427. In some embodiments, the Epsilon variant is B.1.429.

### Methods of Preventing or Treating Coronavirus Infections

The present disclosure also provides methods of preventing or treating coronavirus infections, including but not limited to SARS-CoV-2 and other related viruses.

In another aspect, disclosed herein is a method of preventing or treating a SARS-CoV-2, a SARS-CoV-1, and/or MERS-CoV infection, comprising administering the vaccine of any preceding aspect.

In one aspect, disclosed herein is a method of preventing or treating a SARS-CoV-2 infection, comprising administering a first SARS-CoV-2 vaccine comprising a first spike (S) protein or a fragment thereof and administering a second SARS-CoV-2 vaccine comprising a second S protein or a fragment thereof, wherein the second S protein or a fragment thereof is from a BA1 spike protein or other Omicron S protein (BA5, XBB1.5, XBB1.16, XBB1.91, XBB1.92, XBB2.3, CH1.1, or BQ1.1).

In one embodiment, the first spike (S) protein or a fragment thereof is a wild-type S (WT-S) protein or fragment thereof. In some embodiments, the method produces a strong neutralizing antibody response.

In some embodiments, the method produces a high-titer neutralizing antibody response against both the original SARS-CoV-2 wild-type (WT) and the Omicron subvariant (BA1). In some embodiments, the method produces a high-titer neutralizing antibody response against both the original SARS-CoV-2 (WT-S) and a SARS-CoV-2 variant including, but not limited to the Alpha, Beta, Gamma. Delta, Epsilon, Omicron variants, and subvariants thereof. In some embodiments, the method produces a high-titer neutralizing antibody response comprising IgA, IgM, or IgG antibodies.

In some embodiments, the first SARS-CoV-2 vaccine and the second SARS-CoV-2 vaccine are administered concurrently.

In another aspect, disclosed herein is a method of preventing or treating a SARS-CoV-2 infection, comprising administering a subunit vaccine comprising a first spike (S) protein or a fragment thereof and administering a booster dose comprising a second S protein or a fragment thereof, wherein the second S protein or a fragment thereof is from a BA1 S protein or other Omicron S protein (BA5, XBB1.5, XBB1.16, XBB1.91, XBB1.92, XBB2.3, CH1.1, or BQ1.1).

In some embodiments, the first spike (S) protein or a fragment thereof is a wild-type S (WT-S) protein or fragment thereof. In some embodiments, the method produces a strong neutralizing antibody response. In some embodiments, the method produces a high-titer neutralizing antibody response to both the original SARS-CoV-2 wild-type (WT) and the Omicron subvariant (BA1). In some embodiments, the method produces a high-titer neutralizing antibody response comprising IgA, IgM, or IgG antibodies.

In one aspect, disclosed herein is a method of preventing or treating a SARS-CoV-2 infection, comprising administering the vaccine of any preceding aspect.

In some embodiments, the vaccine induces a T cell-based immune response. In some embodiments, the vaccine provides protection without inducing an antibody response against the spike protein.

In one aspect, disclosed herein is a method of preventing or treating a SARS-CoV-2 infection in a subject, the method comprising administering to the subject a composition comprising the mRNA vaccine of any preceding aspect. In some embodiments, the method comprises administering to the subject a first booster comprising an mRNA vaccine encoding a receptor-binding domain (RBD) from an original SARS-CoV-2 S (WT-S) protein, and administering to the subject a second booster comprising an mRNA vaccine encoding the RBD from the original SARS-CoV-2 S (WT-S) protein.

In some embodiments, the composition, the first booster, and the second booster are administered sequentially. In some embodiments, the method comprises administering the composition on a first week. In some embodiments, the method comprises administering the first booster on a third or fourth week. In some embodiments, the method comprises administering the second booster on a seventh or eighth week.

In some embodiments, the SARS-CoV-2 virus is an Alpha, Beta, Gamma, Delta, or Omicron SARS-CoV-2 variant, or subvariants thereof.

In one embodiment, the method induces an immune response against a SARS-CoV-2 virus. In some embodiments, the immune response comprises a high-titer neutralizing antibody response to the original SARS-CoV-2 (WT-S), Alpha, Beta, Gamma, Delta, or Omicron SARS-CoV-2 variants, or subvariants thereof. In some embodiments, the method induces an immune response comprising release of IgA, IgM, or IgG antibodies.

In some embodiments, the subject is a mammal. In one embodiment, the subject is a human.

In another aspect, disclosed herein is a method of preventing or treating a coronavirus infection, comprising administering the vaccine of any preceding aspect.

In some embodiments, the method further comprises administering an additional vaccine. In some embodiments, the additional vaccine comprises a SARS-CoV-1, a MERS-CoV, or a SARS-CoV-2-glycan mutant protein vaccine. In some embodiments, the method further comprises administering an additional booster.

### Administration of Vaccine Compositions

The vaccine composition may be administered in such amounts, time, and route deemed necessary in order to achieve the desired result. The exact amount of the vaccine composition will vary from subject to subject, depending on the species, age, and general condition of the subject, the severity of the infection, the particular composition, its mode of administration, its mode of activity, and the like. The vaccine composition is preferably formulated in dosage unit form for ease of administration and uniformity of dosage. The specific therapeutically effective dose level for any particular subject will depend upon a variety of factors including the age, body weight, general health, sex, and diet of the patient; route of administration, and rate of excretion of the specific vaccine composition employed; and like factors well known in the medical arts.

The vaccine composition may be administered by any route. In some embodiments, the composition is administered via a variety of routes, including oral, intravenous, intramuscular, intra-arterial, intramedullary, intrathecal, subcutaneous, intraventricular, transdermal, intradermal, rectal, intraperitoneal, mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the vaccine composition (e.g., its stability in the environment of the respiratory system), the condition of the subject (e.g., whether the subject is able to tolerate nasal, oral, intravenous, or other selected routes of administration), etc.

The exact amount of a vaccine composition required to achieve a therapeutically or prophylactically effective amount will vary from subject to subject, depending on species, age, and general condition of a subject, severity of any side effects, mode of administration, and the like. The amount to be administered to, for example, a child or an adolescent can be determined by a medical practitioner or person skilled in the art and can be lower or the same as that administered to an adult.

The concentration of the vaccine composition can vary widely and will be selected primarily based on activity of the active ingredient(s), body weight and the like in accordance with the particular mode of administration selected and the patient's needs. Concentrations, however, will be selected to provide dosages ranging from about 0.001 mg/kg/day to about 1 mg/kg/day and sometimes higher. It will be appreciated that such dosages may be varied to optimize a therapeutic and/or prophylactic regimen in a particular subject or group of subjects.

In one aspect, disclosed herein is vaccine composition of any preceding aspect and a pharmaceutically acceptable carrier selected from an excipient, a diluent, a salt, a buffer, a stabilizer, a lipid, an emulsion, and a nanoparticle. One or more active agents (such as, for example viral antigens) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N.Y. Wiley-Interscience.

In some embodiments, the vaccine composition can be prepared as a “concentrate”, e.g. in a storage container of a premeasure volume and/or a predetermined amount ready for dilution with a specified volume of water, saline, or other diluent.

In some embodiments, the vaccine composition is administered 1, 2, 3, 4, 5, or more times. In some embodiments, the vaccine composition is administered weekly, monthly, or yearly. In some embodiments, the vaccine composition is administered every week, every 2 weeks, every 3 weeks, every 4 weeks, or more. In some embodiments, the vaccine composition is administered every month, every 2 months, every 3 months, every 4 months, every 5 months, every 6 months, every 7 months, every 8 months, every 9 months, every 10 months, every 11 months, every 12 months, or more. In some embodiments, the vaccine composition is administered every year, every 2 years, every 3 years, every 4 years, every 5 years, or more.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Effective Vaccination Strategy Using SARS-CoV-2 Spike Cocktail Against Omicron and Other Variants of Concern

Severe acute respiratory coronavirus-2 (SARS-CoV-2) Omicron variant harbors more than 30 mutations in its spike (S) protein. Circulating Omicron subvariants, particularly BA5 and other variants of concern (VOCs) show increased resistance to COVID-19 vaccines that target the original S protein, calling for an urgent need for effective vaccines to prevent multiple SARS-CoV-2 VOCs. Here, the neutralizing activity and protection conferred by a BA1-S subunit vaccine was evaluated when combined with, or used as booster doses after, administration of wild-type S protein (WT-S). A WT-S/BA1-S cocktail, or WT-S prime and BA1-S boost, induced significantly higher neutralizing antibodies against pseudotyped Omicron BA1, BA2, BA2.12.1, and BA5 subvariants, and similar or higher neutralizing antibodies against the original SARS-CoV-2, than the WT-S protein alone. The WT-S/BA1-S cocktail also elicited higher or significantly higher neutralizing antibodies than the WT-S-prime-BA1-S boost, WT-S alone, or BA1-S alone against pseudotyped SARS-CoV-2 Alpha, Beta, Gamma, and Delta VOCs, and SARS-CoV, a closely related beta-coronavirus using the same receptor as SARS-CoV-2 for viral entry. By contrast, WT-S or BA1-S alone failed to induce potent neutralizing antibodies against all these viruses. Similar to the WT-S-prime-BA1-S boost, the WT-S/BA1-S cocktail completely protected mice against lethal challenge of a Delta variant with negligible weight loss. Thus, an effective vaccination strategy was identified that elicits potent, broadly, and durable neutralizing antibodies against circulating SARS-CoV-2 Omicron subvariants, other VOCs, original SARS-CoV-2, and SARS-CoV. These results provide useful guidance for developing efficacious vaccines that inhibit current and future SARS-CoV-2 variants to control the COVID-19 pandemic.

SARS-CoV-2, which causes Coronavirus Disease 2019 (COVID-19), has resulted in devastating damage to human health and to the global economy. The genome of SARS-CoV-2 encodes four structural proteins, including spike (S), membrane (M), envelope (E), and nucleocapsid (N) proteins, among which the S protein plays a critical role in viral infection and pathogenesis; thus, it is a major vaccine and therapeutic target. The S protein comprises S1 and S2 subdomains: the receptor-binding domain (RBD) within the S1 subdomain binds to a cellular receptor, angiotensin-converting enzyme 2 (ACE2), after which the S2 domain initiates fusion between the viral and cell membranes to mediate viral entry into host cells. The S protein has a trimeric structure, with three RBD molecules in the up or down positions; only the RBD in the up position can bind the ACE2 receptor.

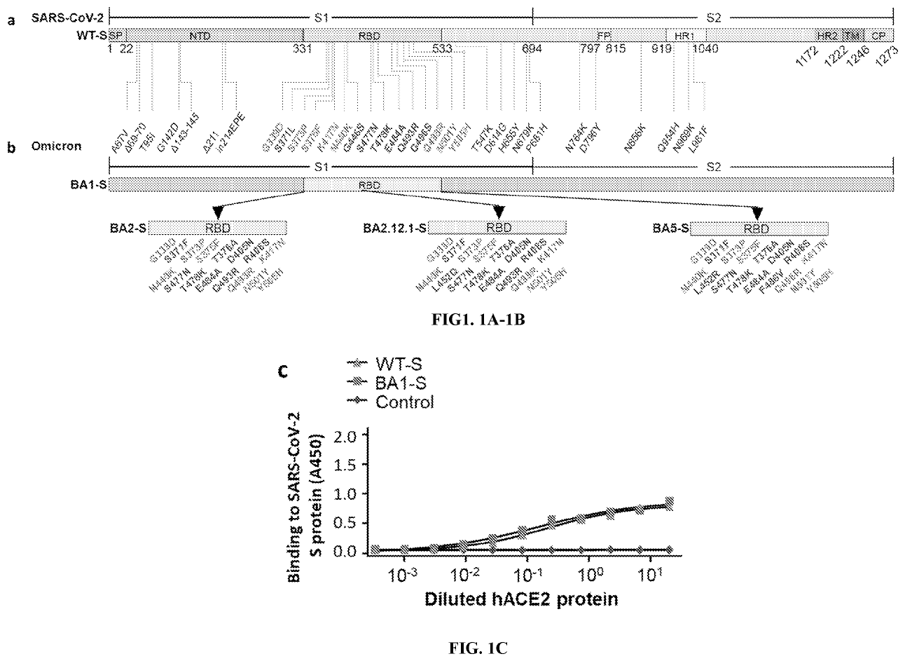

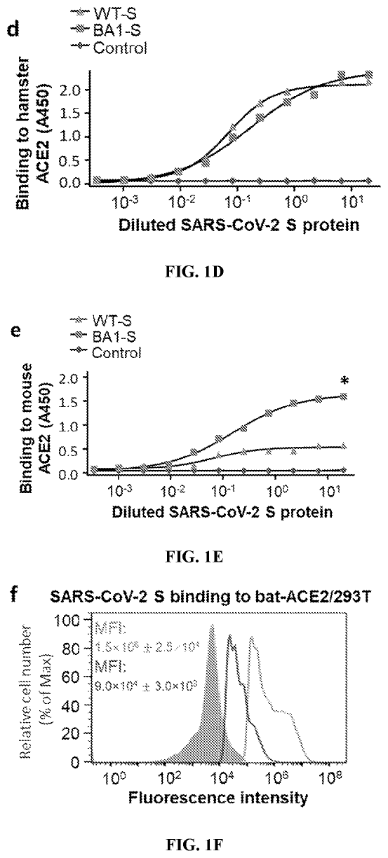

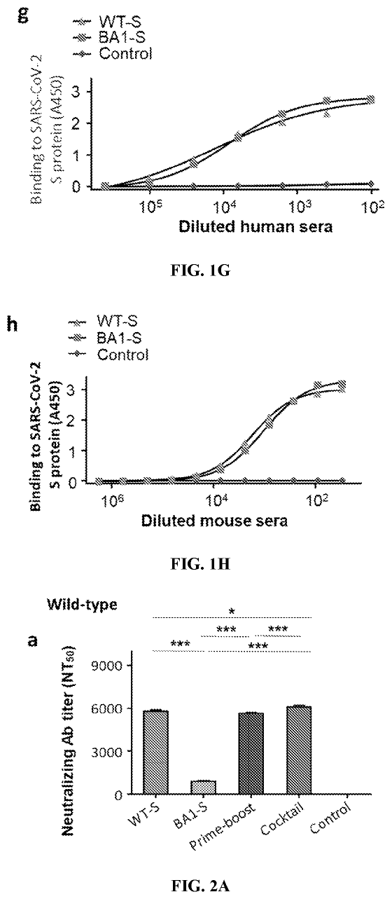

Since the emergence of SARS-CoV-2, a variety of variants of concern (VOCs) have been identified. These variants include previously circulating VOCs Alpha (B.1.1.7), Beta (B.1.351), Gamma (P.1), and Delta (B.1.617.2), and the recently circulating VOC Omicron (B.1.1.529) and its BA.1, BA.2 (BA2.12.1), BA.3, BA.4, and BA.5 subvariants, which have high transmissibility due to many mutations in the S protein. Compared with the original SARS-CoV-2 strain, Omicron BA1 has about 38 amino acid substitutions in the S protein, 15 of which are in the RBD region (FIGS. 1A-1B). Other Omicron subvariants, such as BA2, BA2.12.1, and BA5, also harbor many different amino acid variations in the RBD of the S protein (FIGS. 1A-1B).

Most of these SARS-CoV-2 variants, particularly the currently circulating Omicron subvariants, are resistant to neutralizing antibodies induced by the first-generation COVID-19 vaccines that target the S protein of the original SARS-CoV-2 strain. Thus, there is an urgent need to develop new vaccines to prevent further spread of COVID-19 caused by VOCs, and contain the pandemic. SARS-CoV, a coronavirus belonging to the same beta-coronavirus genus as SARS-CoV-2, caused a global outbreak during 2002-2003; this virus also uses ACE2 as a cellular receptor for vial entry. Here, an effective vaccination strategy was developed using SARS-CoV-2 S protein cocktail that induces potent and durable neutralizing antibody responses against four Omicron subvariants (BA1, BA2, BA2.12.1, and BA5) and other SARS-CoV-2 VOCs (Alpha, Beta, Gamma, and Delta), in addition to the original SARS-CoV-2 and SARS-CoV viruses.

### Results

Characterization of SARS-CoV-2 Omicron BA1-S protein. A recombinant BA1-S expressing the S protein of Omicron BA1 along with HexaPro sequences and a C-terminal foldon sequence (BA1-S), was constructed. In addition, a recombinant WT-S control was constructed to express the S protein of the original SARS-CoV-2 wild-type strain harboring the D614G mutation. Both proteins were expressed in HEK293F cells, purified from cell culture supernatants, and tested for binding to ACE2 receptors from different species. The results showed that the BA1-S protein bound to soluble human ACE2 (hACE2) and hamster ACE2 proteins with binding similar to, or slightly higher than, that of WT-S; however, it bound to mouse ACE2 protein with significantly higher affinity than WT-S (FIGS. 1C-1E). Nevertheless, the binding of BA1-S to cell-associated bat ACE2 was much lower than that of WT-S (FIG. 1F). Similar to WT-S, the BA1-S protein bound efficiently to SARS-CoV-2 vaccinated human and mouse serum neutralizing antibodies (FIGS. 1G-1H). These data show that BA1-S protein binds to the SARS-CoV-2 ACE2 receptor from human, hamster, and mouse, and was antigenic (i.e., it was bound by antibodies in the serum of SARS-CoV-2-vaccinated humans and mice).

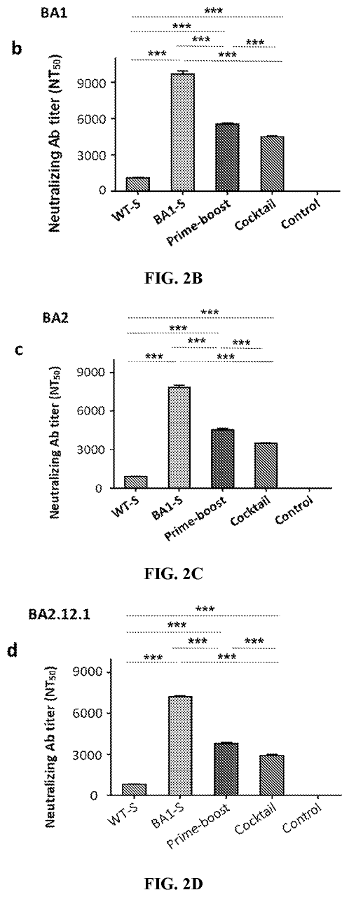

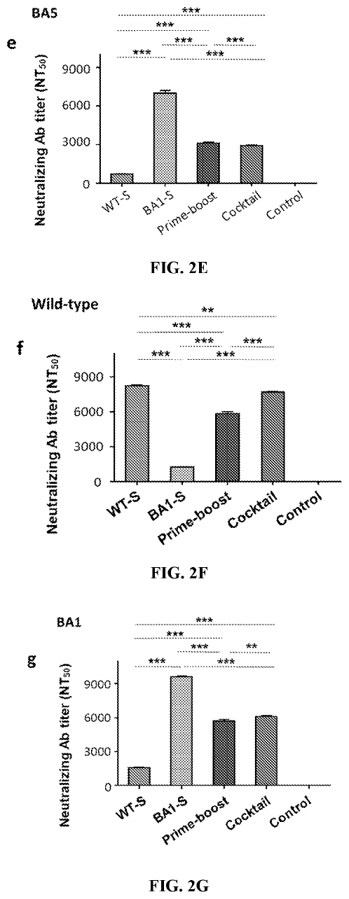

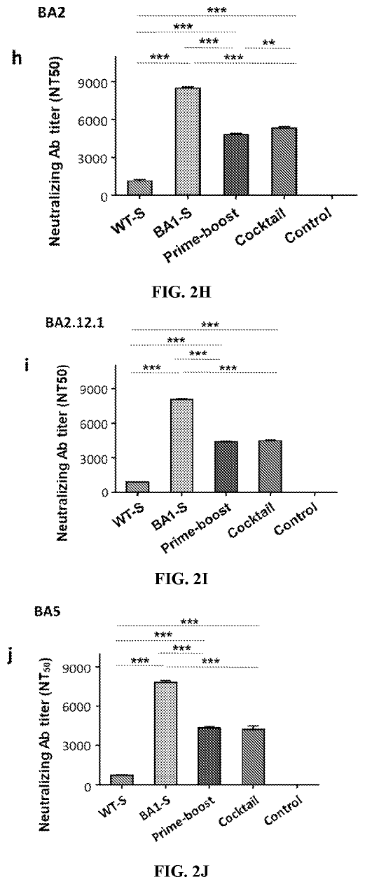

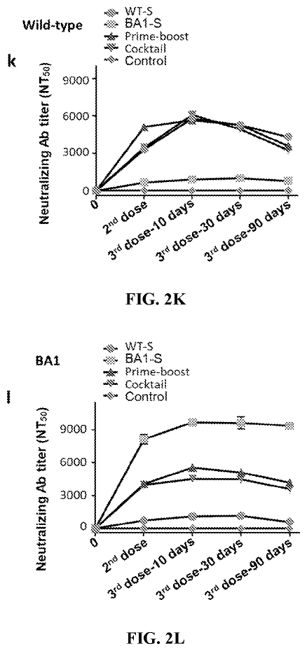

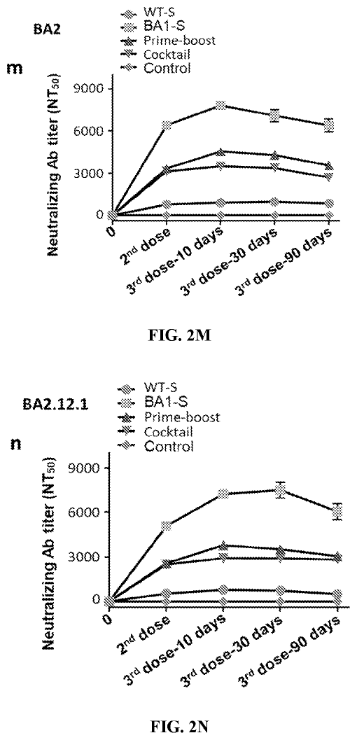

Effective vaccination strategy induced potent and durable neutralizing antibodies against four Omicron subvariants and original SARS-CoV-2. To identify an effective vaccination strategy capable of inducing highly potent neutralizing antibodies against SARS-CoV-2 Omicron subvariants, BALB/c and K18-hACE2-transgenic (Tg) mice were immunized three times with the following proteins (10 μg/mouse) in the presence of adjuvants: 1) WT-S; 2) BA1-S; 3) one dose of WT-S, followed by two doses of BA1-S; and 4) a WT-S/BA1-S cocktail. Mice injected with PBS and adjuvants alone were included as a control. Sera were collected 10 days after the second and third doses, and then 30 and 90 days after the 3^{rd} dose, to evaluate neutralizing activity against pseudotyped viruses expressing the S protein of Omicron subvariants or the original virus. Pseudoviruses encoding the S proteins of BA2, BA2.12.1, and BA5 were constructed by mutation of amino acids in the RBD region (based on the BA1 S protein backbone)(FIGS. 1A-1B). The results from both mouse strains indicated that WT-S or BA1-S alone elicited high-titer neutralizing antibodies against the original SARS-CoV-2 (for WT-S) (FIGS. 2A and 2F) or Omicron subvariants (for BA1-S) (FIGS. 2B-2E and 2G-2J), but not against both the original SARS-CoV-2 and the Omicron subvariants. By contrast, the WT-S prime and BA1-S boost protocol, and the WT-S/BA1-S cocktail, induced significantly higher titers of neutralizing antibodies against the BA1, BA2, BA2.12.1, and BA5 subvariants than the WT-S protein (FIGS. 2B-2E and 2G-2J), as well as similar or higher levels of neutralizing antibodies against the original SARS-CoV-2 strain (FIGS. 2A and 2F). Of note, the WT-S/BA1-S cocktail induced higher or significantly higher neutralizing antibodies against the four Omicron subvariants (for K18-hACE2-Tg mice) (FIGS. 2G-2J) and the original SARS-CoV-2 (for BALB/c and K18-hACE2-Tg mice) (FIGS. 2A and 2F) than the WT-S prime and BA1-S boost protocol. Compared with two doses, a third dose of these vaccines increased the titers of neutralizing antibodies against the original SARS-CoV-2 and all of the Omicron subvariants tested, and these neutralizing antibodies were maintained at high levels for at least 90 days after the third dose (FIGS. 2K-2O). Nevertheless, the PBS control induced background or undetectable levels of neutralizing antibodies against these pseudoviruses (FIG. 2). These data show that unlike the WT-S protein alone, the prime-boost protocol, particularly the WT-S/BA1-S cocktail, induced potent and durable neutralizing antibody titers against all four Omicron subvariants tested, as well as the original SARS-CoV-2 strain, whereas the BA1-S alone only elicited potent neutralizing antibodies against the Omicron subvariants, but not against the original SARS-CoV-2.

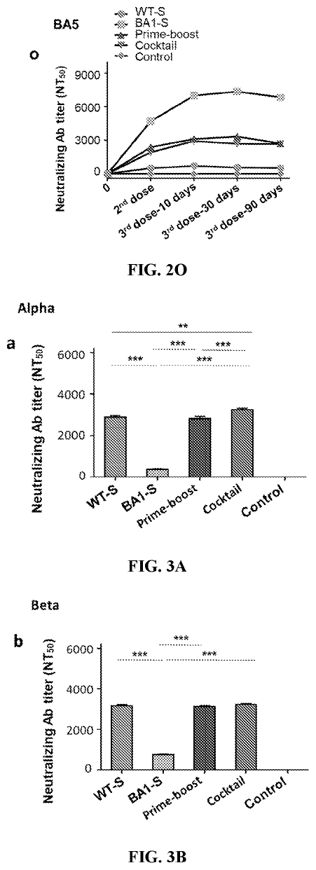

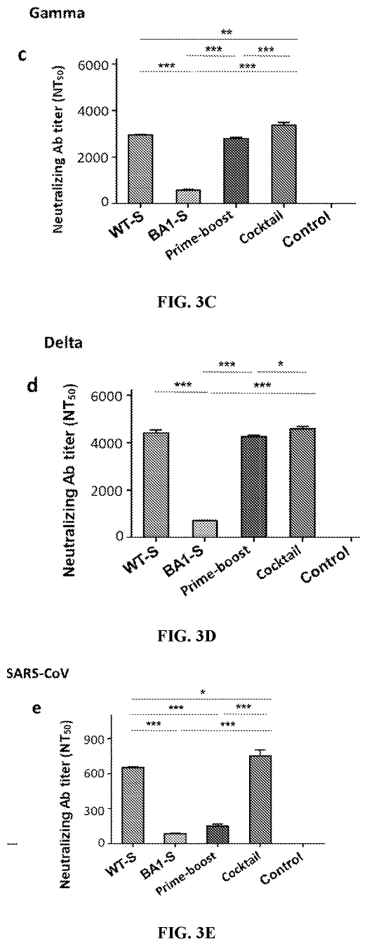

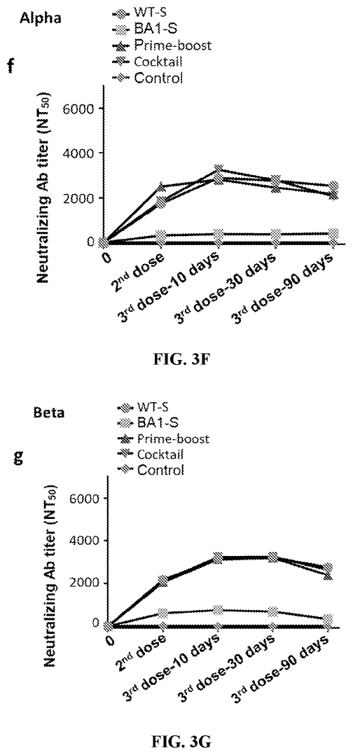

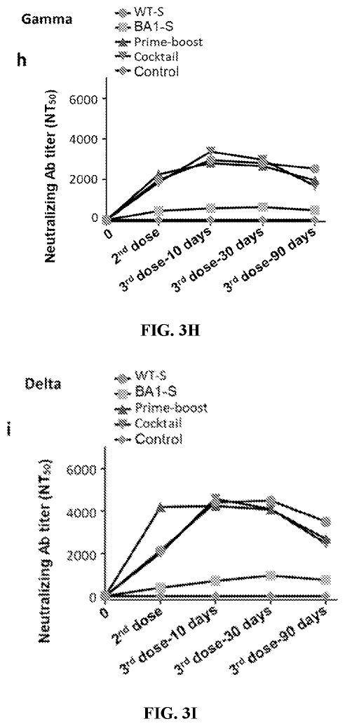

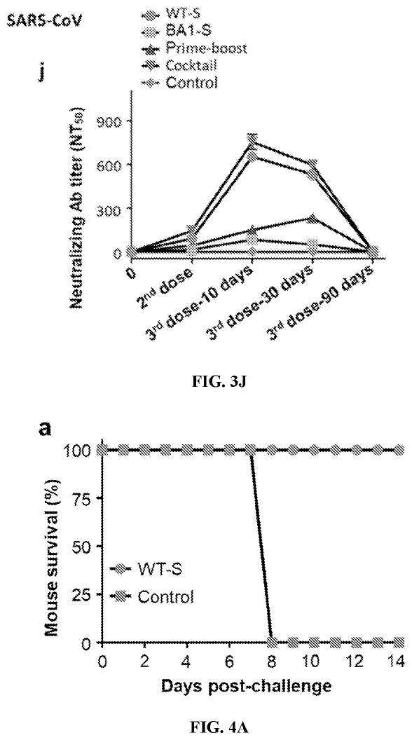

Effective vaccination strategy induced broadly and durable neutralizing antibodies against other SARS-CoV-2 VOCs and SARS-CoV. Sera from vaccinated mice were then tested for their ability to neutralize other SARS-CoV-2 VOCs and SARS-CoV. Pseudoviruses expressing the S protein of the SARS-CoV-2 Alpha, Beta, Gamma, and Delta variants, as well as that of SARS-CoV, were used for the neutralization tests. The results showed that the WT-S/BA1-S cocktail elicited higher or significantly higher neutralizing antibody titers than WT-S alone, or the WT-S prime and BA1-S boost protocol, against the SARS-CoV-2 Alpha, Beta, Gamma, and Delta variants, whereas the WT-S prime and BA1-S boost protocol induced neutralizing antibody titers similar to those of WT-S alone against these viruses (FIGS. 3A-3D). In addition, the WT-S/BA1-S cocktail elicited significantly higher antibody titers than WT-S alone, or the WT-S prime and BA1-S boost protocol, against pseudotyped SARS-CoV (FIG. 3E). The high-titer neutralizing antibodies against these SARS-CoV-2 VOCs were maintained for at least 90 days post-3^{rd} immunization (FIGS. 3F-3I), and against SARS-CoV were maintained for more than 30 days after the third dose (FIG. 3J). Notably, BA1-S elicited the lowest titer of neutralizing antibodies against these viruses, significantly lower than that induced by the other vaccination groups (FIG. 3). By contrast, the PBS control elicited only background or undetectable levels of neutralizing antibodies against these viruses (FIG. 3). The above data shows that unlike the BA1-S protein, the prime-boost, particularly the WT-S/BA1-S cocktail, induce broad and durable neutralizing antibody responses against other SARS-CoV-2 VOCs and SARS-CoV.

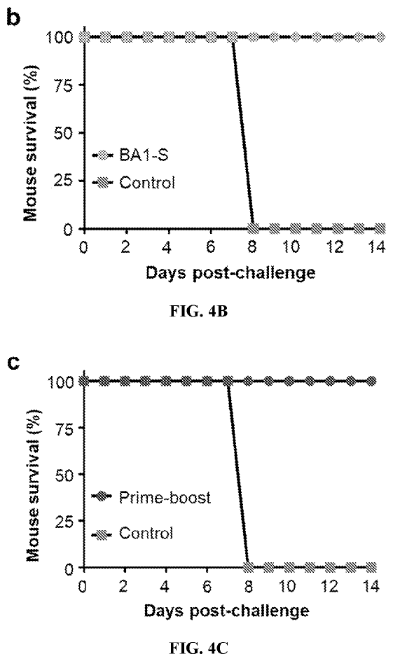

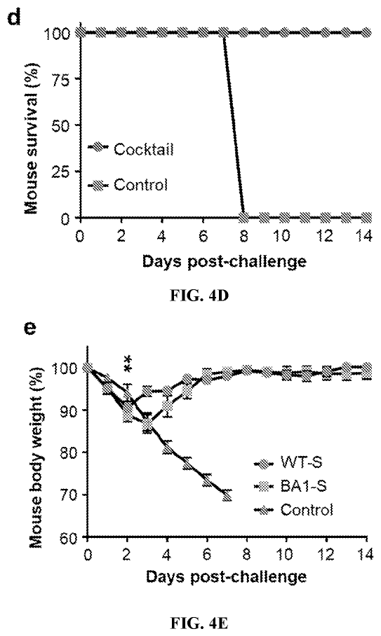

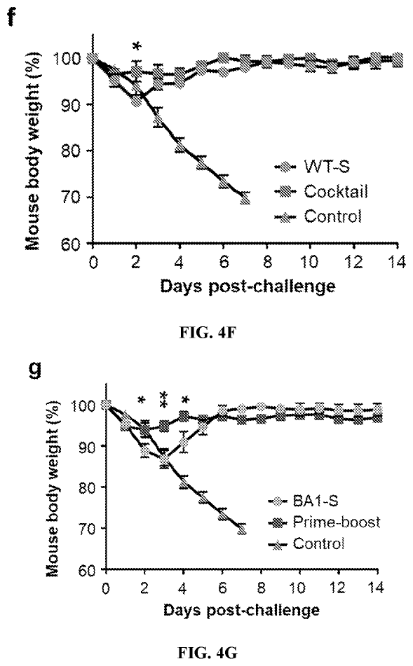

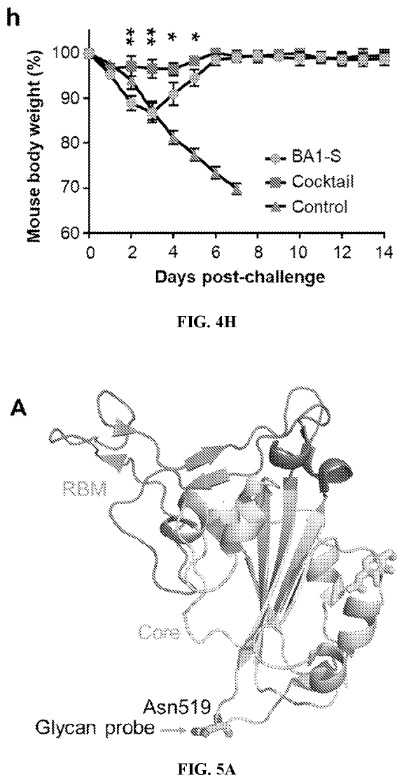

Effective vaccination strategy completely protected mice against Delta variant with significantly less weight loss. Among SARS-CoV-2 variants, the Delta variant shows increased disease severity and mortality. To investigate the protective efficacy of the WT-S/BA1-S or prime-boost vaccination strategy against Delta variant-caused death and weight loss, immunized K18-hACE2-Tg mice were challenged with a lethal dose of SARS-CoV-2 Delta variant 30 days after the last immunization, and survival and weight changes were monitored for 14 days. Mice in the PBS control group showed continuous weight loss, and all died by Day 8 post-virus challenge, whereas all mice in the vaccination groups survived after challenge with the Delta variant (FIGS. 4A-4B). In contrast to significant weight loss (especially on Days 2-5 after challenge) in mice immunized with WT-S or BA1-S alone, the weights of mice immunized using the WT-S/BA1-S cocktail or prime-boost regimens remained unchanged (FIGS. 4E-4H). These data show that similar to the WT-S and BA1-S prime-boost, the WT-S/BA1-S cocktail provided the best protection against weight loss after challenge with the Delta variant.

### Discussion

SARS-CoV-2 Omicron subvariant BA5 is among the recent dominant VOCs circulating in the U.S. and other countries. Although the severity of BA4 and BA5 is similar to that of other Omicron subvariants such as BA1 and BA2, they are more transmissible as current vaccines are less effective against them; increased infections lead inevitably to increased hospitalizations and deaths. The Delta variant also showed higher transmissibility, disease severity, mortality. and hospitalizations than other VOCs such as Alpha and Beta. Most of these VOCs demonstrate resistance to currently available COVID-19 vaccines; this is because they escape immunity induced by these vaccines as they were designed to target the original SARS-CoV-2 strain. Therefore, there is an urgent need to develop effective vaccines capable of inducing balanced immune responses against these VOCs, particularly dominant Omicron BA5, in addition to the original virus strain.

Here, a subunit vaccine was generated based on the S protein of the Omicron BA1 subvariant and evaluated its neutralizing activity; it was evaluated on its own, in combination with the original S protein, and as a booster after vaccination with the original S protein. The Omicron BA1-S protein alone induced high titers of neutralizing antibodies against Omicron subvariants BA1, BA2, BA2.12.1, and BA5, but not against other VOCs or the original SARS-CoV-2. However, the original S protein alone elicited potent neutralizing antibodies against the original SARS-CoV-2, and lower titers against the Alpha, Beta, Gamma, and Delta variants, but it did not induce effective antibodies against Omicron subvariants. By contrast, a WT-S prime and BA1-S boost protocol, particularly the WT-S/BA1-S cocktail, elicited high titers of neutralizing antibodies against all VOCs tested (the four Omicron subvariants, and the Alpha, Beta, Gamma, and Delta variants), as well as the original SARS-CoV-2. In particular, the WT-S/BA1-S cocktail induced more durable and higher titers of antibodies than the WT-S prime and BA1-S boost protocol against the Alpha, Beta, Gamma, and Delta SARS-CoV-2 VOCs, as well as against SARS-CoV.

Of note, the overall titers of neutralizing antibodies elicited by the WT-S prime and BA1-S boost, or WT-S/BA1-S cocktail, against SARS-CoV-2 Omicron subvariants, including the BA5 subvariant, were lower than those against the original SARS-CoV-2 strain. These data are consistent with the reported vaccines, such as those based on mRNAs, viral vectors, and inactivated viruses, which showed reduced neutralizing activity against the Omicron or its subvariants as compared to the original virus isolate or other VOCs. Similarly, skin-patch delivery of a HexaPro S protein of the original SARS-CoV-2 strain elicited higher neutralizing antibody titers against the Gamma and Delta VOCs and original strain, but relatively lower titers of neutralizing antibodies against the Omicron VOC. Previously reported vaccines demonstrated folded reduction of neutralizing antibodies against the Omicron BA4 and BA5 subvariants than against the BA1, BA2, or BA2.12.1 subvariant. In comparison, here it was found that the titers of neutralizing antibodies induced by the WT-S prime and BA1-S boost, or WT-S/BA1-S cocktail, against currently circulating BA5 were not significantly lower than those against the BA1, BA2, and BA2.12.1 subvariants, which were maintained for several months after the last vaccination, showing their potent ability to prevent infection of these Omicron subvariants. Notably, the Omicron BA4 subvariant encodes the same amino acid mutations as BA5 in the S protein RBD region, indicating that vaccine-induced antibodies will also effectively neutralize the BA4 subvariant.

This example also evaluated the in vivo protective efficacy of the above vaccination strategies against infection of the Delta variant due to its increased mortality than other VOCs or the original strain. In either WT-S prime and BA1-S boost or WT-S/BA1-S cocktail protocol, the induced neutralizing antibodies were sufficient to completely protect immunized mice against lethal challenge with the Delta variant, without obvious weight loss.

In conclusion, an effective vaccination strategy was developed to elicit balanced neutralizing antibody responses against currently circulating Omicron subvariants, including BA5, and other SARS-CoV-2 variants. The results serve as useful guidance for development of efficacious vaccines to prevent infection by current and future SARS-CoV-2 variants and control the COVID-19 pandemic.

### Methods

Construction, expression, and purification of recombinant proteins. The DNA-S sequence of SARS-CoV-2 Omicron (B.1.1.529, BA1) variant (BA1-S) was amplified by PCR using a codon-optimized plasmid encoding S protein with HexaPro sequences of SARS-CoV-2 Omicron (GISAID accession number EPI_ISL_6795835). The DNA-S sequence of SARS-CoV-2 wild-type (WT-S) was amplified by PCR using a plasmid encoding codon-optimized S protein of the original SARS-CoV-2 strain (GenBank accession number QHR63250.2). The amplified PCR fragments containing a C-terminal foldon trimeric sequence and His6 tag were inserted into pLenti expression vector. D614G mutation was further added to the WT-S plasmid. The recombinant plasmids were transfected into HEK293F cells, and the related proteins were purified from the culture supernatants using Ni-NTA Superflow (Qiagen).

Enzyme-linked immunoassay (ELISA). ELISA was used to measure the binding between each S protein and ACE2 proteins from different species. For binding to human ACE2 (hACE2) protein, 96-well ELISA plates were coated with purified BA1-S or WT-S protein (1 μg/ml) at 4° C. overnight and blocked with 2% non-fat milk in PBST (PBS containing 0.05% Tween-20) for 1 h at 37° C. The plates were then incubated with serial dilutions of hACE2 protein (Laboratory stock) for 1 h at 37° C. After three washes with PBST, the plates were sequentially incubated with goat anti-hACE2 IgG antibody (0.2 μg/ml, R&D System AF933) and horseradish peroxidase (HRP)-conjugated rabbit anti-goat IgG antibody (1:5,000 dilution, Abcam ab6741). For binding to mouse ACE2 and hamster ACE2, ELISA plates were coated with respective mouse ACE2 (1 μg/ml, R&D System 3437-ZN) or hamster ACE2 (1 μg/ml, R&D System 10578-ZN) protein, and then sequentially incubated with serial dilutions of each S protein, SARS-CoV-2 S-specific mouse polyclonal antibody (1:2,000 dilution, Laboratory stock), and anti-mouse IgG-Fab-HRP antibody (1:5,000 dilution, Sigma A9917-1ML) for 1 h at 37° C. After further washes, the respective plates were incubated with substrate TMB (3,3′,5,5′-Tetramethylbenzidine) (Sigma), and 1 N H₂SO₄ was added to stop the reaction. The A450 value (absorbance at 450 nm) was measured by Cytation 7 Microplate Multi-Mode Reader and Gen5 software (BioTek Instruments).

ELISA was also used to detect the binding between BA1-S or WT-S protein and SARS-CoV-2 vaccinated human or mouse sera. Specifically, ELISA plates were coated with each purified S protein (1 μg/ml), and then sequentially incubated with serial dilutions of human sera from SARS-CoV-2 S-mRNA vaccination or mouse sera from SARS-CoV-2 S-trimer protein immunization. After washes, the plates were incubated with anti-human-IgG-Fab-HRP (1:5,000 dilution, Abcam ab87422) or anti-mouse IgG-Fab-HRP (1:5,000 dilution, Sigma A9917-1ML) antibody for 1 h at 37° C. Other procedures were carried out as described above.

Flow cytometry. Flow cytometry analysis was carried out to detect the binding between BA1-S or WT-S protein and bat ACE2 receptor in bat ACE2-expressing 293T cells as described below. Specifically, 293T cells were transiently transfected with bat ACE2 plasmid using polyetherimide (PEI) method. 48 h later, the transfected cells were incubated with each purified protein (5 μg/ml) for 30 min at room temperature and stained with FITC-conjugated anti-His IgG antibody (1:10 dilution, Invitrogen MA1-81891). After washes and fixing with Fixation/Permeabilization Concentrate reagent (Invitrogen), the cells were analyzed by CytoFLEX flow cytometer (Beckman Coulter Life Sciences), and the data were processed with FlowJo (V10.0).

Construction of recombinant plasmids for package of pseudovirus. Recombinant plasmids respectively encoding S protein of the original SARS-CoV-2 strain (GenBank accession number QHR63250.2) and Alpha (B.1.1.7) variant (GISAID accession number EPI_ISL_718813), as well as the original SARS-CoV strain (GenBank accession number AY274119), were constructed by inserting respective DNA sequences into pcDNA3.1/V5-His-TOPO vector (Thermo Fisher Scientific)³⁸. Omicron (B.1.1.529) variants BA1 (GISAID accession number EPI_ISL_6795835), BA2 (GISAID accession number EPI_ISL_12030355), BA2.12.1 (GISAID accession number EPI_ISL_12061569), and BA5 (GISAID accession number EPI_ISL_12043290), as well as other recombinant plasmids expressing S protein of Beta (B.1.351), Gamma (P.1), and Delta (B.1.617.2) variants, which contain single or multiple amino acid substitutions at the RBD, were constructed using multi-site-directed mutagenesis kit (Agilent Technologies). The recombinant plasmids were confirmed for correct sequences and used for generation of pseudoviruses.

Pseudovirus generation and neutralization assay. Pseudoviruses were generated as described below. Specifically, the plasmid encoding original or mutant S protein was co-transfected with pLenti-CMV-luciferase and PS-PAX2 plasmids (Addgene) into 293T cells using the PEI transfection method described above. Supernatants containing pseudoviruses were collected at 72 h after transfection and processed for below pseudovirus neutralization assay. Pseudoviruses were incubated with serial dilutions of mouse sera for 1 h at 37° C., and the virus-serum mixture was added to 96-well plates pre-seeded with hACE2/293T cells. Fresh medium was added to the cells 24 h later, which were sequentially incubated with cell lysis buffer, and luciferase substrate (Promega) 72 h later. Relative luciferase activity was measured using Cytation 7 Microplate Multi-Mode Reader and Gen5 software (BioTek Instruments). Pseudovirus neutralization was detected, based on which 50% neutralizing antibody titer (NT₅₀) was calculated.

Ethics statement. Female BALB/c mice (4-month-old) mice and K18-hACE2-transgenic (Tg) mice (6-8-week-old) were used in this example. The animal protocols were approved by the Institutional Animal Care and Use Committees (IACUC). All mouse-related experiments were carried out in strict accordance with the Guidelines for the Care and Use of Laboratory Animals of National Institutes of Health and approved protocols.

Mouse immunization and sample collection. Mouse immunization was carried out as described below. Specifically, mice were randomly assigned to each group, and intramuscularly (I.M., 100 μl/mouse) immunized with the following protein or PBS: 1) BA1-S protein, 2) WT-S protein, 3) BA1-S (1^{st} dose) and WT-S (2^{nd}-3^{rd} doses), 4) BA1-S+WT-S cocktail (10 μg/mouse), or 5) PBS control, in the presence of aluminum (500 μg/mouse) and monophosphoryl lipid A (MPL, 10 μg/mouse) adjuvants (InvivoGen). The immunized mice were boosted twice at a 3-week interval, and sera were collected 10 days after the second and third immunizations and 30 and 90 days after the third immunization, respectively, for detection of neutralizing antibodies, as described above.

Challenge of mice with SARS-CoV-2 Delta variant. 30 days after the last immunization (as described above), K18-hACE2-Tg mice were intranasally (I.N.) challenged with SARS-CoV-2 Delta variant (10,000 PFU/mouse, 50 μl/mouse), and observed for survival and body weight changes for up to 14 days after the challenge. Mice with 25% weight loss and significant clinical symptoms, or 30% weight loss, were humanely euthanized by cervical dislocation under anesthesia.

Statistical analysis. Statistical significances among different groups were calculated using GraphPad Prism 9 statistical software. Statistical significance between the binding of each S protein to mouse ACE2 receptor was calculated using two-tailed student t test. Neutralizing antibody titers and weight changes among different groups were calculated using Ordinary one-way ANOVA. P<0.05 was considered as significant. *, **, and *** represent P<0.05, P<0.01, or P<0.001.

### Example 2. A Glycosylated RBD Protein Induces Enhanced Neutralizing Antibodies Against Omicron and Other Variants with Improved Protection Against SARS-CoV-2 Infection

SARS-CoV-2 has mutated frequently since its first emergence in 2019. Numerous variants, is including the currently emerging Omicron variant, have demonstrated high transmissibility or increased disease severity, posting serious threat to global public health. This example describes the identification of an immunodominant non-neutralizing epitope on SARS-CoV-2 receptor-binding domain (RBD). A subunit vaccine against this mutant RBD, constructed by masking this epitope with a glycan probe, did not significantly affect RBD's receptor-binding affinity or antibody-binding affinity, or its ability to induce antibody production. However, this vaccine enhanced the neutralizing activity of this RBD and its protective efficacy in immunized mice. Specifically, this vaccine elicited significantly higher-titer neutralizing antibodies than the prototypic RBD protein against Alpha (B.1.1.7 lineage), Beta (B.1.351 lineage), Gamma (P.1 lineage), and Epsilon (B.1.427 or B.1.429 lineage) variant pseudoviruses containing single or combined mutations in the S protein, albeit the neutralizing antibody titers against some variants were slightly lower than against original SARS-CoV-2. This vaccine also significantly improved the neutralizing activity of the prototypic RBD against pseudotyped and authentic Delta (B.1.617.2 lineage) and Omicron (B.1.1.529 lineage) variants, although the neutralizing antibody titers were lower than against original SARS-CoV-2. In contrast to the prototypic RBD, the mutant RBD completely protected hACE2-transgenic mice from lethal challenge with a prototype SARS-CoV-2 strain and a Delta variant without weight loss. Overall, these findings indicate that this RBD vaccine has broad-spectrum activity against multiple SARS-CoV-2 variants and is an effective vaccine with improved efficacy against Omicron and other pandemic variants.

Several SARS-CoV-2 variants have shown increased transmissibility, calling for a need to develop effective vaccines with broadly neutralizing activity against multiple variants. This example identified a non-neutralizing epitope on the receptor-binding domain (RBD) of SARS-CoV-2 spike protein, and further shielded it with a glycan probe. A subunit vaccine based on this mutant RBD significantly enhanced the ability of prototypic RBD against multiple SARS-CoV-2 variants, including the Delta and Omicron strains, although the neutralizing antibody titers against some of these variants were lower than those against original SARS-CoV-2. This mutant vaccine also enhanced protective efficacy of the prototypic RBD vaccine against SARS-CoV-2 infection in immunized animals. Taken together, this example identified an engineered RBD vaccine against Omicron and other SARS-CoV-2 variants that induced stronger neutralizing antibodies and protection than the original RBD vaccine. It also highlights the need to improve the effectiveness of current COVID-19 vaccines to prevent pandemic SARS-CoV-2 variants.

Coronavirus Disease 2019 (COVID-19), first reported in 2019, has led to a global pandemic with severe economic loss. As of Jul. 19, 2022, more than 559 million COVID-19 cases and at least 6.3 million deaths have been reported worldwide. Severe acute respiratory syndrome coronavirus-2 (SARS-CoV-2), the causative agent of COVID-19, consists of four structural proteins, with the surface spike (S) protein playing a critical role in viral infection and pathogenesis. The S protein consists of two subunits, S1 and S2. The receptor-binding domain (RBD) in S1 subunit binds to a cellular receptor, angiotensin-converting enzyme 2 (ACE2), to initiate the viral entry process, whereas the S2 subunit mediates fusion between the virus and the cell membrane. Therefore, the SARS-CoV-2 S protein and its RBD fragment are key targets for the development of COVID-19 vaccines.

Since its emergence in December 2019, SARS-CoV-2 has undergone continuous mutations, resulting in the occurrence of multiple variants, including Alpha (B.1.1.7 lineage), Beta (B.1.351 lineage), Gamma (P.1 lineage), Delta (B.1.617.2 lineage), and Epsilon (B.1.427 or B.1.429 lineage). A new variant, Omicron (B.1.1.529 lineage), which was first identified in South Africa in late November 2021, has spread rapidly to other countries, accounting for the majority of cases in the U.S. and many countries at present. These variants have been found to include multiple mutations in the S protein or RBD. Thus, it is crucial to understand and rapidly determine whether the current vaccines that target the prototype virus strain are effective against SARS-CoV-2 variants, especially Omicron.

Compared with other vaccine types, such as viral vectored vaccines, subunit vaccines generally have low immunogenicity due to their intrinsic limitations involving the inclusion of various immunodominant non-neutralizing epitopes. Masking of such epitopes by glycan probes or other approaches may focus immune responses against the neutralizing epitopes, thereby potentially increasing the neutralizing capacity of subunit vaccines. A neutralizing immunogenicity index (NII) approach has been developed to calculate the contribution of epitopes to the overall neutralizing immunogenicity of subunit vaccines. This led to the successful design of several mutant subunit vaccines with improved efficacy against the Middle East respiratory syndrome coronavirus (MERS-CoV) and Zika virus.

The present example describes the identification of an immunodominant non-neutralizing epitope containing residue Asn519 of the SARS-CoV-2 S protein, and its masking with a N-linked glycan probe. This enabled the design of a mutant RBD subunit vaccine containing a glycosylation site at residues 519 and 521. The NII of this epitope was determined, as were the receptor-binding affinity and antibody-binding affinity of the resultant mutant RBD, as well as its ability to induce antibody production. This mutant protein was found to improve the neutralizing activity of the RBD, enabling it to neutralize multiple variants of SARS-CoV-2, including Omicron, and to enhance the ability of the RBD to protect animals against SARS-CoV-2 infection.

### Results

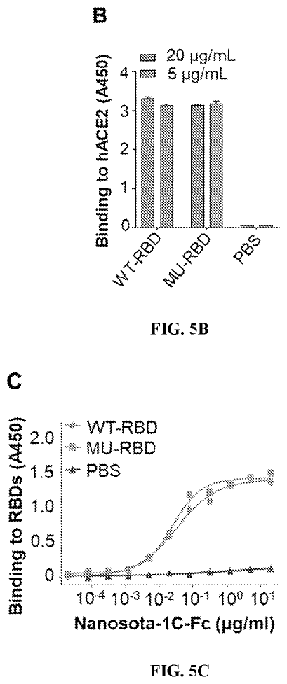

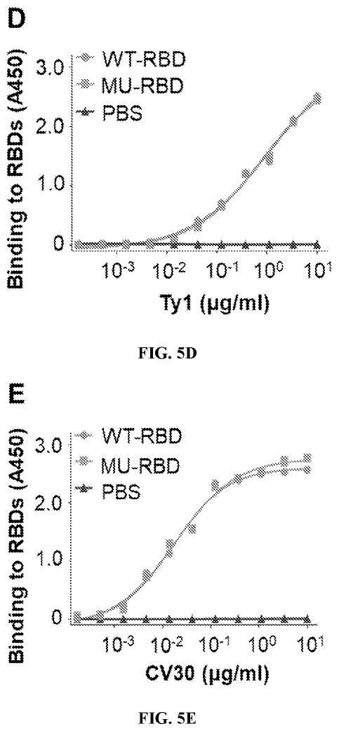

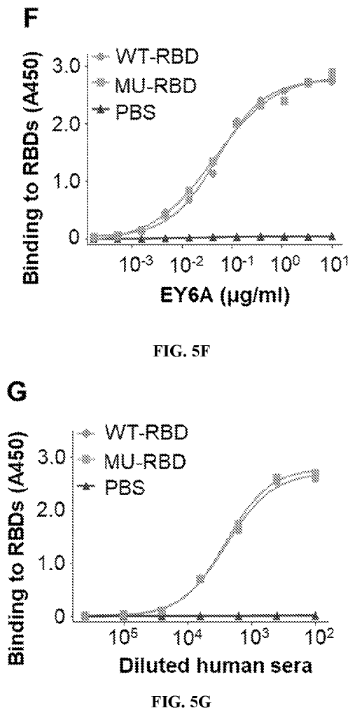

Introduction of a glycan probe onto an epitope and characterization of a glycosylated mutant SARS-CoV-2 RBD protein. A glycan probe was attached to an epitope surrounding residue Asn519 of the RBD of SARS-CoV-2 S protein (FIG. 5A), and mutations were introduced at residues 519 and 521 to generate an N-linked glycosylation site. A recombinant mutant RBD protein was constructed based on this glycosylation site and purified from supernatants of 293T cells transfected with a recombinant plasmid expressing this protein. The binding of the glycosylated mutant RBD protein (MU-RBD) to human ACE2 (hACE2) receptor was similar to that of the prototypic wild-type (WT)-RBD did (FIG. 5B), indicating that masking of this epitope did not affect the ability of the RBD to bind to its receptor. Further analysis revealed that this mutant RBD protein also bound to SARS-CoV-2 RBD-specific neutralizing nanobodies (Nanosota-1C-Fc and Ty1) (FIGS. 5C-5D) and neutralizing monoclonal antibodies (mAbs) (CV30 and EY6A) (FIGS. 5E-5F), as well as serum neutralizing antibodies from COVID-19-vaccinated human (FIG. 5G), similar to the binding of prototypic RBD, showing that the masked epitope did not affect the antigenicity of the RBD or its ability to bind to specific neutralizing antibodies.

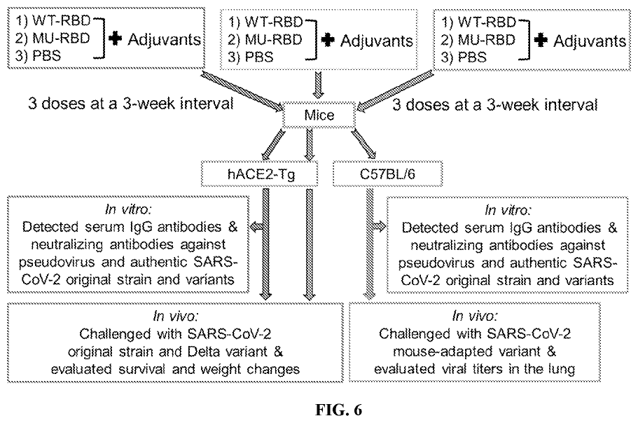

Glycosylated mutant SARS-CoV-2 RBD induced significantly higher-titer neutralizing antibodies than the prototypic RBD against SARS-CoV-2 Alpha, Beta, Gamma, and Epsilon variants. To assess the neutralizing activity of glycosylated mutant RBD against multiple SARS-CoV-2 variants, hACE2-transgenice (Tg) mice were immunized with this protein (FIG. 6), and the titers of the induced neutralizing antibodies against SARS-CoV-2 variants were compared with the titers of antibodies induced by the prototypic RBD. Neutralization analyses were performed using pseudoviruses expressing the S protein of the Alpha, Beta, Gamma, and Epsilon variants of SARS-CoV-2. Mice were immunized three times with prototypic or mutant RBD, and sera of these mice collected 10 days after both the second and the third immunizations were used for the following tests (FIG. 6).

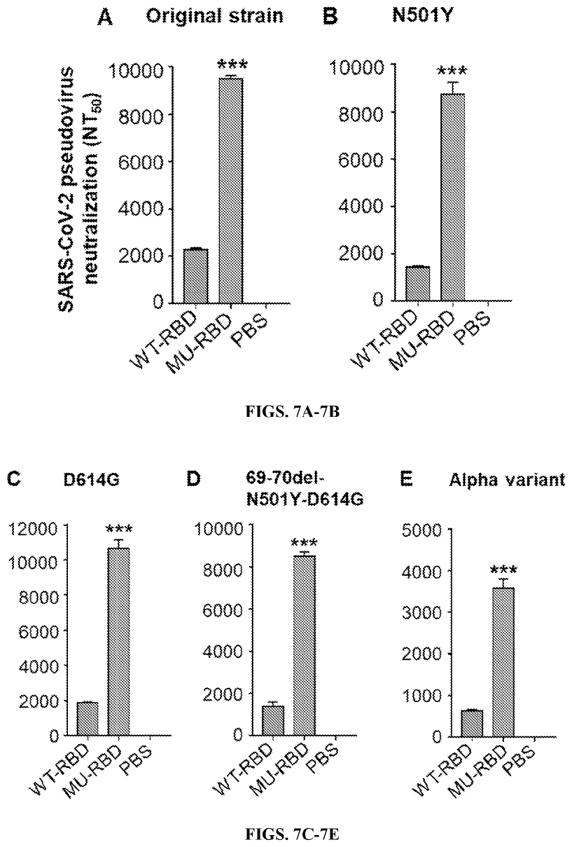

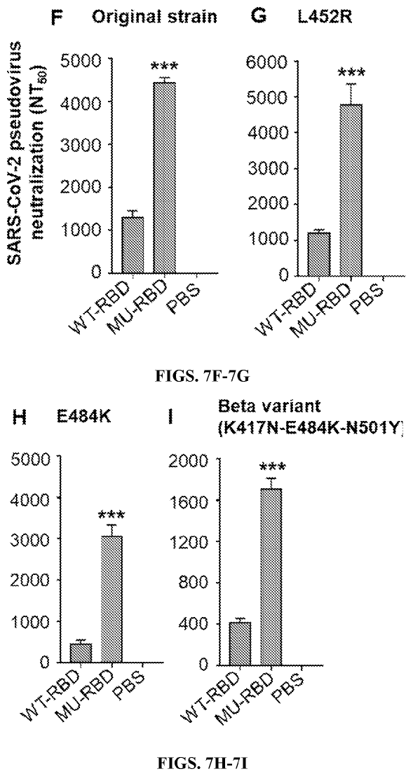

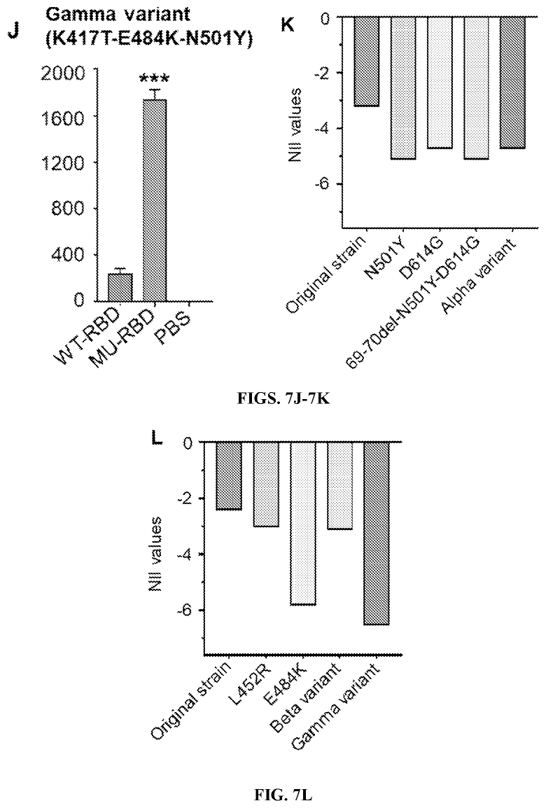

Sera collected from hACE2-Tg mice 10 days after the third immunization demonstrated that, compared with the prototypic RBD, the mutant RBD elicited significantly higher titers of neutralizing antibodies against B.1.1.7 (Alpha) variant pseudoviruses harboring key mutations, including N501Y, D614G, or 67-70del-N501Y-D614G, in the S protein. Moreover, the titers of neutralizing antibodies induced by mutant RBD against pseudovirus containing these mutations were similar to, or slightly different from, those against original SARS-CoV-2 (FIGS. 7A-7D). The titers of the neutralizing antibodies induced by mutant RBD were also significantly higher than those induced by the prototypic RBD protein against an Alpha variant pseudovirus containing all ten amino acid changes in the S protein, although the neutralizing antibody titer induced by mutant RBD was about two-three folds less potent against this variant than against original SARS-CoV-2 strain (FIGS. 7A and 7E).

Sera collected from hACE2-Tg mice 10 days after the second immunization showed that the glycosylated mutant RBD protein induced high-titer neutralizing antibodies against pseudoviruses of Epsilon (B.1.427 or B.1.429 lineage) variants harboring a single L452R mutation, with titers similar to those against original SARS-CoV-2 strain (FIGS. 7F and 7G). These antibodies also efficiently neutralized pseudoviruses bearing a single E484K mutation, or triple mutations of K417N-E484K-N501Y (Beta variant: B.1.351 lineage) and K417T-E484K-N501Y (Gamma variant: P.1 lineage), although the neutralizing antibody titers against the Beta and Gamma variants were lower than those against original SARS-CoV-2 strain (FIGS. 7F and 7H-7J).

The neutralizing immunogenicity index (NII) of an epitope has been defined as the contribution of that epitope to the overall neutralizing immunogenicity of the subunit vaccines. Measurement of the Nil of the glycan-shielded epitope on the RBD showed that its Nils against original SARS-CoV-2 and its variants were all negative (FIGS. 7K and 7L), indicating that this non-neutralizing epitope makes a negative contribution to the overall neutralizing immunogenicity of RBD. Thus, masking the epitope with a glycan probe significantly improved the overall neutralizing activity of the RBD subunit vaccine.

Glycosylated mutant SARS-CoV-2 RBD elicited higher neutralizing antibody titers than the prototypic RBD against SARS-CoV-2 Delta and Omicron variants. The SARS-CoV-2 Delta and Omicron variants have been identified as the variants of concern (VOCs) with high transmissibility and in the case of Delta, disease severity. To investigate the neutralizing activities of glycosylated mutant SARS-CoV-2 RBD against these variants, sera of C57BL/6 and C57BL/6-background hACE2-Tg mice collected 10 days after the second and third immunizations were tested for neutralizing activity against pseudotyped Delta and Omicron variants, which contain L452R-T478K-P681R, and 38 amino acid mutations, respectively, in their S protein, or against authentic Delta and Omicron variants (FIG. 6).

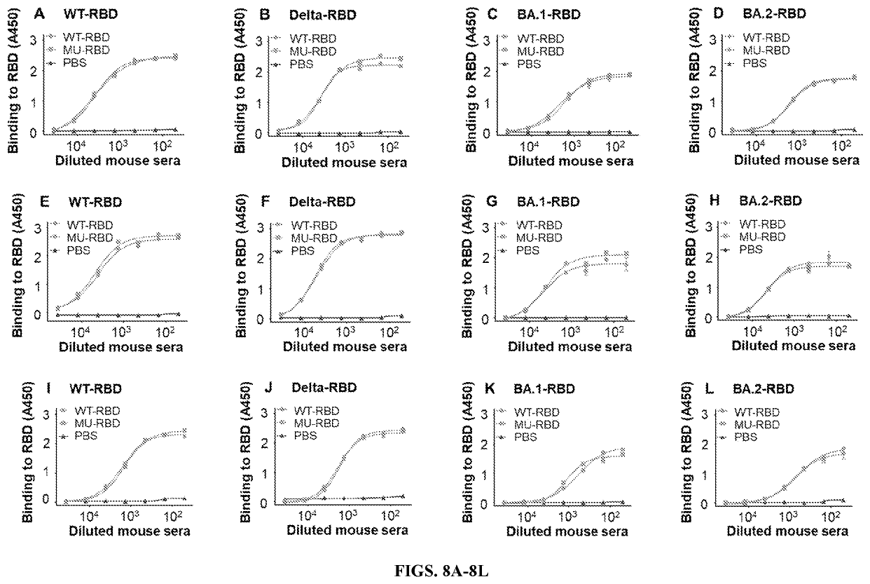

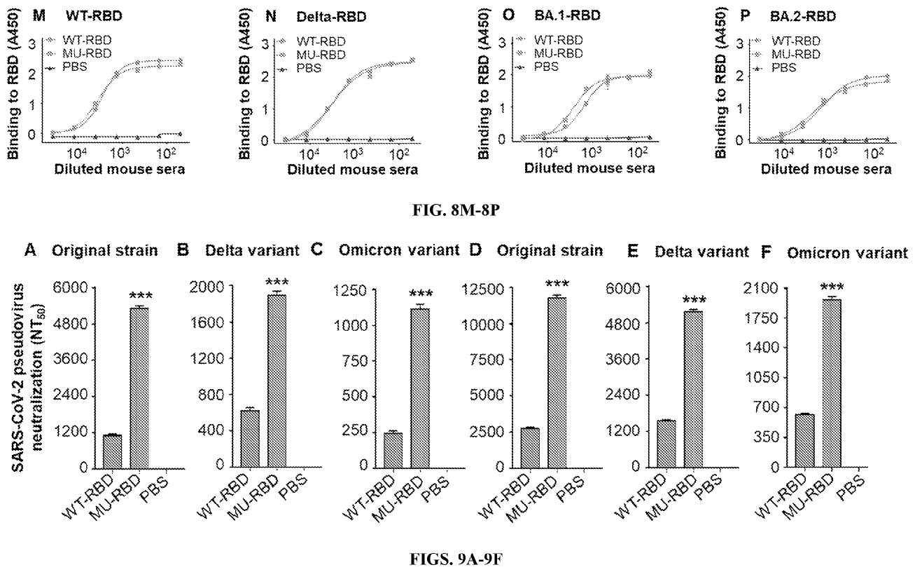

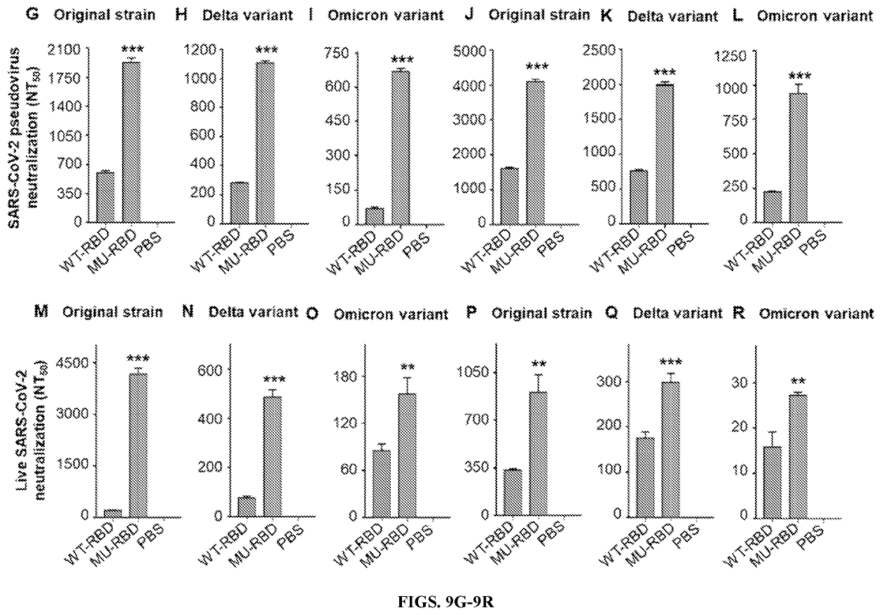

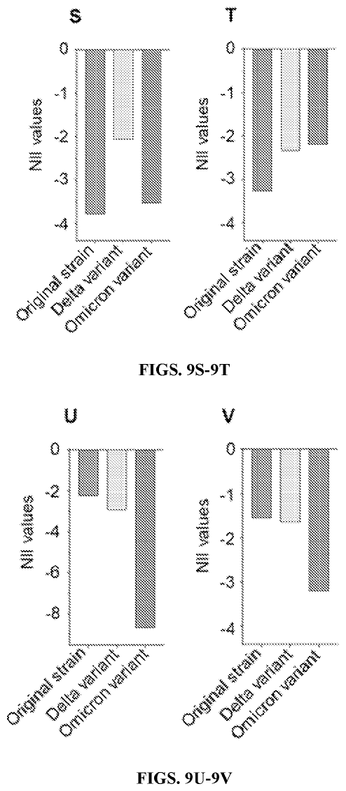

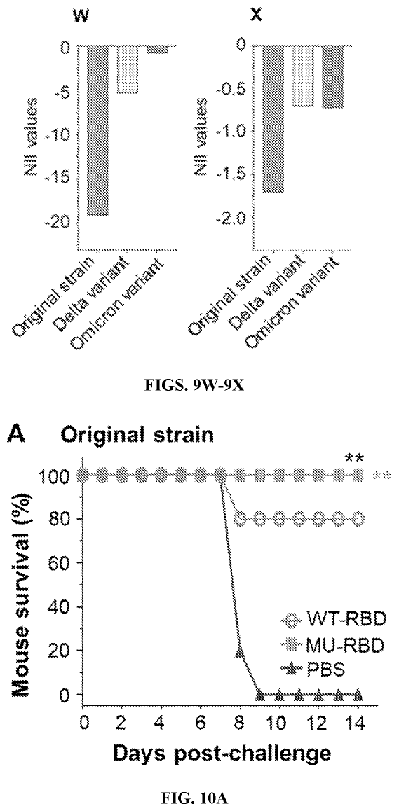

Compared with the prototypic RBD, the mutant RBD induced similar levels of IgG antibodies specific to the SARS-CoV-2 WT-RBD, Delta-RBD, Omicron BA.1-RBD, and Omicron BA.2-RBD proteins, respectively (FIGS. 8A-8P), but significantly higher-titer neutralizing antibodies against pseudotyped (FIGS. 9A-9L) and authentic (FIGS. 9M-9R) viruses of SARS-CoV-2 original strain, Delta, and Omicron variants (FIGS. 9A-9R) in both C57BL/6 and hACE2-Tg mice. Particularly, the third dose of the mutant RBD vaccine further improved neutralizing antibody titers compared to the second dose of immunization (FIG. 9A-9L). These results show that masking the non-neutralizing epitope Asn519 maintained the immunogenicity of RBD in the induction of specific IgG antibodies but increased the ability of the RBD to induce potent antiviral neutralizing antibodies against SARS-CoV-2 Delta and Omicron variants, in addition to original virus strain. Similarly, the negative NIIs of the mutant RBD against the original SARS-CoV-2 strain, Delta and Omicron variants confirm that the identified epitope makes a negative contribution to the overall neutralizing immunogenicity of the RBD (FIGS. 9S-9X). Notably, the neutralizing activity induced by the glycosylated mutant RBD were less effective against the Delta and Omicron variants than against original SARS-CoV-2 (FIGS. 9A-9R), and the numbers of IgG antibodies binding to the Omicron BA.1 and BA.2 RBD proteins were also lower than the numbers binding to the WT-RBD and Delta-RBD proteins (FIG. 8).

The above data indicate that glycosylated mutant RBD vaccine elicited antibodies that were able to effectively neutralize pseudotyped and authentic SARS-CoV-2 Delta and Omicron variants with multiple mutations in their S protein or RBD, albeit the neutralizing titers were relatively lower than those against the original virus strain.

Glycosylated mutant SARS-CoV-2 RBD showed greater efficacy than prototypic RBD in protecting mice against infection of SARS2-CoV-2 original strain and Delta variant. To investigate the ability of glycosylated mutant RBD to induce protective immunity against SARS-CoV-2 infection, immunized hACE2-Tg mice were challenged with lethal doses of the original strain or Delta variant of SARS-CoV-2 25-50 days after the last immunization, and survival and weights were monitored for 14 days. Immunized C57BL/6 mice were also challenged with a mouse-adapted SARS-CoV-2 variant (SARS2-N501Y_{MA30}), and viral titers were detected in the lung on day 2 after virus challenge (FIG. 6).

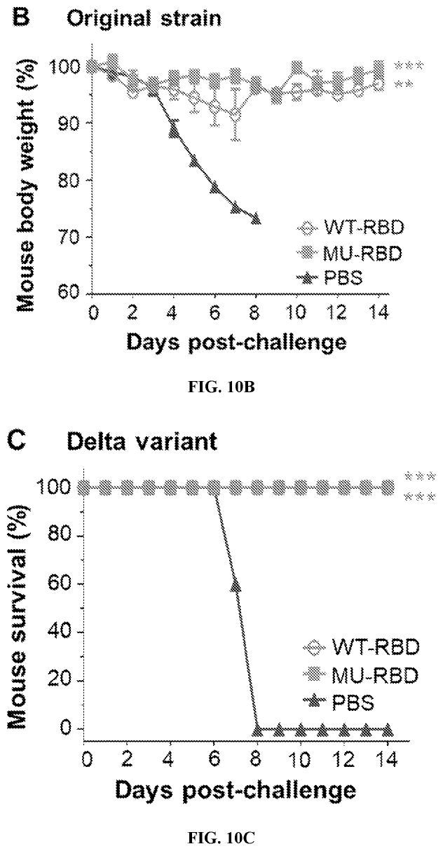

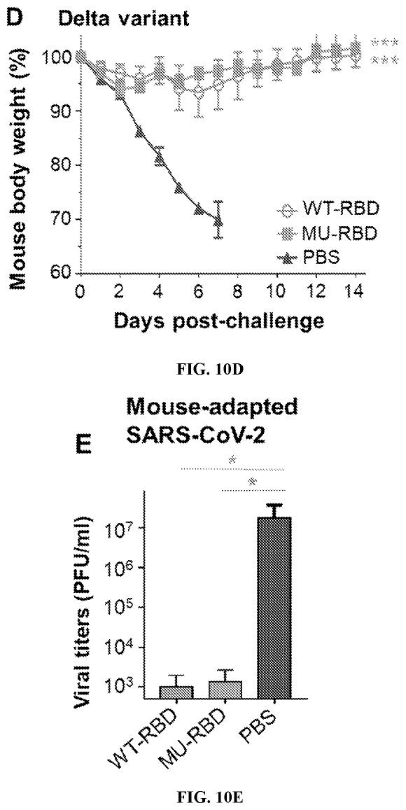

All of the hACE2-Tg mice immunized with the mutant RBD protein survived challenge with original SARS-CoV-2 infection and showed no weight loss, whereas only 80% of mice immunized with the prototypic RBD protein survived after this SARS-CoV-2 challenge with increased weight loss (FIGS. 10A-10B). Although all of the hACE2-Tg mice immunized with the mutant or prototypic RBD protein survived challenge with Delta SARS-CoV-2 variant, the mice immunized with the prototypic RBD protein had more weight loss than those immunized with the mutant RBD (FIGS. 10C-10D). In contrast, control hACE2-Tg mice receiving PBS and related adjuvants all lost weight after SARS-CoV-2 original or Delta variant challenge, and all died by 8 or 9 days after challenge (FIGS. 10A-10D). The immunized C57BL/6 mice potently inhibited replication of the mouse-adapted SARS2 (N501Y_{MA30}), with significantly lower viral titers in the lung than the PBS control mice after challenge (FIG. 10E). The above findings indicate that glycosylation of SARS-CoV-2 RBD of a single identified epitope enhanced neutralizing immunogenicity and protection of mice against SARS-CoV-2, leading to complete protection against infection of original virus strain and Delta variant without obvious weight loss.

### Discussion

Subunit vaccines are generally safe but have relatively low immunogenicity. The present example describes the design of a novel subunit vaccine with improved neutralizing immunogenicity and protection. Alignment of the SARS-CoV-2 and SARS-CoV RBD sequences indicates that SARS-CoV carries an asparagine (N) in its RBD corresponding to residue 519 (histidine, H) of SARS-CoV-2 RBD. In addition, this residue is located on a protruding loop in the core region of SARS-CoV-2 RBD (FIG. 5A), a potential immunodominant non-neutralizing epitope. Therefore, a glycan probe was placed onto residue 519 of SARS-CoV-2 RBD to form a N-linked glycan probe (N-X-T, where X is any amino acid other than proline) with residue 521, based on which a mutant RBD subunit vaccine was designed. This mutant RBD protein had the same receptor-binding affinity and antibody-binding activity as the prototypic RBD, presenting similar immunogenicity in the induction of effective IgG antibodies. Similar to the glycan-shielded subunit vaccines against MERS-CoV and Zika virus, the mutant RBD induced significantly higher titers of neutralizing antibodies than the prototypic RBD. The hACE2-Tg mice expressing SARS-CoV-2 receptor human ACE2, thus they are an effective lethal animal model for the evaluation of COVID-19 vaccines and therapeutic agents. Here it was found that the mutant RBD demonstrated improved efficacy in protecting immunized hACE2-Tg mice against lethal challenge with the prototype SARS-CoV-2 strain and Delta variant. These findings also confirm that the identified non-neutralizing epitope made a negative contribution to the overall neutralizing immunogenicity of RBD subunit vaccine, enhancing neutralizing activity and protective efficacy.

Several SARS-CoV-2 VOCs have been identified, all of which have mutations in the viral S protein, including the RBD. The Alpha, Beta, Gamma, and Delta VOCs induce more severe infections than the prototype virus strain, and the Delta variant may lead to more serious infection than other variants. In contrast, the Omicron variant appears to have increased transmissibility but cause attenuated infection and disease. Although at least three COVID-19 vaccines have been approved or authorized to prevent SARS-CoV-2 infection in humans, the emergence of these VOCs, particularly the Omicron variant, has limited current prevention strategies based on the prototype virus strain. Therefore, developing effective vaccines with broad-spectrum activity against different variants will be critical in preventing the spread of SARS-CoV-2 infection and ending the COVID-19 pandemic.

Interestingly, the glycosylated RBD described in this example was able to elicit neutralizing antibodies against pseudotyped and authentic prototype strain of SARS-CoV-2, as well as all mutant VOC strains tested, including Alpha, Beta, Gamma, Delta, and Omicron. Moreover, the neutralizing antibody titers against these strains were significantly higher than those induced by the prototypic RBD. These results show that the mutant RBD subunit had the ability to induce broadly neutralizing antibodies against multiple SARS-CoV-2 variants, including Omicron.

Overall, these results are consistent with other findings, which have demonstrated that antibodies induced by immunization with two doses of current COVID-19 vaccines had reduced neutralizing activity against Omicron and other variants than against prototype SARS-CoV-2. Moreover, although neutralizing antibodies against the Omicron variant could be induced by three doses of the Moderna or Pfizer mRNA vaccines, their titers were relatively low. Therefore, effective vaccines specifically targeting variant S proteins or their RBDs, or universal vaccines targeting the conserved epitopes of SARS-CoV-2 S protein, are demanded for further development. Improvements in the effectiveness of COVID-19 vaccines are urgently needed to prevent infection or at least disease caused by SARS-CoV-2 Omicron and other variants.

### Materials and Methods

Construction and purification of recombinant proteins. The RBD sequence of prototypic SARS-CoV-2 was amplified by PCR using a plasmid encoding S protein of SARS-CoV-2 (GenBank accession number QHR63250.2). The RBD sequence of SARS-CoV-2 Omicron BA.1 variant was amplified by PCR using a plasmid encoding RBD of SARS-CoV-2 Omicron BA.1 variant (GISAID accession number EPI_ISL_6795835). The RBD sequence of SARS-CoV-2 Omicron BA.2 variant (GISAID accession number EPI_ISL_9401700) was amplified using ClonExpress MultiS One Step Cloning Kit (Cellagen Technology LLC) based on the above prototypic SARS-CoV-2 RBD sequence. The amplified PCR fragments were fused with a C-terminal Fc fragment of human IgG. The mutant RBD and Delta RBD was constructed using multi-site-directed mutagenesis kit (Agilent Technologies) based on the prototypic SARS-CoV-2 RBD sequence. The recombinant plasmids were transfected into HEK293T/293F cells and the respective protein was purified from the culture supernatants using nProtein A Sepharose 4 Fast Flow (GE Healthcare).

ELISA. The binding between each SARS-CoV-2 RBD protein and human ACE2 (hACE2) protein was detected by ELISA (36). Briefly, the ELISA plates were coated with respective RBD protein (2 μg/ml) at 4° C. overnight, and blocked with 2% fat-free milk in PBS containing 0.05% Tween-20 (PBST) at 37° C. for 2 h. The plates were then incubated with hACE2 protein (20 or 5 μg/ml, R&D systems) at 37° C. for 2 h. After washing 3 times using PBST, the binding was detected by goat anti-hACE2 IgG antibody (0.2 μg/ml, R&D systems), followed by HRP-conjugated rabbit anti-goat IgG antibody (1:5,000, R&D systems). The plates were further washed and incubated with TMB (3,3′,5,5′-Tetramethylbenzidine) substrate (Sigma), and the reaction was stopped by 1 N H₂SO₄. The absorbance at 450 nm (A450) was measured using Cytation 7 Microplate Multi-Mode Reader (BioTek Instruments).

The binding between SARS-CoV-2 RBD proteins and RBD-specific serum antibodies or neutralizing antibodies (nAbs) was detected by ELISA. Briefly, the ELISA plates were coated with each RBD protein (1 μg/ml) at 4° C. overnight. After the aforementioned blockage and washing steps, the plates were incubated with serially diluted mouse or human sera, or nAbs (e.g., CV30, EY6A, and Ty1) (Absolute Antibody) at 37° C. for 2 h, followed by incubation with HRP-conjugated anti-mouse IgG antibody (1:5,000, Abcam; for mouse sera), anti-human IgG-Fab antibody (1:4,000, Abcam; for CV30, EY6A, and human sera), or anti-His antibody (1:4,000; for Ty1) at 37° C. for 1 h. Alternatively, the ELISA plates were coated with Nanosota-1C-Fc as described above, followed by sequential addition of each RBD protein, mouse anti-SARS-CoV-2-S polyclonal antibody, and HRP-conjugated anti-mouse antibody (1:5,000, Abcam). The other procedures were performed as described above.

Construction of recombinant plasmids. Recombinant plasmids expressing S protein of SARS-CoV-2 original strain (GenBank accession number QHR63250.2) or Alpha (B.1.1.7) variant strain (GISAID accession number EPI_ISL_718813) were constructed by inserting the respective DNA sequence into pcDNA3.1/V5-His-TOPO vector (Thermo Fisher Scientific). Omicron BA.1 variant (B.1.1.529) (GISAID accession number EPI_ISL_6795835) and recombinant plasmids expressing S protein of other variants containing single or multiple amino acid mutations were constructed using multi-site-directed mutagenesis kit (Agilent Technologies) and confirmed by sequencing analysis. The constructed plasmids were used to generate pseudoviruses as described below.

Generation of SARS-CoV-2 pseudoviruses and neutralization assay. These were performed as previously described with some modifications. Briefly, SARS-CoV-2 pseudoviruses were produced by co-transfection of 293T cells with pLenti-CMV-Luciferase, PS-PAX2, and each of the plasmids encoding SARS-CoV-2 original or mutant S protein using polyetherimide (PEI) transfection method. The pseudovirus-containing supernatants were harvested at 72 h post-transfection and used for neutralization assays. For pseudovirus neutralization, SARS-CoV-2 pseudoviruses expressing original or mutant S protein were incubated with serially diluted sera at 37° C. for 2 h, and then added to hACE2/293T cells in 96-well plates, followed by the addition of fresh medium 24 h later. 72 h later, the cells were lysed using cell lysis buffer (Promega), and incubated with luciferase substrate (Promega). The relative luciferase activity was measured using the Cytation 7 Microplate Multi-Mode Reader described above (BioTek Instruments). SARS-CoV-2 pseudovirus neutralization was calculated and expressed as 50% neutralizing antibody titer (NT₅₀).

Plaque reduction neutralization assay. Sera collected from below immunized mice were measured for neutralizing activity against prototype SARS-CoV-2 human strain (2019n-CoV/USA-WA1/2009), as well as authentic Delta (B.1.617.2) and Omicron (B.1.1.529) variants, using plaque reduction neutralization assay as previously described with some modifications. Briefly, sera which were serially diluted in Dulbecco's Modified Eagle Medium (DMEM) cell culture media were mixed with each virus (40-80 plaque-forming unit (PFU)/well) at 37° C. for 1 h. The serum-virus mixture was incubated with Vero E6 (for prototype strain and Delta variant) or Vero E6 in the presence of ACE2 and TMPRSS2 (for Omicron variant) cells at 37° C. for 45 min. After removing the inoculum, the cells were overlaid with 0.6% agarose, and cultured for three days. The overlays were then removed, and plaques were visualized by staining with 0.1% crystal violet. The neutralizing antibody titer was calculated as NT₅₀ (i.e., the highest serum dilution being able to reduce the number of virus plaques by 50%).

C57BL/6 and hACE2-transgenic (Tg) mice were used in this example. The animal welfare and experimental procedures were approved by the Committee on the Ethics of Animal Experiments. All mouse-related experiments were performed in strict accordance with the Guidelines for the Care and Use of Laboratory Animals of National Institutes of Health.

Mouse immunization and challenge studies. The following three immunization and challenge studies were performed as previously described with some modifications. First, four-month-old hACE2-Tg mice (mixed, 1-5 male or female) were intramuscularly (I.M.) immunized with SARS-CoV-2 prototypic-RBD protein, mutant RBD protein (10 μg/mouse), or PBS control in the presence of aluminum (500 μg/mouse) and monophosphoryl lipid A (MPL, 10 μg/mouse) adjuvants (InvivoGen). The immunized mice were boosted twice with the same immunogen and adjuvants at three weeks, and sera were collected at 10 days after the 2^{nd} and 3^{rd} immunizations to detect specific IgG antibodies or neutralizing antibodies (as described above). At 25 days after the last immunization, mice were transferred to Animal Biosafety Level 3 facility, and intranasally (I.N.) challenged with a prototype SARS-CoV-2 human strain (2019n-CoV/USA-WA1/2009, 5,000 PFU/mouse, 50 μl/mouse), and observed for survival and body weight changes for 14 days after the challenge. Second, two to three-month-old hACE2-Tg mice (mixed, 1-5 male or female) were immunized with each protein or control, collected for sera as described above, challenged with SARS-CoV-2 Delta (B.1.617.2) variant (10,000 PFU/mouse, 50 μl/mouse) at 50 days after the last immunization, and then observed for survival and body weight changes for 14 days after the challenge. Third, two to four-month-old female C57BL/6 mice were immunized with each protein or control, collected for sera as described above, and challenged with a virulent mouse-adapted strain of SARS-CoV-2 (SARS2-N501Y_{MA30}, 5,000 PFU/mouse, 50 μl/mouse) at 50 days after the last immunization. Mouse lungs were collected on day 2 after virus challenge for detection of viral titers as described below. The immunization and challenge schedules were summarized in FIG. 6.

Detection of viral titers. The lung tissues from challenged mice were homogenized and centrifuged for collection of supernatants. The supernatants were then serially diluted in DMEM cell culture medium and incubated with Vero E6 cells at 37° C. for 1 h. This was followed by the same procedures as plaque reduction neutralization assay described above. Viral titers were quantified as PFU/ml of lung tissue.

Statistical analysis. Statistical significances were analyzed using GraphPad Prism 9 statistical software. Neutralizing antibody or viral titers between the mutant RBD protein, prototypic RBD protein, and/or PBS control groups were calculated using two-tailed independent student t test. Statistical significance between the survival curves was calculated by Kruskal-Wallis test. Statistical significance between the weight curves was calculated by Ordinary one-way ANOVA. P<0.05 was considered as significant. *, **, and *** in the figures represent P<0.05, P<0.01, and P<0.001, respectively.

### Example 3. MRNA Vaccines Elicit Potent Neutralization Against Multiple SARS-CoV-2 Omicron Subvariants and Other Variants of Concern

SARS-CoV-2 variants of concern (VOCs) have shown resistance to vaccines targeting the original virus strain. An mRNA vaccine encoding the spike protein of Omicron BA1 (BA1-S-mRNA) was designed, and its neutralizing activity, with or without the original receptor-binding domain (RBD)-mRNA, was tested against SARS-CoV-2 VOCs. First-dose of BA1-S-mRNA followed by two-boosts of RBD-mRNA elicited potent neutralizing antibodies (nAbs) against pseudotyped and authentic original SARS-CoV-2; pseudotyped Omicron BA1, BA2, BA2.12.1 and BA5 subvariants, and Alpha, Beta, Gamma, and Delta VOCs; authentic Omicron BA1 subvariant and Delta VOC. By contrast, other vaccination strategies, including RBD-mRNA first-dose plus BA1-S-mRNA two-boosts, RBD-mRNA or BA1-S-mRNA three-doses, or their combinations, failed to elicit high nAb titers against all of these viruses. Overall, this vaccination strategy was effective for inducing broadly and potent nAbs against multiple SARS-CoV-2 VOCs, particularly Omicron BA5, and may guide the rational design of next-generation mRNA vaccines with greater efficacy against future variants.

Coronavirus Disease 2019 (COVID-19), which first emerged in 2019, has resulted in a worldwide pandemic with devastating economic losses and threats to public health. COVID-19 is caused by severe acute respiratory syndrome coronavirus-2 (SARS-CoV-2), one of the three highly pathogenic coronaviruses (CoVs) in the beta-CoV genus of the Coronaviridae family. As of Jan. 27, 2023, SARS-CoV-2 has infected more than 753 million individuals worldwide and caused more than 6.8 million deaths.

SARS-CoV-2 infection of host cells is initiated when receptor-binding domain (RBD) of the S1 subunit of the viral surface spike (S) protein binds to its receptor, angiotensin converting enzyme 2 (ACE2), on host cells. The S2 subunit of the viral S protein subsequently mediates fusion between the virus and cell membranes. Native S protein presents as a trimeric structure, consisting of three receptor-binding domain (RBD) molecules. Therefore, the S protein and its RBD fragments are key targets for the development of anti-SARS-CoV-2 vaccines and therapeutic antibodies.

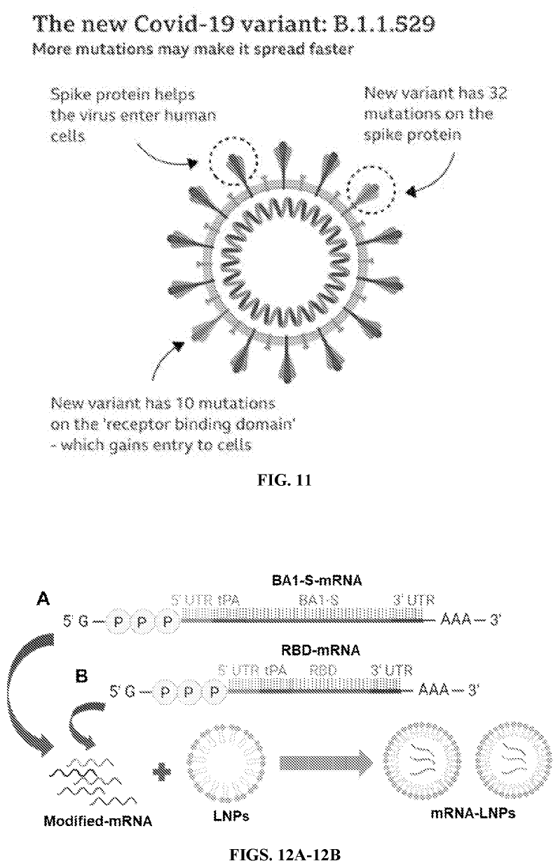

SARS-CoV-2 mutates rapidly and frequently, with multiple mutations being detected in its S protein and other proteins (FIG. 11). These mutations have resulted in different variants of concern (VOCs), such as the Alpha (B.1.1.7), Beta (B.1.351), Gamma (P.1), Delta (B.1.617.2), and Omicron (B.1.1.529) variants, with the Omicron variant subclassified as several subvariants, including BA1, BA2, BA3, BA4, and BA5. Omicron BA5 is the recent predominant subvariant, highly resistant to COVID-19 vaccines and therapeutic antibodies targeting the original SARS-CoV-2 S protein. Thus, the development of effective vaccines with high potency against BA5 and other VOCs with pandemic potential is important to prevent the global spread of SARS-CoV-2.

Previously, an mRNA vaccine encoding the original RBD fragment of SARS-CoV-2 (RBD-mRNA) was designed. This vaccine induced the production of highly potent neutralizing antibodies (nAbs) against the original strain of SARS-CoV-2, as well as protecting against a mouse-adapted SARS-CoV-2 infection. However, its neutralizing activity against Delta (B.1.617.2) and Omicron (B.1.1.529) VOCs was significantly lower than its activity against the original strain. The present example describes the design of a new mRNA vaccine encoding the S protein of SARS-CoV-2 Omicron BA1 containing HexaPro sequences and a foldon trimeric structure (BA1-S-mRNA). Mice were immunized with this new mRNA vaccine, either sequentially or in combination with RBD-mRNA, and the vaccine-induced immunogenicity and neutralizing activity against several Omicron subvariants and various other VOCs were evaluated.

### Results

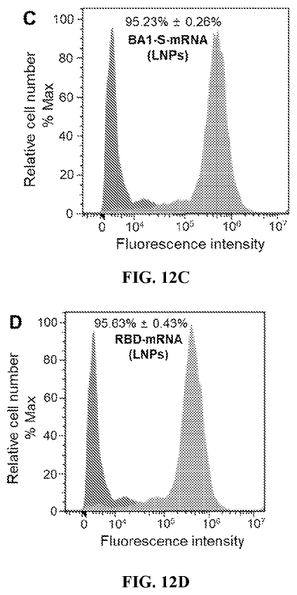

Characterization of SARS-CoV-2 mRNA vaccines. BA1-S-mRNA was designed to encode a tissue plasminogen activator (tPA) signal peptide, the S protein of Omicron BA1, foldon, and His6 tag sequences; this mRNA also contained 5′- and 3′-untranslated regions (UTRs) (FIG. 12A) with the control consisting of mRNA encoding the RBD of the original strain of SARS-CoV-2 (RBD-mRNA) and a His6 tag (FIG. 12B). Each synthesized mRNA had a 5′-Cap 1 structure and a 3′-poly(A) tail, which was encapsulated with lipid nanoparticles (LNPs) for delivery (FIGS. 12A-12B). Flow cytometry analysis showed that cells incubated with LNP-encapsulated BA1-S-mRNA or RBD-mRNA were strongly fluorescent, indicating the expression of specific proteins, whereas control cells were not (FIGS. 12C-12D).

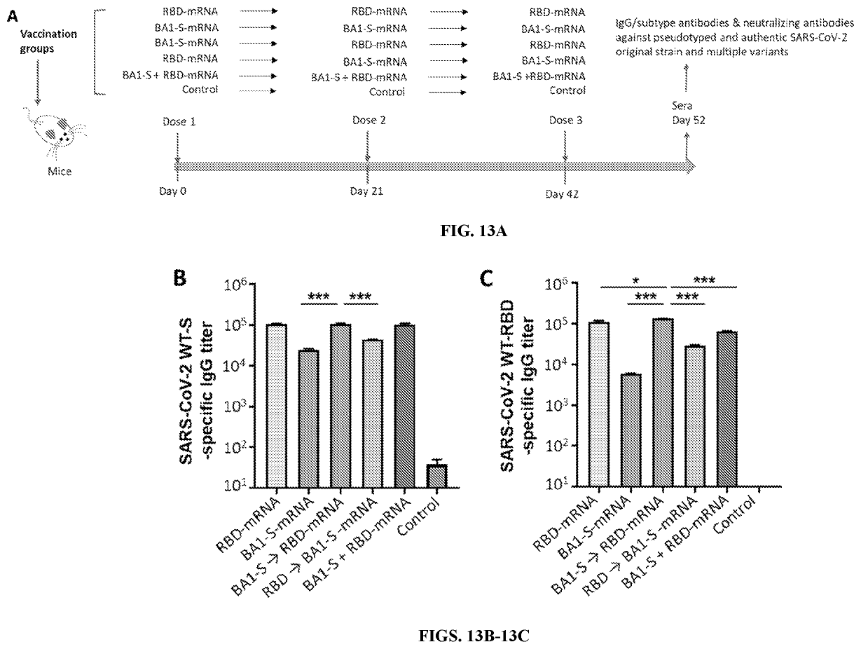

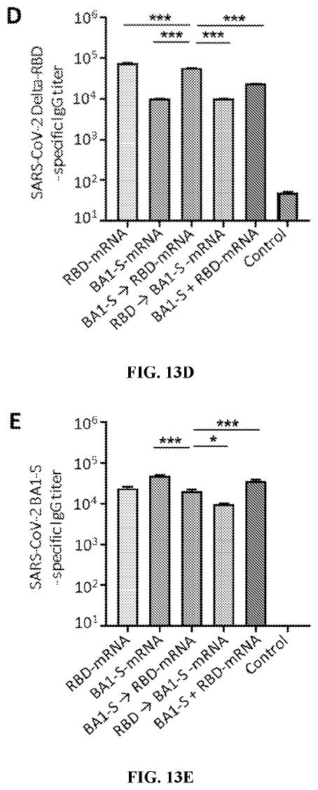

Antibody responses induced by SARS-CoV-2 BA1-S-mRNA followed by RBD-mRNA sequential immunization. To determine the immunogenicity of BA1-S-mRNA, with or without sequential immunization with RBD-mRNA, mice were immunized with 1) three doses of RBD-mRNA alone, 2) three doses of BA1-S-mRNA alone, 3) one dose of BA1-S-mRNA followed by two doses of RBD-mRNA, 4) one dose of RBD-mRNA followed by two doses of BA1-S-mRNA, 5) three doses of a 1:1 combination of BA1-S-mRNA and RBD-mRNA, or 6) three doses of PBS (control) (FIG. 13A). Doses were administered at 3-week intervals, and sera collected 10 days after the third immunization were pooled for each group and assessed for IgG and subtype antibodies specific to the S or RBD protein of the original strain of SARS-CoV-2, as well as to the Delta (B.1.617.2) and Omicron (B.1.1.529) variants (FIG. 13A).

Evaluation of vaccine-induced IgG antibodies demonstrated that three doses of RBD-mRNA alone induced higher titers of IgG antibodies specific to the wild-type S (WT-S), wild-type RBD (WT-RBD), and Delta-RBD proteins than to the S protein of Omicron-BA1 subvariant (BA1-S), whereas three doses of BA1-S-mRNA alone elicited higher IgG antibodies specific to the BA1-S protein than to the other proteins tested (FIGS. 13B-13E). A single dose of BA1-S-mRNA followed by two doses of RBD-mRNA elicited potent antibodies specific to the WT-S, WT-RBD, and Delta-RBD proteins (FIGS. 13B-13D), as well as favorable but relatively low-titer IgG antibodies specific to the BA1-S protein (FIG. 13E). By contrast, one dose of RBD-mRNA followed by two doses of BA1-S-mRNA induced significantly low-titer IgG antibodies than single dose of BA1-S-mRNA plus two doses of RBD-mRNA against any of the proteins tested (FIGS. 13B-13E). Three doses of a 1:1 combination of BA1-S-mRNA and RBD-mRNA induced the production of higher-titer IgG antibodies targeting the WT-S protein than those against the other three proteins (FIGS. 13B-13E). The control, PBS, did not induce antibodies specific to any of these proteins (FIGS. 13B-13E).

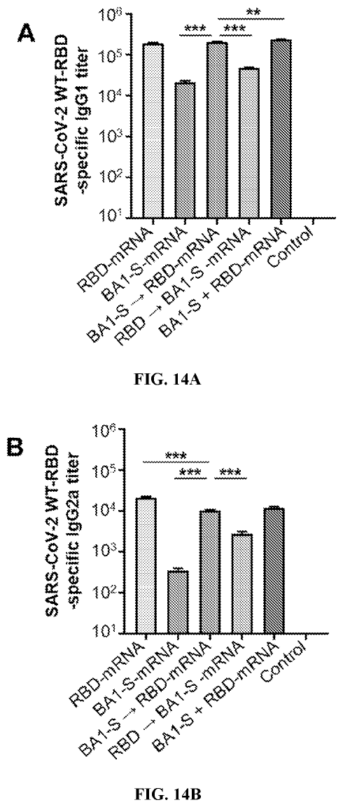

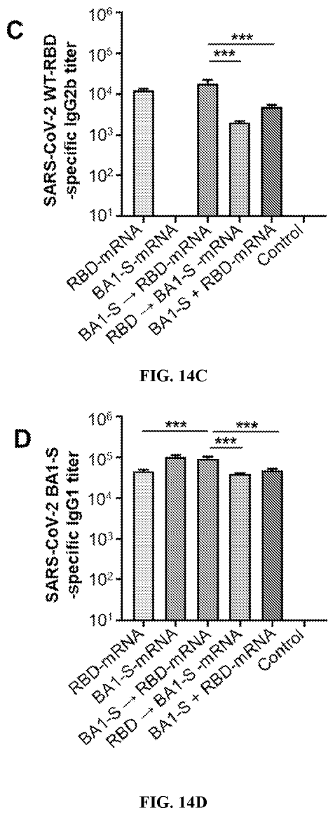

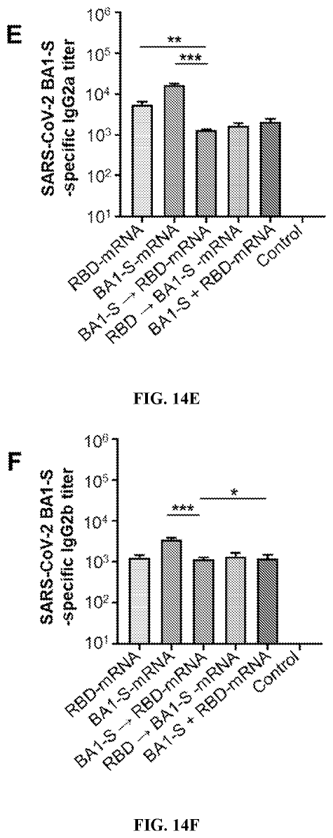

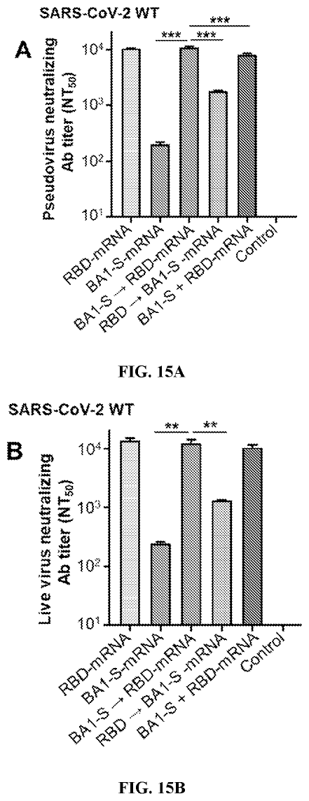

Evaluation of vaccine-induced IgG subtype antibodies revealed that three doses of RBD-mRNA alone elicited more potent IgG1 antibodies than IgG2a and IgG2b antibodies specific to the WT-RBD protein, rather than to the BA1-S protein, whereas three doses of BA1-S-mRNA alone induced potently higher IgG1 antibodies than IgG 2a and IgG2b antibodies specific to the BA1-S protein, rather than to the WT-RBD protein (FIGS. 14A-14F). By contrast, one dose of BA1-S-mRNA plus two doses of RBD-mRNA elicited higher-titer IgG1 antibodies than IgG2a and IgG2b antibodies with similar potency against both WT-RBD and BA1-S proteins (FIGS. 14A-14F). Notably, one dose of RBD-mRNA plus two doses of BA1-S-mRNA induced significantly lower IgG1, IgG2a, or IgG2b antibodies than one dose of BA1-S-mRNA plus two doses of RBD-mRNA specific to the WT-RBD or BA1-S protein (FIGS. 14A-14D). In addition, IgG1, IgG2a, and IgG2b antibodies induced by combinatorial BA1-S-mRNA and RBD-mRNA were either similar or significantly lower than those induced by one dose of BA1-S-mRNA plus two doses of RBD-mRNA specific to the WT-RBD and BA1-S proteins (FIGS. 14B-14D). No specific IgG subtype antibodies were elicited in the PBS control mice (FIGS. 14A-14F).

Taken together, the above data indicate that BA1-S-mRNA priming followed by two doses of RBD-mRNA, was capable of inducing effective antibodies against the original strain of SARS-CoV-2, as well as the Delta and Omicron variants, whereas RBD-mRNA priming followed by two doses of BA1-S-mRNA and BA1-S-mRNA alone were not.

SARS-CoV-2 BA1-S-mRNA followed by RBD-mRNA sequential immunization enhanced neutralizing activity against multiple Omicron subvariants. To evaluate the ability of BA1-S-mRNA, with or without sequential immunization with RBD-mRNA, to induce neutralizing antibodies against the Omicron variant of SARS-CoV-2, sera collected 10 days after the third immunization (FIG. 13A) were pooled for each group and tested against both pseudotyped and authentic Omicron (B.1.1.529) subvariants, including BA1, BA2, BA2.12.1, and BA5, with the control consisting of assays of neutralizing antibodies against the original strain of SARS-CoV-2.

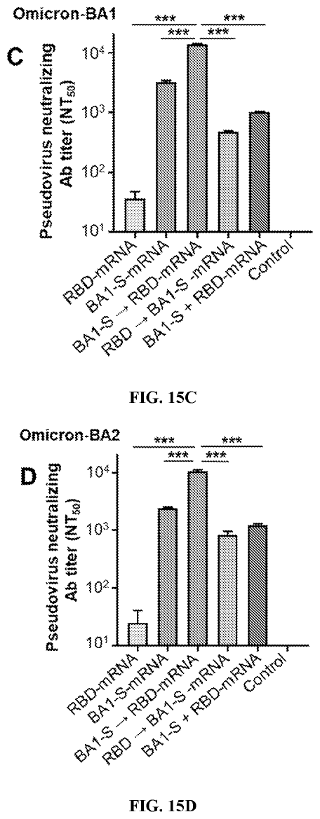

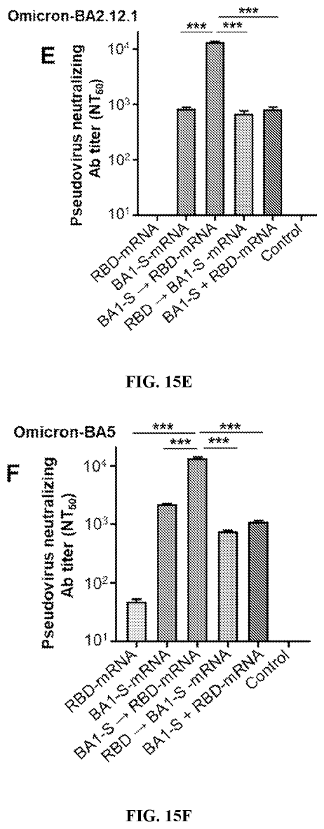

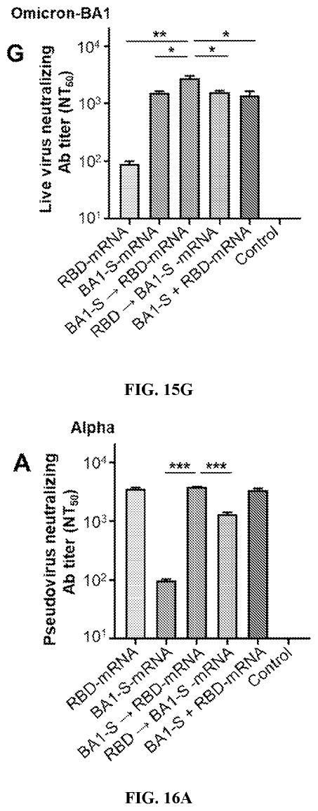

Three doses of RBD-mRNA alone induced high-titer neutralizing antibodies against the pseudotyped and authentic original (WT) strains of SARS-CoV-2 (FIGS. 15A-15B), but not against the Omicron subvariants (FIGS. 15C-15G). Nevertheless, three doses of BA1-S-mRNA alone induced relatively higher-titer neutralizing antibodies against the pseudotyped and authentic BA1 and other Omicron subvariants (FIGS. 15C-15G), but lower-titer neutralizing antibodies against the original strain of SARS-CoV-2 (FIGS. 15A-15B). By contrast, immunization with one dose of BA1-S-mRNA followed by two doses of RBD-mRNA induced potent neutralizing antibodies against the pseudotyped (FIG. 15A) and authentic (FIG. 15B) original strain of SARS-CoV-2, and these titers were similar to those induced by administration of three doses of RBD-mRNA alone (FIGS. 15A-15B). Notably, these neutralizing antibodies were significantly higher than those induced by other immunizations against all pseudotyped Omicron subvariants tested, including Omicron BA1, BA2, BA2.12.1, and BA5 (FIGS. 15C-15F), as well as authentic Omicron BA1 (FIG. 15G). In particular, the titers of neutralizing antibodies against Omicron BA5 were not significantly reduced compared with the titers against the original strain and other Omicron subvariants (FIGS. 15A-15F). Differently, one dose of RBD-mRNA followed by two doses of BA1-S-mRNA induced similarly low-level neutralizing antibodies against all SARS-CoV-2 strains tested (FIGS. 15A-15F). The combination of RBD-mRNA and BA1-S-mRNA induced potent neutralizing antibodies against the original SARS-CoV-2 (FIGS. 15A-15B), but relatively low-titer neutralizing antibodies against the Omicron subvariants tested (FIGS. 15C-15G). The negative control, PBS, did not elicit any SARS-CoV-2-specific neutralizing antibodies (FIG. 15).

These data indicate that sequential immunization of BA1-S-mRNA followed by two doses of RBD-mRNA was able to elicit potent and broadly neutralizing antibodies against both SARS-CoV-2 original strain and multiple Omicron subvariants, including BA5. By contrast, the other vaccinations tested, including RBD-mRNA alone, BA1-S-mRNA alone or their combination, or one dose of RBD-mRNA followed by two doses of BA1-S-mRNA, were unable to induce the production of higher-titer antibodies that neutralized against the original SARS-CoV-2 strain and multiple Omicron subvariants tested.

SARS-CoV-2 BA1-S-mRNA followed by RBD-mRNA sequential immunization induced potent and broadly neutralizing antibodies against other variants of concern. The ability of BA1-S-mRNA, with or without sequential immunization with RBD-mRNA, to elicit neutralizing antibodies against other VOCs of SARS-CoV-2 was also tested using pooled sera obtained 10 days after the third immunization (FIG. 13A).

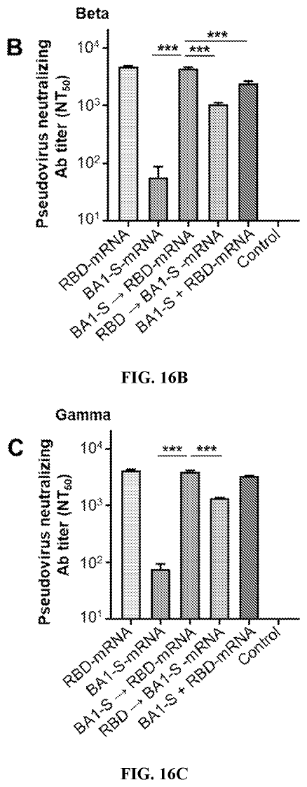

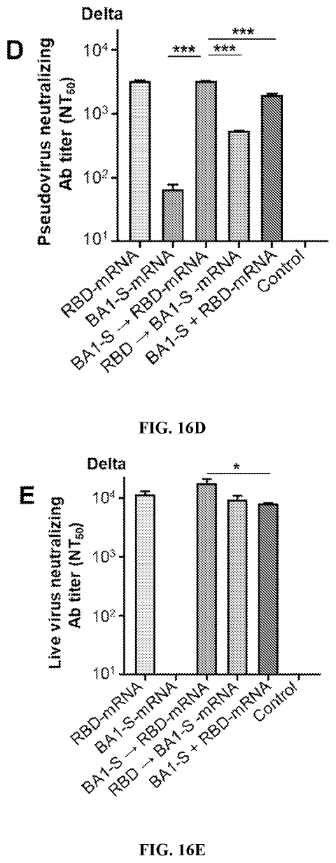

Three doses of RBD-mRNA alone induced similarly high neutralizing antibodies against pseudotyped Alpha (B.1.1.7), Beta (B.1.351), Gamma (P.1) and Delta (B.1.617.2) variants, and authentic Delta variant, whereas three doses of BA1-S-mRNA alone failed to elicit, or elicited very low-titer, neutralizing antibodies against these SARS-CoV-2 variants (FIGS. 16A-16E). Of note, administration of one dose of BA1-S-mRNA, followed by two boosts with RBD-mRNA, induced neutralizing antibodies against pseudotyped Alpha, Beta, Gamma, and Delta variants and authentic Delta variants that were as potent as, or higher than, those elicited by three doses of RBD-mRNA (FIGS. 16A-16E). By contrast, one dose of RBD-mRNA followed by two doses of BA1-S-mRNA, as well as three doses of 1:1 mixture of RBD-mRNA and BA1-S-mRNA, elicited lower, or significantly lower, neutralizing antibody titers against pseudotyped Alpha, Beta, Gamma, and Delta variants (FIGS. 16A-16D) and authentic Delta variant (FIG. 16E), than a single dose of BA1-S-mRNA followed by two doses of RBD-mRNA. Although neutralizing antibody titers in mice administered with one dose of RBD-mRNA followed by two doses of BA1-S-mRNA were higher than those in mice immunized with three doses of BA1-S-mRNA, they were lowest among the other vaccination groups (FIGS. 16A-16D). No SARS-CoV-2-specific neutralizing antibodies were induced in the control mice injected with PBS (FIGS. 16A-16E).

These findings indicate that sequential immunization with BA1-S-mRNA plus two doses of RBD-mRNA, rather than one dose of RBD-mRNA plus two doses of BA1-S-mRNA or three doses of BA1-S-mRNA, was able to induce highly and broadly neutralizing antibodies against other SARS-CoV-2 VOCs tested.

### Discussion

Variants of SARS-CoV-2 are much more frequent than variants of other human pathogenic coronaviruses, such as SARS-CoV and Middle East respiratory syndrome coronavirus (MERS-CoV). Among currently identified Omicron subvariants, the predominant subvariant is Omicron BA5, being present in more than 29% of reported COVID-19 cases in the U. S. Most COVID-19 vaccines developed to date targeted the original S protein or RBD of SARS-CoV-2, with limited neutralizing activity against newly emerging SARS-CoV-2 variants, particularly the Omicron subvariants. Efforts have therefore focused on the development of advanced COVID-19 vaccines with greater neutralizing capacity against dominant Omicron variants/subvariants or multiple SARS-CoV-2 VOCs.

Different approaches have been applied to enhance the neutralizing activity of COVID-19 vaccines against SARS-CoV-2 variants. Several strategies involving an initial dose of a vaccine targeting the original strain of SARS-CoV-2, following by boosts with the same vaccine or vaccines targeting different antigens in the original strain or a variant. For example, a third dose of the CureVac COVID-19 (CVnCoV) mRNA vaccine was found to elicit increased neutralizing antibodies against the original strain of SARS-CoV-2 and the Delta (B.1.617.2) variant, and the neutralizing antibody titers were similar in naïve and pre-exposed participants. In addition, a third dose of the Pfizer/BioNTech SARS-CoV-2 mRNA vaccine increased the titers of neutralizing antibodies against SARS-CoV-2 Omicron subvariants (BA1. BA1.1, or BA2) and/or Delta variant. Moreover, a third dose of the inactivated SARS-CoV-2 vaccine (CoronaVac) also increased the neutralizing antibody titers against the Delta and Omicron variants. Differently, three doses of the BNT162b2 mRNA vaccine, or first and second doses of CoronaVac vaccine followed by third dose of the BNT162b2 vaccine, enhanced the titers of neutralizing antibodies against the Beta (B.1.351) and Delta (B.1.617.2) variants, but reduced titers against the Omicron variant.

The present example describes a new approach, consisting of priming with the BA1-S-mRNA vaccine, which encodes the S protein of the SARS-CoV-2 Omicron BA1 subvariant, followed by two doses of RBD-mRNA vaccine encoding the RBD of the original strain of SARS-CoV-2. Strikingly, this approach resulted in significant enhancement of neutralizing antibodies against all of the SARS-CoV-2 Omicron subvariants tested, including BA1, BA2, BA2.12.1, and BA5, as well as maintaining the potency of RBD-mRNA to neutralize the original strain of SARS-CoV-2 and several other VOCs, including Alpha (B.1.1.7), Beta (B.1.351), Gamma (P.1), and Delta (B.1.617.2). By contrast, a first dose of RBD-mRNA followed by two boosts of BA1-S-mRNA was unable to induce effective neutralizing antibodies against these SARS-CoV variants. Moreover, RBD-mRNA alone elicited high-titer neutralizing antibodies against the original strain of SARS-CoV-2, but not against Omicron subvariants, whereas BA1-S-mRNA alone elicited neutralizing antibodies against Omicron subvariants but not against the original strain of SARS-CoV-2. Multiple mutations have been identified in the RBD region of this S protein, with these mutations likely affecting the immunogenicity and neutralizing activity of the mutant RBD-containing S protein.

The present example compared various regimens of SARS-CoV-2 mRNA vaccines encoding the original RBD of SARS-CoV-2 and S protein of the Omicron BA1 subvariant, identifying an effective immunization strategy for the induction of broadly and potent neutralizing antibodies against multiple SARS-CoV-2 variants, particularly the currently predominant Omicron BA5 subvariant. This strategy prevents infection by multiple current SARS-CoV-2 variants, as well as guiding the rational design and testing of next-generation mRNA vaccines with improved efficiency and efficacy against future variants of this virus.

### Methods

Cell lines. HEK293T, hACE2/293T (e.g., 293T cells expressing human ACE2 receptor), and Vero E6 cells were cultured at 37° C. and 5% CO₂ in a humidified incubator. The culture medium (Dulbecco's Modified Eagle Medium: DMEM) contained 10% fetal bovine serum (FBS) (R&D Systems) and 1% Penicillin-Streptomycin (Thermo Fisher Scientific).

Viruses. The following SARS-CoV-2 pseudoviruses were used, which expressed the S protein or RBD of the original strain (GenBank accession number QHR63250.2), Alpha (B.1.1.7) variant (GISAID accession number EPI_ISL_718813), Omicron (B.1.1.529) subvariants BA1 (GISAID accession number EPI_ISL_6795835), BA2 (GISAID accession number EPI_ISL_12030355), BA2.12.1 (GISAID accession number EPI_ISL_12061569), and BA5 (GISAID accession number EPI_ISL_12043290). In addition, Beta (B.1.351), Gamma (P.1), and Delta (B.1.617.2) variants were constructed by mutating the RBD residues of the above original strain of SARS-CoV-2. Authentic SARS-CoV-2 original strain (USA-WA1/2020) (GenBank accession number MN985325), Delta (hCoV-19/USA/MD-HP05647/2021) (GISAID accession number EPI_ISL_2331496) and Omicron (B.1.1.529) (PP3P1hCoV19/EHC_C19_2811C) variants were used in this example.

Mice. Female BALB/c mice at 6-8 weeks of age were purchased from the Jackson Laboratory, and randomly assigned to experimental groups. The animal protocols were approved by the Institutional Animal Care and Use Committees (IACUC). All mouse-related experiments were performed in strict accordance with the regulatory standards and guidelines for the Care and Use of Laboratory Animals of the National Institutes of Health (NIH), as well as approved protocols.

Design and synthesis of SARS-CoV-2 mRNAs. The nucleoside-modified mRNAs encoding the RBD or S protein of SARS-CoV-2 were constructed as follows. Specifically, the DNA sequence of SARS-CoV-2 Omicron S containing a N-terminal tPA signal peptide, a C-terminal foldon trimeric sequence, and His6 tag (BA1-S-mRNA) was amplified by PCR using a plasmid encoding codon-optimized S protein with HexaPro sequences (a mutated furin cleavage site and six proline substitutions) of SARS-CoV-2 Omicron variant (GISAID accession number EPI_ISL_6795835). The DNA sequence of SARS-CoV-2 RBD containing tPA signal peptide and a C-terminal His6 tag (RBD-mRNA) was constructed by PCR using a plasmid encoding codon-optimized S protein of SARS-CoV-2 original strain (GenBank accession number QHR63250.2). The purified PCR products were inserted into a pCAGGS-mCherry vector to construct recombinant plasmids, which also contained N-terminal T7 promotor, 5′ and 3′-UTRs (FIG. 12).

The nucleoside-modified mRNAs were synthesized as described below. Specifically, the above recombinant plasmids were linearized using BglII restriction enzyme and synthesized using MEGAscript T7 Transcription Kit according to the manufacturer's instructions (Thermo Fisher Scientific). Nucleosides CTP, ATP and GTP, as well as pseudo-UTP (Ψ) (APExBIO), were added during the mRNA synthesis to increase the stability of mRNAs and enhance the expression of target antigens. Following the manufacturer's instructions, the purified mRNAs were then capped with Cap 1 Capping System Kit (containing ScriptCap Capping Enzyme and 2′-O-Methyltransferase) (CELLSCRIPT) and tailed with Poly(A) Polymerase Tailing Kit (CELLSCRIPT), which generated a Cap 1 structure and poly(A) tail (150 bp).

Encapsulating mRNAs with lipid nanoparticles (LNPs). The synthesized mRNAs were encapsulated with LNPs (mRNA-LNPs) as described below. Specifically, each mRNA diluted in PNI Formulation Buffer (Precision Nanosystems) was encapsulated with lipid mixture (GenVoy-ILM) (Precision Nanosystems) (3:1 ratio) using NanoAssemblr Ignite Instrument according to the manufacturer's instructions (Precision Nanosystems). This was followed by filtration and concentration of mRNA-LNPs using Amicon Ultra-15 Centrifugal Filters (10 kDa). The endotoxin level of mRNA-LNPs was detected by LAL Endotoxin Assay Kit (GenScript) (<1 EU/ml), with the particle size around 80-110 nm.

Detection of protein expression. Flow cytometry was used to detect the expression of LNP-encapsulated mRNAs in 293T cells. Specifically, the cells were pre-plated into 24-well culture plates (2×10⁵/well) containing complete DMEM cell culture medium 24 h before experiments, incubated with each mRNA-LNP (1 μg/ml), and cultured at 37° C. in the presence of 5% CO₂. 48 h later, the cells were stained with mouse-anti-His-FITC antibody (Thermo Fisher Scientific), and analyzed by flow cytometry (CytoFLEX flow cytometer, Beckman Coulter Life Sciences) using FlowJo software (BD Biosciences).

ELISA. Enzyme-linked immunoassay (ELISA) was used to measure specific serum antibodies from immunized mice. Specifically, 96-well ELISA plates were precoated with each recombinant SARS-CoV-2 S or RBD protein (1 μg/ml) at 4° C. overnight, and blocked with blocking buffer (e.g., 2% fat-free milk dissolved in PBST (0.05% Tween-20 in PBS)) at 37° C. for 1 h. The plates were then incubated with serially diluted mouse sera at 37° C. for 1 h and washed with PBST for at least three times. This step was followed by further incubation of the plates with horseradish peroxidase (HRP)-conjugated anti-mouse IgG-Fab (1:5,000, Sigma), anti-mouse IgG1, anti-mouse IgG2a, and anti-mouse IgG2b (1:5,000, Invitrogen) antibodies, respectively, at 37° C. for 1 h, and washing for three times. After incubation of the plates with 3,3′,5,5′-Tetramethylbenzidine (TMB) substrate (Sigma), the reaction was stopped by addition of 1 N H₂SO₄. The absorbance at 450 nm was measured using Cytation 7 Microplate Multi-Mode Reader and Gen5 software (BioTek Instruments).

Pseudovirus generation and neutralization assay. SARS-CoV-2 pseudoviruses were generated as described below. Specifically, plasmid encoding S protein of the original strain or respective variant of SARS-CoV-2 was transfected into 293T cells in the presence of pLenti-CMV-luciferase plasmid and PS-PAX2 plasmid (Addgene) using the polyetherimide (PEI) (Sigma) transfection assay. 72 h after the transfection, pseudovirus-containing supernatants were collected, and used for pseudovirus neutralization assay. Each pseudovirus was incubated with serially diluted mouse sera at 37° C. for 1 h, which was added to hACE2/293T cells; 24 h later, fresh medium was added to the cells, and the cells were cultured at 37° C. in the presence of 5% CO₂, followed by lysis using cell lysis buffer (Promega) 48 later. The supernatant of lysed cells was incubated with luciferase substrate (Luciferase Assay System) (Promega), which was then measured for relative luciferase activity using Cytation 7 Microplate Multi-Mode Reader and Gen5 software (BioTek Instruments). Pseudovirus neutralizing activity was reported as 50% neutralizing antibody titer (NT₅₀).

Live virus neutralization assay. Sera from immunized mice were tested for neutralizing activity against authentic SARS-CoV-2 infection using plaque reduction neutralization assay as described below. Specifically, serially diluted sera were incubated with the SARS-CoV-2 original strain, Delta (B.1.617.2) or Omicron (B.1.1.529) variant (40-80 plaque-forming unit (PFU)/well) at 37° C. for 1 h. The virus-serum mixture was then added to Vero E6 (for original strain and Delta variant) or Vero E6 cells in the presence of ACE2 and TMPRSS2 (for Omicron variant) at 37° C. for 45 min. The inoculum was removed, followed by overlaying the cells with 0.6% agarose (Research Products International) and culturing them for three days. The plaques were stained with 0.1% crystal violet (Fisher Scientific). The neutralizing antibody titer was calculated as NT₅₀ (representing highest serum dilution that reduced the number of virus plaques by 50%).

Mouse immunization and related sample collection. Mice were immunized with respective mRNA-LNP as described below. Specifically, mice were intradermally (I. D.) immunized with the following groups of mRNA-LNPs (10 μg/mouse) or control: 1) RBD-mRNA (3 doses), 2) BA1-S-mRNA (3 doses), 3) BA1-S-mRNA (prime) and RBD-mRNA (2 boosts), 4) RBD-mRNA (prime) and BA1-S-mRNA (2 boosts), 5) combined RBD-mRNA and BA1-S-mRNA (3 doses), and 6) PBS control (FIG. 2). The immunized mice were boosted twice at a 3-week interval, and sera were collected 10 days after the last immunization to detect specific IgG and subtype antibodies, as well as neutralizing antibodies, using ELISA and pseudovirus or live virus neutralization assays as described above.

Quantitative and statistical analysis. GraphPad Prism 9 statistical software was used for analysis of the data generated in this study. The data are presented as mean±s.e.m. Statistical significance among different vaccination groups was calculated using Ordinary one-way ANOVA with Dunnett's multiple comparisons test. P<0.05 was considered significant. *, **, and *** indicate P<0.05, P<0.01, and P<0.001, respectively.

### Example 4. A Broadly Universal Vaccine Protects Against Multiple SARS-CoV-2 Variants and SARS-CoV

Rapid SARS-CoV-2 mutations lead to significant reduction of neutralization and protection of COVID-19 vaccines against multiple variants of concern (VOCs). In addition, SARS-CoV and SARS-related coronaviruses (CoVs), which share the same cellular receptor as SARS-CoV-2, also have threats to global public health. Therefore, it is critical to develop vaccines against multiple SARS-CoV-2 VOCs and other CoVs with pandemic potential. The spike (S) backbone region (with truncated RBD) of SARS-CoV-2 and its variants is highly conserved, albeit their RBDs are diversely different with many mutations. Herein, several subunit vaccines were designed by keeping only the conserved S backbone region of a SARS-CoV-2 Omicron subvariant (S-6P-no-RBD), or inserting the RBD of the Delta VOC (S-6P-Delta-RBD), the Omicron (BA5) VOC (S-6P-BA5-RBD), or the original SARS-CoV-2 (S-6P-WT-RBD) to the above construct, and their ability to induce neutralizing antibodies and protect against various SARS-CoV-2 VOCs and SARS-CoV were evaluated. Among the constructs tested, S-6P-Delta-RBD protein elicited broadly and potent neutralizing antibodies against all SARS-CoV-2 VOCs tested, including Alpha, Beta, Gamma, and Delta VOCs, BA1, BA2, BA2.75, BA4.6, and BA5 Omicron subvariants, as well as original strains of SARS-CoV-2 and SARS-CoV. This subunit vaccine significantly prevented SARS-CoV-2 Omicron infection and its replication, as well as completely protected against lethal challenge with the SARS-CoV-2 Delta VOC and SARS-CoV. S-6P-Delta-RBD vaccinated mouse sera potently prevented Delta variant infection, with significantly decreased lung viral titers and without showing pathological effects, and the protection positively correlated with the serum neutralizing antibody titer. Overall, the designed subunit vaccine holds promise for development as a universal COVID-19 subunit vaccine and a pan-coronavirus subunit vaccine to prevent current and future SARS-CoV-2 VOCs and SARS-related coronaviruses.

In the present example, several universal COVID-19 vaccines were designed that induced broadly and potent neutralizing antibodies against all SARS-CoV-2 VOCs tested (e.g., Alpha, Beta, Gamma, and Delta VOCs, BA1, BA2, BA2.75, BA4.6, and BA5 Omicron subvariants), and the original strain of SARS-CoV-2 or SARS-CoV. They also effectively protected immunized transgenic mice against SARS-CoV-2 Omicron challenge, and completely protected these mice against challenge of a lethal SARS-CoV-2 Delta variant and a lethal SARS-CoV. The related vaccine constructs can be designed in the format of subunit vaccines and mRNA vaccines. The designed vaccines have great potential to be further developed as universal COVID-19 vaccines and pan-coronavirus vaccines to prevent current and future SARS-CoV-2 VOCs and SARS-related coronaviruses. This approach can also be extended to design pan-coronavirus vaccines against not only SARS-CoV-2, SARS-CoV, and SARS-related coronaviruses, but also MERS-CoV and MERS-related coronaviruses, as well as other coronaviruses with pandemic potential in the future.

Coronavirus Disease 2019 (COVID-19), which is caused by severe acute respiratory syndrome coronavirus-2 (SARS-CoV-2), was first emerged in 2019, and has led a global pandemic with devastating economic losses and continuous severe threats to public health worldwide. SARS-CoV-2 has mutated frequently since its first emergency, and multiple mutations have been identified in the spike (S) protein, particularly the receptor-binding domain (RBD) fragment. These mutations have resulted in multiple variants of concerns (VOCs), which include Alpha, Beta, Gamma, Delta, and Omicron VOCs, whereas the Omicron VOC can be further classified as several subvariants, including BA1, BA2 (BA2.75), BA3, BA4 (BA4.6), BA5, and XBB1.5. The mutations in the S, particularly RBD, of SARS-CoV-2, have led to significant reduction or resistance of antibodies produced by the S protein or RBD of the original SARS-CoV-2 against these VOCs. Therefore, universal COVID-19 vaccines with potent neutralizing activity and protection against multiple current dominant and future SARS-CoV-2 VOCs with pandemic potential are urgently needed to prevent the current COVID-19 pandemic and future threats.

In addition to SARS-CoV-2, another coronavirus, SARS-CoV, which caused a global outbreak during 2002-2003, belongs to the same beta-coronavirus genus and uses the same cellular receptor as SARS-CoV-2 for viral entry. The potential of reoccurrence of SARS-CoV-induced SARS and other diseases potentially caused by SARS-related coronaviruses points out the importance of development of effective universal pan-coronavirus vaccines against not only SARS-CoV-2, but also SARS-CoV and other beta-coronaviruses with pandemic potential.

Among all proteins of SARS-CoV-2 and SARS-CoV, the surface spike (S) protein and its receptor-binding domain (RBD) fragment are critical targets for development of effective vaccines. The S protein consists of S1 and S2 subunits. The RBD in the S1 subunit of SARS-CoV-2 Omicron VOCs has significantly higher amino acid variations than the RBD of the earlier SARS-CoV-2 VOCs, including Delta; nevertheless, the backbone region (truncated RBD) of their S proteins is highly conserved. In this example, several subunit vaccines were designed based on the conserved backbone of S protein of SARS-CoV-2 Omicron BA1 variant (S-6P) (which contained the HexaPro sequences and a C-terminal Foldon trimerization domain and a His6 tag), with or without insertion of the respective RBD described below. Their ability to induce broadly neutralizing antibodies and cross-spectrum protective efficacy against multiple SARS-CoV-2 VOCs and SARS-CoV was evaluated. Their mechanisms of protection against SARS-CoV-2 infection were also elucidated.

### Results

Construction and characterization of universal subunit vaccines based on the S protein. Recombinant S plasmids were constructed to include only the backbone of the S protein of an Omicron BA1 subvariant by truncating its RBD (S-6P-no-RBD), or respectively inserting the RBD of 1) a Delta variant (S-6P-Delta-RBD), 2) an original SARS-CoV-2 wild-type (S-6P-WT-RBD), or 3) an Omicron BA5 variant (S-6P-BA5-RBD) into the above S backbone construct. The original S6P protein (without RBD truncation) was included as control.

S-6P-Delta-RBD subunit vaccine induced broadly and potent neutralizing antibodies against all SARS-CoV-2 VOCs and the original SARS-CoV-2 strain tested. The K18-human ACE2 (K18-hACE2) transgenic mice were inmmunized with each of the aforementioned proteins (10 μg/mouse), the Cocktail (combination of S-6P-Delta-RBD and S-6P-BA5-RBD, 5 μg/protein, 10 μg/mouse), or PBS control, plus adjuvants, and boosted twice with the same immunogens and adjuvants. Sera were collected 10 days after the third immunization, and detected for neutralizing activity against pseudotyped viruses expressing the S protein of SARS-CoV-2 Omicron subvariants, other VOCs, and the original SARS-CoV-2 and SARS-CoV.

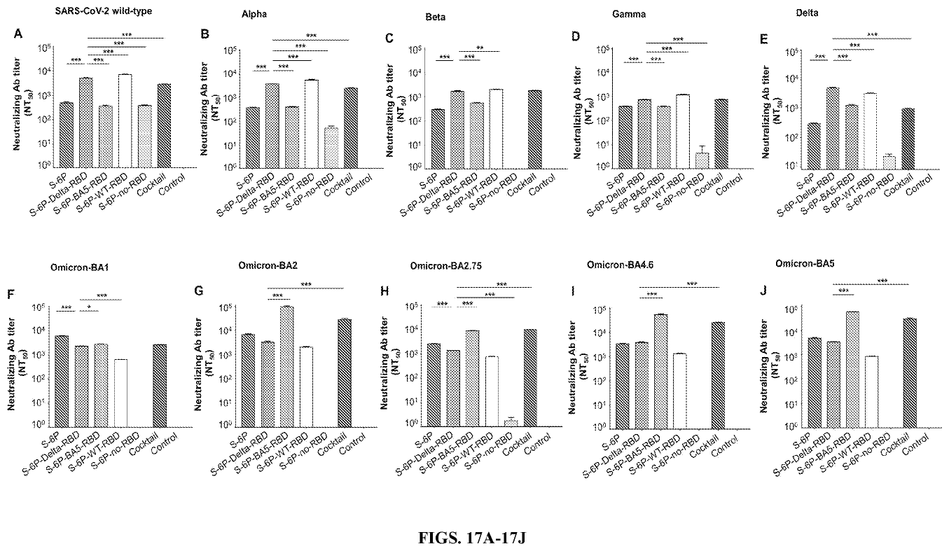

Notably, S-6P and S-6P-BA5-RBD proteins induced relatively low-titer neutralizing antibodies against the original SARS-CoV-2 and Alpha, Beta, Gamma, and Delta VOCs (FIGS. 17A, 17B, 17C, 17D, and 17E), although S-6P-BA5-RBD elicited potently higher neutralizing antibodies against five Omicron subvariants tested, including BA1, BA2, 2.75, BA4.6, and BA5 (FIGS. 17F, 17G, 17H, 17I, and 17J). By contrast, S-6P-Delta-RBD elicited broadly and potent neutralizing antibodies against all SARS-CoV-2 VOCs, including Alpha, Beta, Gamma, Delta, and five Omicron subvariants, as well as the original SARS-CoV-2 wild-type strain (FIGS. 17A, 17B, 17C, 17D, 17E, 17F, 17G, 17H, 17I, and 17J). The neutralizing antibody titers induced by S-6P-Delta-RBD were significantly higher than those induced by S-6P and S-6P-BA5-RBD proteins against Alpha, Beta, Gamma, and Delta SARS-CoV-2 VOCs and original strain (FIGS. 17A, 17B, 17C, 17D, and 17E), and were also significantly higher or higher than those induced by S-6P-WT-RBD against Omicron BA1, BA2, BA2.75, BA4.6, and BA5 subvariants (FIGS. 17F, 17G, 17H, 17I, and 17J). Unlike other subunit proteins, S-6P-no-RBD protein alone induced slightly neutralizing antibodies against pseudotyped SARS-CoV-2 wild-type strain and Alpha VOC, rather than against other SARS-CoV-2 pseudoviruses tested (FIGS. 17A, 17B, 17C, 17D, 17E, 17F, 17G, 17H, 17I, and 17J). Of note, combination of S-6P-Delta-RBD and S-6P-BA5-RBD proteins (Cocktail) induced highly potent neutralizing antibodies against all SARS-CoV-2 strains tested (FIGS. 17A, 17B, 17C, 17D, 17E, 17F, 17G, 17H, 17I, and 17J). However, mice injected with adjuvanted PBS control produced undetectable neutralizing antibodies against these pseudoviruses (FIG. 17).

The above data demonstrate that among all the single proteins tested, S-6P-Delta-RBD alone had ability to elicit balanced, potent, and broadly neutralizing antibodies against all SARS-CoV-2 pseudoviruses tested, with the antibody titers being comparable to those induced by the combination of S-6P-Delta-RBD and S-6P-BA5-RBD (Cocktail). Therefore, S-6P-Delta-RBD subunit vaccine has potential for development as a universal COVID-19 vaccine against current and future SARS-CoV-2 VOCs.

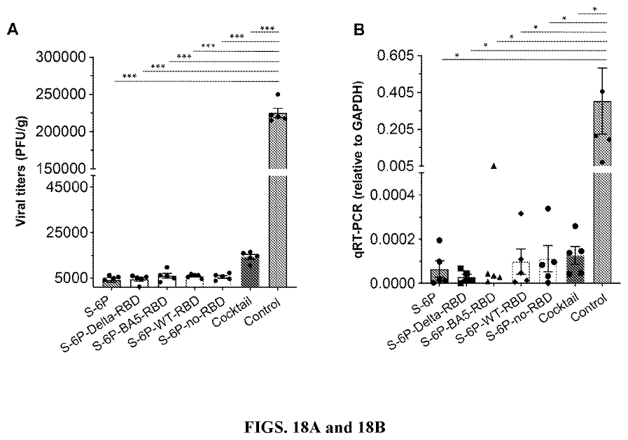

S-6P-Delta-RBD subunit vaccine completely protected immunized mice against SARS-CoV-2 Delta variant-induced weight loss and death. To evaluate the protective efficiency of designed subunit vaccines against lethal challenge with the SARS-CoV-2 Delta VOC, immunized K18-hACE2 mice were challenged with a high dose (10⁴ PFU/mouse) of this variant strain two weeks after the last vaccination, and their weights and overall survival were investigated subsequently for a period of 14 days. The control mice injected with adjuvanted PBS continuously lost weight, and all of them died by Day 10 after viral challenge (FIGS. 18A, 18B, 18C, 18D, 18E, 18F, 18G, 18H, 18I, 18J, and 18K). By comparison, all mice receiving a single protein, or their combination, survived the Delta variant infection during the detection period of 14 days (FIGS. 18F, 18G, 18H, 18I, 18J, and 18K). Particularly, no weight loss was observed in the mice immunized with S-6P-Delta-RBD alone, whereas obvious or significant weight loss was shown in the mice immunized with other single proteins, including S-6P, S-6P-no-RBD, S-6P-WT-RBD, and S-6P-BA5-RBD, or the combinatorial (cocktail) proteins of S-6P-Delta-RBD and S-6P-BA5-RBD (FIG. 18E). These data reveal that S-6P-Delta-RBD protein alone was sufficient to completely prevent Delta variant-induced death and weight loss.

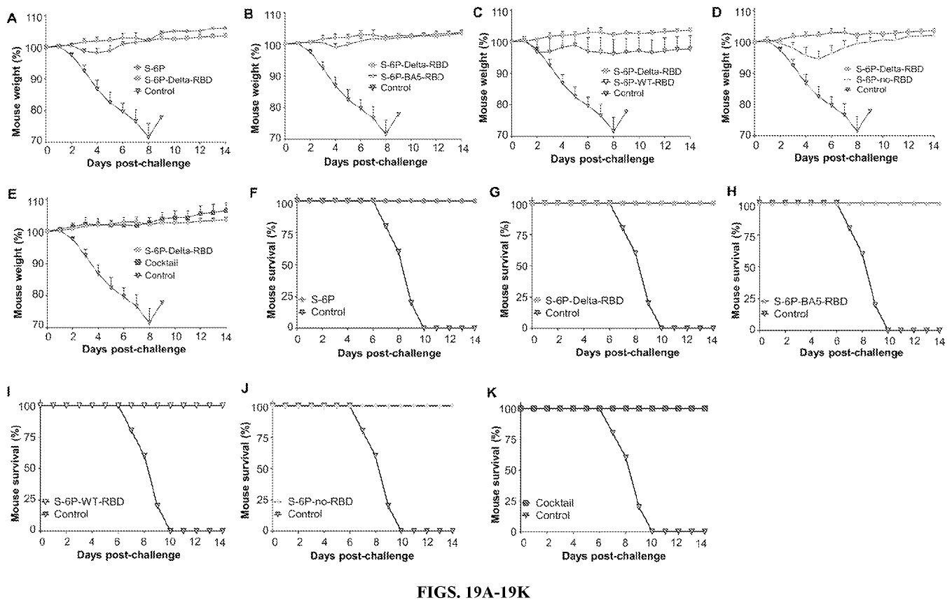

S-6P-Delta-RBD subunit vaccine protected immunized mice against SARS-CoV-2 Omicron variant infection and replication. The immunized K18-hACE2 mice were further evaluated for their protection against SARS-CoV-2 Omicron variant infection. The mice were challenged with the SARS-CoV-2 Omicron-BA1 two weeks after the last immunization, and viral titers and viral replication were detected in the lungs of challenged mice two days after viral infection. Evaluation of viral tiers indicated that the control mice receiving the adjuvanted PBS had the highest viral titers in their lungs, which were significantly higher than any other groups of the mice immunized with the test proteins (FIG. 19A). Particularly, the mice immunized with the S-6P-Delta-RBD protein, along with those immunized with the other single proteins, including S-6P, S-6P-WT-RBD, and S-6P-no-RBD, had the lowest viral titers in their lungs (FIG. 19A). Notably, mice receiving the combined S-6P-Delta-RBD and S-6P-BA5-RBD vaccines (Cocktail) had relatively higher viral titers than the other vaccination groups (FIG. 19A). Investigation of viral replication revealed that mice receiving the adjuvanted PBS control had significantly higher viral replication in their lungs than any vaccination groups (FIG. 19B). By contrast, mice immunized with the S-6P-Delta-RBD protein had the lowest viral replication in their lungs, followed by the mice immunized with the S-6P protein alone (FIG. 19B). These data indicate that S-6P-Delta-RBD alone was able to prevent SARS-CoV-2 Omicron variant challenge, showing significantly reduced viral titers and viral replication in the lungs of challenged mice. Therefore, this vaccine can be further developed as a universal COVID-19 vaccine against infection of SARS-CoV-2 Omicron and other variants.

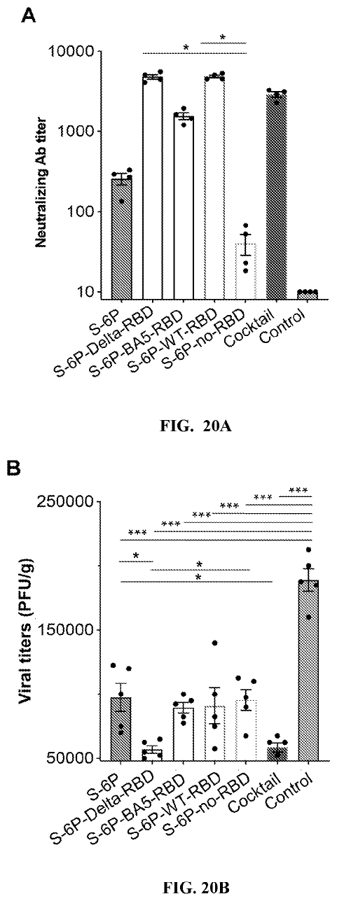

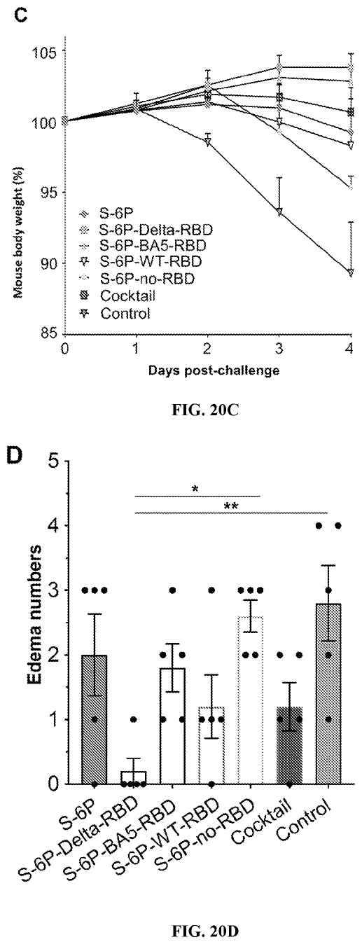

S-6P-Delta-RBD protein-immunized mouse sera protected naive mice against SARS-CoV-2 Delta variant infection without inducing pathological effects. To evaluate whether serum neutralizing antibodies from immunized mice play a critical role in preventing viral infection and virus-induced pathological effects, naïve K18-hACE2 mice were first injected with the mouse sera from each vaccination group, followed by infection with the SARS-CoV-2 Delta variant, and then investigation of viral titers and pathological changes in the lungs. The sera pooled from each group were measured for their neutralizing antibody titers before being injected into the mice, with those from the S-6P-Delta-RBD immunized mice showing the highest neutralizing antibodies titers, followed by those from the S-6P-Delta-RBD and cocktail immunized mouse sera (FIG. 20A). The data from the challenge studies revealed that viral titers in the mice receiving the S-6P-Delta-RBD immune sera were significantly lower than those in the other groups, and no body weight loss was observed in these mice after challenge (FIGS. 20B-20C). Of note, these serum neutralizing antibodies resulting from S-6P-Delta-RBD inmmunized mice effectively protected the recipient mice from virus-induced edema and inflammation (FIG. 20D). By contrast, immune sera from S-6P-no-RBD-vaccinated mice had the least or background-level neutralizing antibody titer (FIG. 20A). Thus, mice receiving these sera presented the higher viral titer in their lungs, constantly decreased weight after challenge, and showed greater number of edema than the groups receiving the other immune sera (FIGS. 20B, 20C, and 20D). Overall, the higher neutralizing antibody level in the transferred sera led to the lower viral titer in the lung (FIG. 20). These data demonstrate that serum neutralizing antibodies from the S-6P-Delta-RBD-immunized mice are critical in preventing recipient mice from the Delta variant challenge and the associated pathological effects, and that the protective efficacy positively correlated with the neutralizing antibody titer.

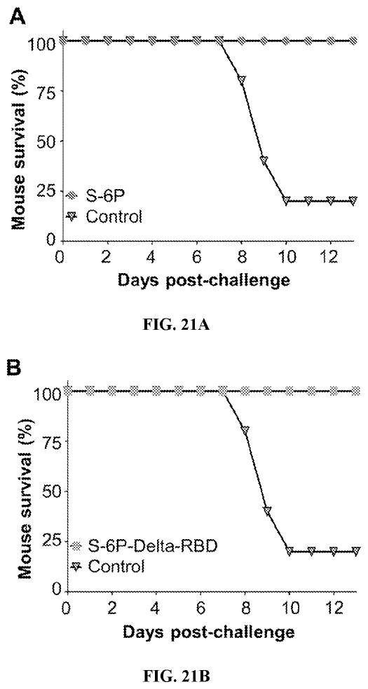

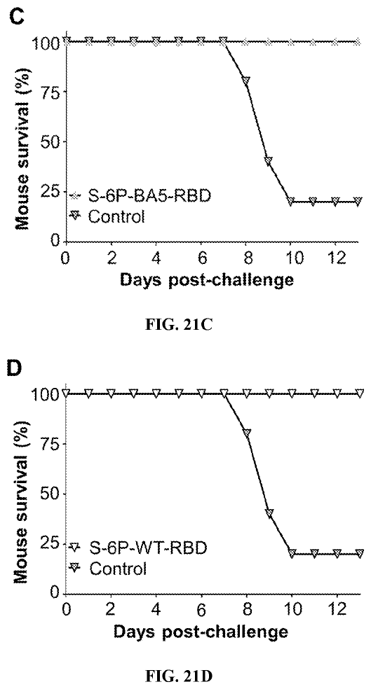

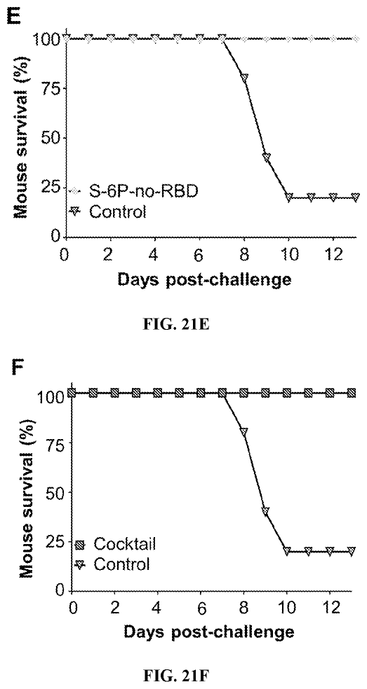

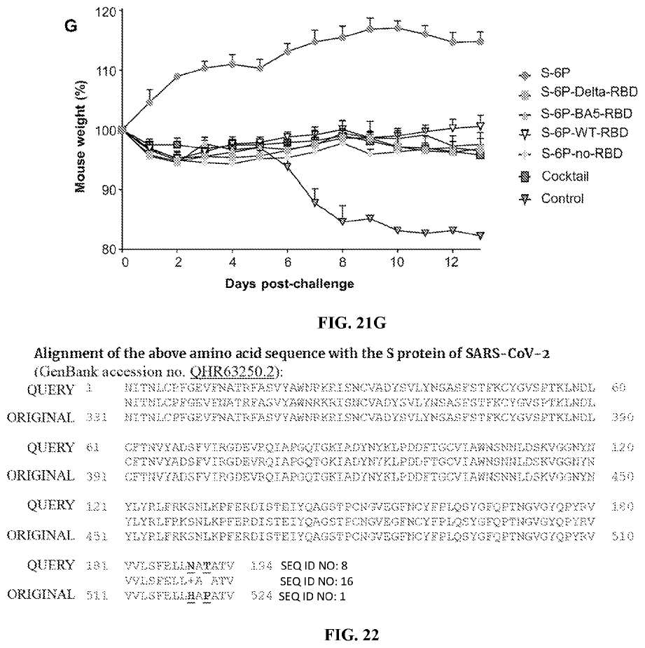

S-6P-Delta-RBD protein completely protected immunized mice against SARS-CoV-induced weight loss and death. To evaluate the protective efficacy of the designed subunit vaccines against SARS-CoV infection, immunized K18-hACE2 mice were challenged with a lethal dose (200 PFU/mouse) of SARS-CoV MA15 strain two weeks after the last immunization, followed by observation of the overall survival and weight changes after viral infection. 80% of the control mice receiving the adjuvanted PBS died by Day 10 after viral infection (FIGS. 21A, 21B, 21C, 21D, 21E, and 21F), which also presented constant weight loss (FIG. 21G). By contrast, all mice immunized with a single protein, including S-6P (FIG. 21A), S-6P-Delta-RBD (FIG. 21B), S-6P-BA5-RBD (FIG. 21C), S-6P-WT-RBD (FIG. 21D), or S-6P-no-RBD (FIG. 21E) protein, as well as all mice receiving the combined S-6P-Delta-RBD and S-6P-BA5-RBD proteins (Cocktail) (FIG. 21F), survived the SARS-CoV challenge, without significant weight loss ((FIGS. 21A, 21B, 21C, 21D, 21E, 21F, and 21G). These results indicate that single proteins alone, including S-6P-Delta-RBD, or its combination with the S-6P-BA5-RBD, completely protected vaccinated mice against SARS-CoV-induced death and weight loss after the challenge. Thus, these subunit vaccines, including S-6P-Delta-RBD, have potential to be developed as universal pan-coronavirus vaccines against SARS-CoV-2 and SARS-CoV.

### Methods

Preparation of vaccine antigens. The DNA sequence of SARS-CoV-2 Omicron BA1 variant containing a C-terminal foldon trimeric motif and His6 tag (S-6P) was amplified by PCR using a codon-optimized plasmid encoding the S protein of SARS-CoV-2 Omicron-BA1 with HexaPro sequences (a mutated furin cleavage site and six proline substitutions). The amplified PCR fragment was inserted into a pLenti mammalian cell expression vector. The S-6P-no-RBD was constructed by truncating the RBD fragment of the above S-6P sequence using a seamless cloning kit. The S-6P-Delta-RBD, S-6P-BA5-RBD, S-6P-WT-RBD, and S-6P-SARS-RBD were constructed by replacing the RBD of S-6P sequence with the respective RBD fragment of SARS-CoV-2 Delta variant, Omicron-BA5 subvariant, or the original strains of SARS-CoV-2 using the seamless cloning kit. Each recombinant plasmid was confirmed for correct sequences, and transfected into HEK293F cells, followed by purification of proteins from the cell culture supernatants using Ni-NTA Superflow (Qiagen).

Coronavirus pseudovirus preparation and neutralization assay. Recombinant plasmids encoding the S protein of SARS-CoV-2 original wild-type (WT) strain (GenBank accession number QHR63250.2), SARS-CoV original strain (GenBank accession number AY274119), and SARS-CoV-2 Alpha variant (GISAID accession number EPI_ISL_718813) were constructed by inserting each DNA sequence into a pcDNA3.1/V5-His-TOPO vector. SARS-CoV-2 Omicron BA1 (GISAID accession number EPI_ISL_6795835), BA2 (GISAID accession number EPI_ISL_12030355), BA2.75 (GISAID accession number EPI_ISL_14384334), BA4.6 (GISAID accession number EPI_ISL_14288784), and BA5 (GISAID accession number EPI_ISL_12043290), as well as recombinant plasmids expressing each S protein of Beta, Gamma, and Delta variants, were constructed using a multi-site-directed mutagenesis kit, and the mutations in the RBD region were included in each construct. The sequence-confirmed plasmids were used for generation of pseudoviruses as described below.

Pseudovirus generation and neutralization assay. Pseudoviruses were generated by co-transfecting each recombinant plasmid with pLenti-CMV-luciferase and PS-PAX2 plasmids into 293T cells using a PET transfection method. Pseudovirus-containing culture supernatants were collected 72 h after transfection and incubated with serially diluted mouse sera at 37° C. for 1 h. The virus-serum mixture was then added to hACE2/293T cells. 24 h later, fresh medium was added to the cells, which were further cultured for 48 h. The cells were then sequentially incubated with cell lysis buffer and luciferase substrate, and then measured for relative luciferase activity using Cytation 7 Microplate Multi-Mode Reader and Gen5 software. Pseudovirus neutralization was calculated as 50% neutralizing antibody titer (NT₅₀).

Ethics statement. Male and female K18-hACE2-transgenic (Tg) mice were utilized in this study. The animal protocols were approved by the Institutional Animal Care and Use Committees (IACUC). All mouse-related experiments were conducted by strictly following approved protocols and the Guidelines for the Care and Use of Laboratory Animals of National Institutes of Health.

Animal immunization and serum collection. Mice were intramuscularly (I.M.) immunized with each protein (10 μg/mouse) or PBS control plus aluminum (500 μg/mouse) and monophosphoryl lipid A (MPL, 10 μg/mouse) adjuvants (InvivoGen). Cocktail is the combination of S-6P-Delta-RBD and S-6P-BA5-RBD (5 μg/protein, 10 μg/mouse). These mice were further boosted twice with the same immunogen and adjuvants every 3 weeks. The sera were collected from each mouse 10 days after the last immunizations and measured for neutralizing antibodies against SARS-CoV-2 and SARS-CoV, as described above.

Virus challenge studies. Two weeks after the last immunization, mice were intranasally (I.N.) infected with the following viruses. 1) SARS-CoV-2 Omicron BA1 variant (10⁵ plaque forming unit: PFU/mouse, 50 μl/mouse). The mice were sacrificed two days after the infection, and lungs were collected for measurement of viral titers and viral replication. 2) SARS-CoV-2 Delta variant (10⁴ PFU/mouse, 50 μl/mouse). The mice were observed for survival and body weight changes for 14 days after virus challenge. 3) SARS-CoV MA15 strain (200 PFU/mouse, 50 μl/mouse). The mice were observed for survival and body weight changes for 13 days after virus infection.

Viral titer detection. Lung tissues collected from SARS-CoV-2-challenged mice were measured by a plaque assay. Specifically, lung tissues were homogenized and centrifuged, and the supernatants were diluted in DMEM cell culture medium, followed by incubation with Vero E6 cells for 1 h at 37° C. The inoculum was removed; the cells were then overlaid with 0.6% agarose and cultured for three days. After removing the overlays, the cells were stained with 0.1% crystal violet to show plaques. Viral titers were calculated as PFU/g of mouse lung tissue.

Viral RNA detection by qPCR. RNA was extracted from SARS-CoV-2-infected mouse lungs using TRIzol reagent by Invitrogen according to the manufacturer's instructions. 1 μg of total RNA was used as template for the first strand of cDNA. The resulting cDNA was subjected to amplification of selected genes by real-time quantitative PCR (qPCR) using Power SYBR Green PCR Master Mix. Viral nucleocapsid (N) gene was detected using nCOV_N1 primer. The expression levels were normalized to glyceraldehyde-3-phosphate dehydrogenase (GAPDH) by the following threshold cycle (CT) equation: ΔCT=CT of the gene of interest−CT of GAPDH. All results are shown as a ratio to GAPDH calculated as 2^{-ΔCT}.

Challenge of naïve mice receiving immune sera with SARS-CoV-2 Delta variant. The naive mice were intraperitoneally (I.P.) administered with the pooled mouse sera (200 μl/mouse) from different immunization groups. Six hours after serum administration, the mice were I.N. infected with SARS-CoV-2 Delta variant (5×10³ PFU/mouse, 50 μl/mouse). Four days after viral infection, the mice were euthanized for lung collection. Half lungs were collected in PBS for measurement of viral titers by plaque assay, and the other half were collected in Zinc formalin for pathological analysis, as described below.

Pathological analysis of lung tissues. Lungs collected from SARS-CoV-2 Delta variant-challenged mice were analyzed for pathological effects. Specifically, paraffin-embedded lung tissue sections were stained using the hematoxylin and eosin (HE) method, and the relevant slides were examined for pathological effects using a grouped masking approach. Edema and interstitial inflammation distribution in the lungs were ordinally numbered, with the scores being reported as 0 (none), 1 (<25%), 2 (26-50%), 3 (51-75%), and 4 (>75%) of tissue fields.

Statistical data analysis. GraphPad Prism 9 statistical software was used to calculate statistical significance among various vaccinations groups. Ordinary one-way ANOVA (multiple comparison test) was performed to assess statistical significance of vaccine-induced antibody or neutralizing antibody titers, as well as viral titers, viral replication, and pathological changes in the lung tissues of challenged mice. Dunn's multiple comparison test was performed to analyze statistical significance of neutralizing antibody titers of pooled mouse sera for passive transfer. *, **, and *** designate P<0.05, P<0.01, and P<0.001, respectively.

### Example 5. Glycosylated Delta-RBD Mucosal Vaccine with or without Omicron-Spike Protein Priming Elicits Broadly Neutralizing Antibodies with Protection Against SARS-CoV-2 Challenge

Because SARS-CoV-2 is a mucosal pathogen, mucosal COVID-19 vaccines need to be developed to block viral infection at the mucosal site. Herein, a mucosal vaccine was designed based on a glycosylated receptor-binding domain of Delta variant (Delta-RBD). The intranasal (i.n.) delivery of Delta-RBD protein elicited potent and balanced systemic antibody responses with high antibody titers, which were comparable to those induced by intramuscular (i.m.) injection of the same vaccine or the Omicron-S subunit vaccine. It also induced significantly higher mucosal IgA antibody levels than the other modes of vaccination. Moreover, this mucosal vaccine induced broadly neutralizing antibodies against the original SARS-CoV-2 strain, and the Delta and Omicron BA1/BA2 variants, completely protecting mice transgenic for the human angiotensin-converting enzyme 2 against lethal challenge with the SARS-CoV-2 Delta variant, including complete absence of weight loss. Of note, i.m. priming with the Omicron-S protein followed by i.n. boosting with the Delta-RBD protein further improved the vaccine's ability to generate broad-spectrum neutralizing antibodies against the recent BA5 and XBB Omicron variants. Overall, mucosal vaccine prevents SARS-CoV-2 infection of the respiratory mucosa, while the i.m. priming and i.n. boosting vaccination strategy offers protection against known and emerging SARS-CoV-2 variants.

The global Coronavirus Disease 2019 (COVID-19) pandemic, caused by severe acute respiratory syndrome coronavirus-2 (SARS-CoV-2), has led to extensive economic damage and devastating loss of human life. Although SARS-CoV-2 has a much lower mortality rate than SARS-CoV and Middle East respiratory syndrome (MERS)-CoV, which caused the 2002 epidemic and 2012 outbreaks, respectively, it has much broader and long-lasting consequence. As of May 31, 2023, SARS-CoV-2 has infected at least 767 million people and caused over 6.9 million deaths.

The SARS-CoV-2 genome encodes four structural proteins, namely the spike (S), envelope, nucleocapsid, and membrane proteins, among which the surface S protein plays a critical role in viral infection. The S protein of SARS-CoV-2 is composed of the S1 and S2 subunits. Viral infection is initiated by binding of the receptor-binding domain (RBD) in the S1 subunit to its cellular receptor, angiotensin-converting enzyme 2 (ACE2). Thus, the S protein, and specifically its RBD fragment, are important targets for development of effective COVID-19 vaccines. Multiple SARS-CoV-2 variants have been characterized to date, including Alpha, Beta, Gamma, Delta, and Omicron (as well as its subvariants). Although the available COVID-19 vaccines targeting the S protein and/or its RBD exhibit high potency against the earlier SARS-CoV-2 strains and variants, they fail to induce broad-spectrum neutralizing antibodies and therefore high levels of protection against the currently dominant SARS-CoV-2 Omicron variants.

On May 5, 2023, the World Health Organization (WHO) declared that COVID-19 was no longer a Public Health Emergency of International Concern. Additionally, the United States COVID-19 public health emergency came to an end on May 11, 2023, thereby concluding the 3-year COVID-19 pandemic. However, the challenge of preventing deaths from the high transmissible and mutation-prone SARS-CoV-2 remains. Therefore, the development of effective vaccines to prevent infection with current and future SARS-CoV-2 variants with pandemic potential is still a priority.

The immunoglobulin (Ig)G Fc fragment have been used as a vehicle for the effective intranasal delivery of vaccines to mucosal surfaces, and subsequent induction of mucosal and systemic immune responses against respiratory viral pathogens such as MERS-CoV, respiratory syncytial virus, and influenza virus. Because SARS-CoV-2 is a respiratory viral pathogen, the mucosal or intranasal (i.n.) delivery of COVID-19 vaccines (i.e., via the IgG Fc fragment) elicit both local and systemic immune responses with superior levels of protection than conventional intramuscular (i.m.) vaccines.

Example 2 showed that glycosylation of amino acid residues 519 and 521 within the RBD of the original SARS-CoV-2 strain masked a non-neutralizing epitope containing residue 519 and induced significantly higher systemic neutralizing antibody responses than the non-glycosylated form of the RBD; this led to superior protection against infection with the original SARS-CoV-2 strain. In the present example, a mucosal COVID-19 vaccine was designed by fusing a glycosylated Delta variant RBD to the Fc fragment of human IgG, and evaluated for its ability to elicit systemic and mucosal immune responses, as well as protect K18-human (h)ACE2 transgenic model mice against lethal SARS-CoV-2 challenge. In addition, it was determined how different routes of administration affected the broadly neutralizing activity and protective efficacy of the glycosylated Delta-RBD mucosal vaccine and a trimeric S protein of SARS-CoV-2 Omicron variant (Omicron-S)-based vaccine. Finally, it was assessed whether the neutralizing activity and protection of the mucosal vaccine is improved by priming with Omicron-S subunit vaccine.

### Materials and Methods

Preparation of recombinant protein vaccines. The glycosylated Delta-RBD DNA construct containing a C-terminal Fc tag of human IgG1 was prepared by mutation of residues L452R and T478K of the original RBD sequence (with glycosylation of residues 519 and 521 of the S protein) of SARS-CoV-2 strain (GenBank accession number QHR63250.2) using a multi-site-directed mutagenesis kit (Agilent Technologies), and insertion into a pLenti expression vector. The Omicron-S DNA construct contained the codon-optimized S sequence of B.1.1.529 (Omicron) variant (GISAID accession number EPI_ISL_6795835) of SARS-CoV-2, and HexaPro sequences, as well as a C-terminal foldon trimerization motif and a His6 tag, which was then inserted into the above vector. The relevant RBD residues of this construct were mutated to include additional substitutions at residues S371F, T376A, D405N, R408S, L452R, and F486V (whereas residues G446, Q493, and G496 were not mutated). The sequence-confirmed recombinant DNA plasmids were transfected into HEK293F cells, and related proteins were purified from culture supernatants using nProtein A Sepharose 4 Fast Flow (for Delta-RBD) (GE Healthcare) and Ni-NTA Superflow (for Omicron-S) (Qiagen), respectively.

Enzyme-linked immunoassay. Enzyme-linked immunoassay (ELISA) was used to test specific IgG and IgA antibodies induced in immunized mouse sera and lung BAL fluid, respectively. Specifically, ELISA plates were respectively coated with the Delta-RBD and Omicron-S proteins (1 μg/ml) at 4° C. overnight, and blocked with the PBST-blocking buffer (2% non-fat milk in PBS containing 0.05% Tween-20) at 37° C. for 2 h. The plates were washed with PBST buffer for five times, and then sequentially incubated with mouse sera at serial dilutions or lung BAL fluid collection, and horseradish peroxidase (HRP)-conjugated anti-mouse IgG (1:5,000, Sigma) or anti-mouse IgA (1:5,000, Sigma) antibody at 37° C. for 1 h. After further washes, the plates were incubated with TMB (3,3′,5,5′-Tetramethylbenzidine) substrate (Sigma), followed by stop of the reaction by H₂SO₄ (1 N). Absorbance at 450 nm (A450 value) was measured using Cytation 7 Microplate Multi-Mode Reader (BioTek Instruments).

SARS-CoV-2 pseudovirus generation and pseudovirus neutralization assay. These experiments were performed as described below. For pseudovirus generation, a recombinant wild-type (WT)-S-DNA plasmid encoding S protein of the original SARS-CoV-2 strain (GenBank accession number QHR63250.2) was constructed by inserting the S gene sequence into pcDNA3.1/V5-His-TOPO vector (Thermo Fisher Scientific). The S-DNA plasmids of Delta variant and Omicron subvariants, including BA1 (GISAID accession number EPI_ISL_6795835), BA2 (GISAID accession number EPI_ISL_12030355), BA5 (GISAID accession number EPI_ISL_12043290), BQ1.1 (GISAID accession number EPI_ISL_15370776), and XBB (GISAID accession number EPI_ISL_15341139), were constructed to contain the respective mutations at the RBD using the multi-site-directed mutagenesis kit. The sequence-confirmed recombinant plasmids were used for subsequent generation of pseudoviruses. Each of the above plasmids was respectively co-transfected with PS-PAX2 and pLenti-CMV-luciferase plasmids into 293T cells using the PEI transfection method, followed by collection of pseudovirus-containing supernatants 72 h post-transfection. For pseudovirus neutralization assay, each pseudovirus was respectively incubated with serially diluted mouse sera at 37° C. for 1 h, followed by addition of the virus-serum mixture to hACE2/293T cells pre-seeded in 96-well plates. After addition of fresh medium 24 h later, the cells were continually cultured for up to 72 h, and lysed using cell lysis buffer, followed by incubation with luciferase substrate (Promega). Relative luciferase activity was then measured using Cytation 7 Microplate Multi-Mode Reader. 50% neutralizing antibody titer (NT₅₀) of each serum sample was calculated accordingly.

Ethical statement. Male and female C57BL/6 (B6) mice and female K18-hACE2 transgenic mice (6-8-week-old) were used for the immunization studies. They were randomly assigned to different vaccination groups. The animal protocols were approved by the Institutional Animal Care and Use Committees (IACUC). All mouse experiments were performed according to approved protocols and the Guidelines for the Care and Use of Laboratory Animals of the National Institutes of Health.

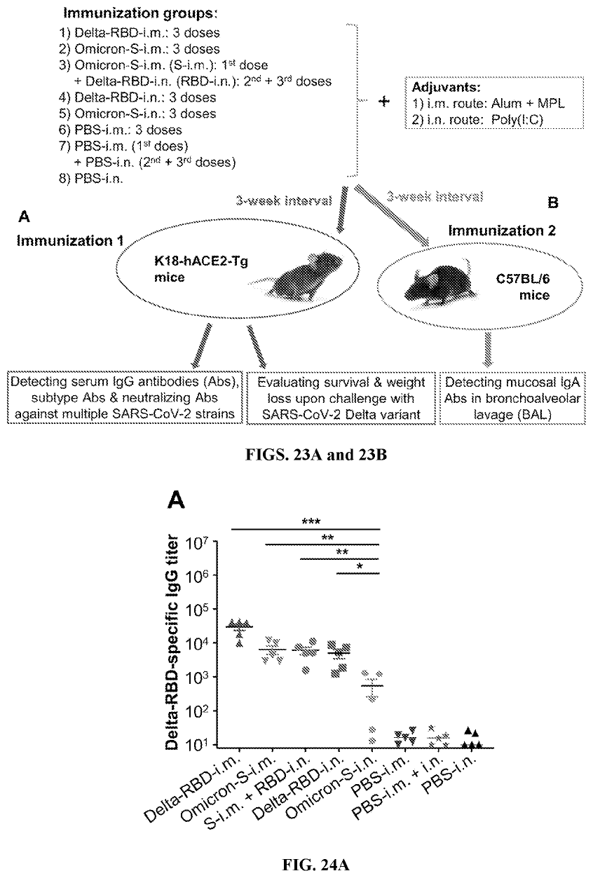

Immunization procedures and sample collection. Two separate immunizations were performed to evaluate immune responses, neutralizing antibodies, and protective efficacy induced by the vaccines (FIG. 23). First, K18-hACE2 mice (B6 background) were immunized with the following proteins (10 μg protein/mouse) or controls: intramuscularly (i.m.) with 1) Delta-RBD or 2) Omicron-S for three doses at 3 weeks, 3) i.m. with Omicron-S for the 1^{st} dose and intranasally (i.n.) with Delta-RBD for two boosts at 3 weeks, and i.n. with 4) Delta-RBD or 5) Omicron-S for three doses at 3 weeks. PBS injected via i.m., i.m.+i.n., and i.n. routes, respectively, was included as controls. Aluminum (500 μg/mouse) and monophosphoryl lipid A (MPL, 10 μg/mouse) adjuvants (InvivoGen) were used for proteins via the i.m. route (100 N1/mouse), and Poly(I:C) (10 μg/mouse) adjuvant (InvivoGen) was used for proteins via the i.n. route (20 μl/mouse). The use of the above adjuvants for the i.m. and i.n. routes were based on previously optimized protocols. Sera were collected 10 days after the last immunization for detection of specific IgG and neutralizing antibodies as described above. Immunized mice were challenged with the SARS-CoV-2 Delta variant four weeks after the last immunization for evaluation of protective efficacy. Second, B6 mice were immunized with each protein or control as described above. Four weeks after the last immunization, BAL fluid was collected from mouse lungs for evaluation of specific IgA antibody response as described above.

Challenge of mice with SARS-CoV-2 Delta variant. The challenge experiments were performed as described below. Four weeks after the last immunization, K18-hACE2 mice were i.n. challenged with a Delta variant of SARS-CoV-2 (10⁴ plaque-forming unit (PFU)/mouse, 50 μl/mouse), and investigated for body weight changes and survival for 14 days post-infection. Mice that were moribund or lost 30% weight were humanely euthanized by cervical dislocation under anesthesia.

Statistical analysis. Statistical significance among different groups was determined using statistical software (GraphPad Prism 9). Statistical differences of IgG and IgG subtype antibody responses, as well as neutralizing antibody titers, among different groups were calculated using Tukev's multiple comparison test. Statistical differences of weight changes among different groups after viral challenge were compared using unpaired two-tailed student t test. *, **, and *** indicate P<0.05, P<0.01, and P<0.001, respectively.

### Results

Intranasal Delivery of Glycosylated Delta-RBD with or without Omicron-S (i.m.) Priming Induced Potent and Balanced Systemic Antibody Responses

To evaluate the ability of the glycosylated Delta-RBD mucosal subunit vaccine to induce systemic immune responses, K18-hACE2 (C57BL/6 (B6) background) mice were i.n. immunized with glycosylated Delta-RBD (Delta-RBD-i.n.) conjugated to the mucosal adjuvant polyinosinic-polycytidylic acid (Poly(I:C)) (FIG. 23). Control mice were immunized with 1) Delta-RBD via i.m. (Delta-RBD-i.m.), 2) Omicron-S via i.m. (Omicron-S-i.m.), 3) Omicron-S via i.n. (Omicron-S-i.n.), or 4) Omicron-S via i.m. priming and then Delta-RBD via i.n. boosting (Delta-RBD-i.n.+Omicron-S-i.m.). Phosphate-buffered saline (PBS) was used as the vehicle in all vaccination experiments. Previously optimized Alum and MPL combinations or Poly(I:C) were used as adjuvants for i.m. and i.n. immunizations, respectively. There mice were boosted two times using the same immunogens, and their sera were collected 10 days after the last immunization to detect SARS-CoV-2-specific IgG and IgG subtype antibodies (FIG. 23A).

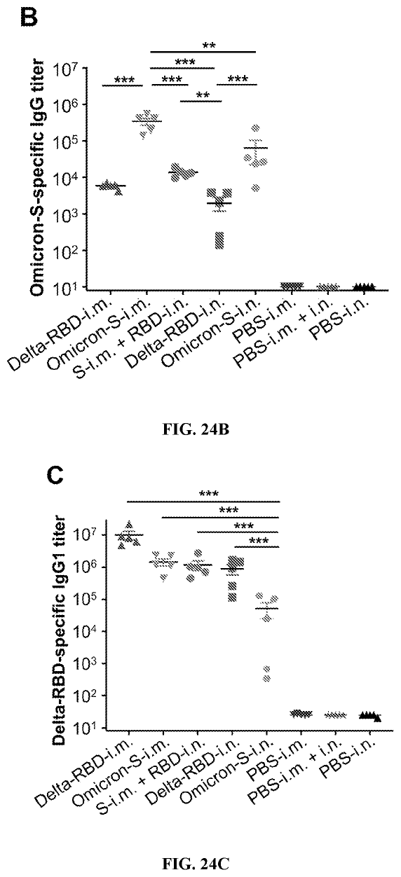

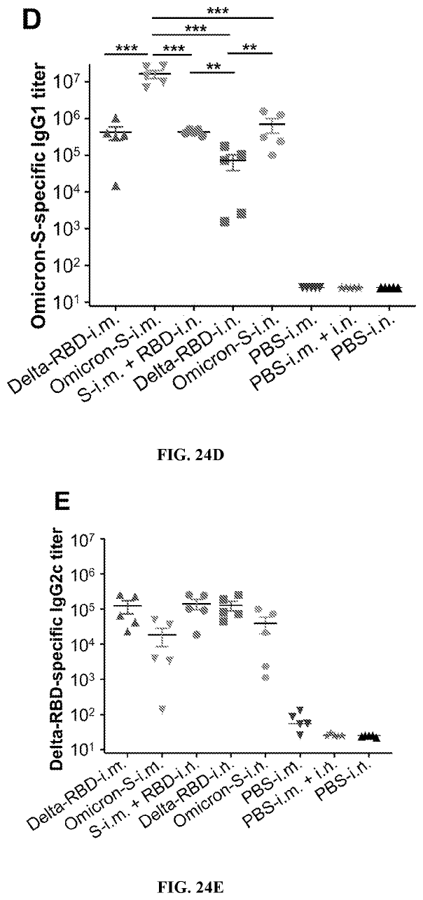

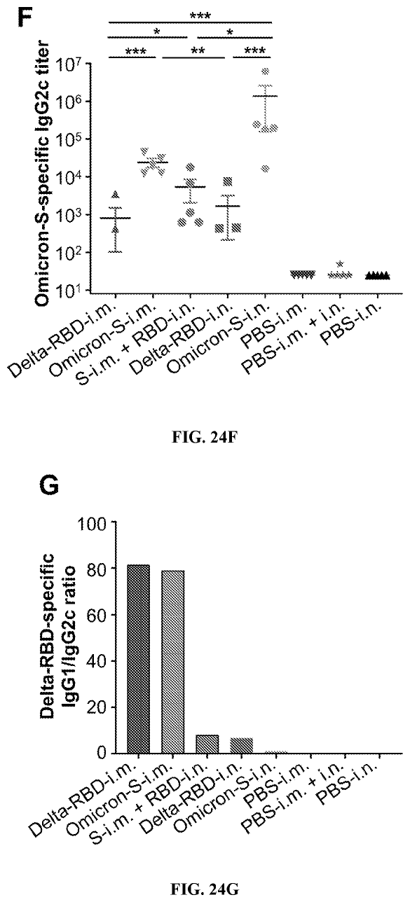

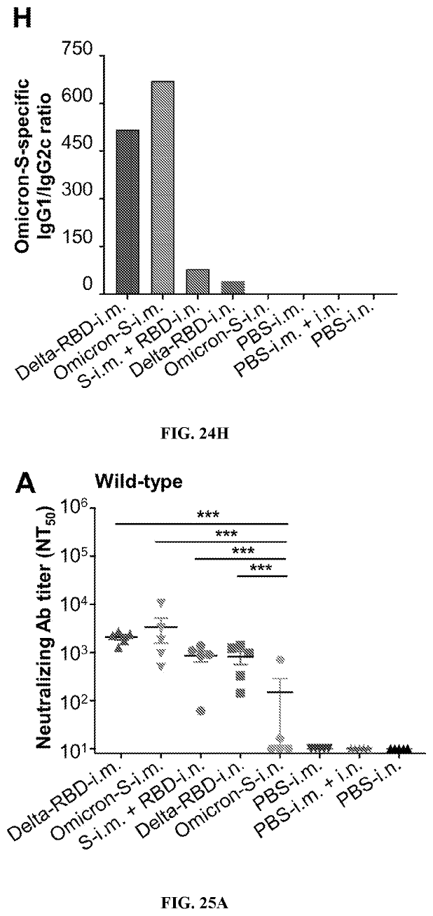

Delta-RBD-i.n. was found to induce a potent IgG antibody response against the Delta-RBD protein, which was comparable to that induced by Delta-RBD-i.m. or Omicron-S-i.m., and Omicron-S-i.m.+Delta-RBD-i.n. priming-boosting, but significantly higher than that elicited by Omicron-S-i.n. alone (FIG. 24A). Immunization with Delta-RBD-i.n. also elicited cross-reactive IgG antibodies against the Omicron-S protein, with the antibody titer being similar to that elicited by Delta-RBD-i.m.; however, the antibody titer following Delta-RBD-i.n. vaccination was significantly lower than that induced by immunization with Omicron-S-i.m. or i.n. (FIG. 24B). Of note, the anti-Omicron-S IgG antibody titer induced by i.n. immunization with Delta-RBD after Omicron-S-i.m. priming was significantly higher than that elicited by vaccination with Delta-RBD-i.n. alone (FIG. 24B). Immunization with Delta-RBD-i.n. also elicited a potent IgG1 subtype antibody response, with strong reactivity against Delta-RBD and cross-reactivity against Omicron-RBD; moreover, these antibodies were present at a similar trend to, but much higher levels than, those of the IgG antibodies targeting both proteins (FIGS. 24A, 24B, 24C, and 24D). In addition, the Delta-RBD-i.n. vaccination induced IgG2c subtype antibodies, which potently recognized Delta-RBD protein and cross-reacted with Omicron-S protein (FIGS. 24E and 24F). By contrast to the i.m. immunization route, the i.n. delivery of Delta-RBD or Omicron-S induced similar levels of IgG1 and IgG2c antibodies (corresponding to low IgG1/IgG2c ratios) targeting both Delta-RBD and Omicron-S proteins (FIGS. 24G and 24H). These data indicate that mucosal immunization with the Delta-RBD vaccine alone was sufficient to induce strong and balanced systemic antibody responses.

### Intranasal Delivery of Glycosylated Delta-RBD, Particularly Omicron-S (i.m.) Priming and Glycosylated Delta-RBD (i.n.) Boost, Induced Broadly Anti-SARS-CoV-2 Neutralizing Antibodies

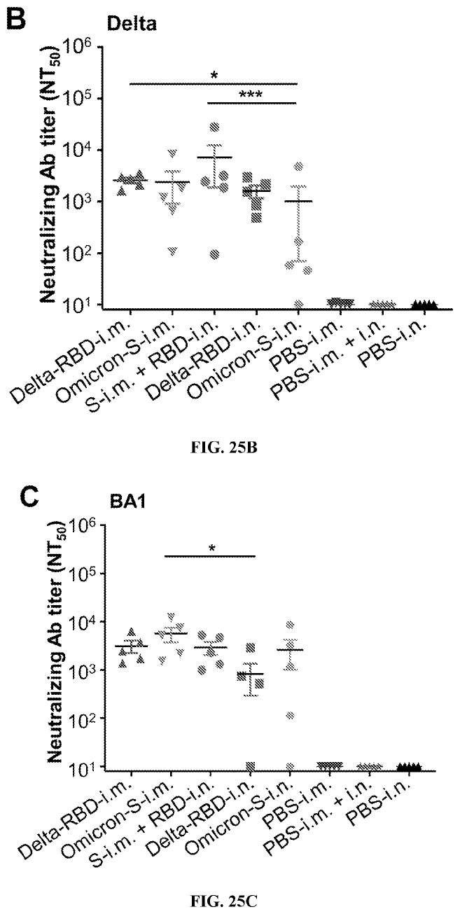

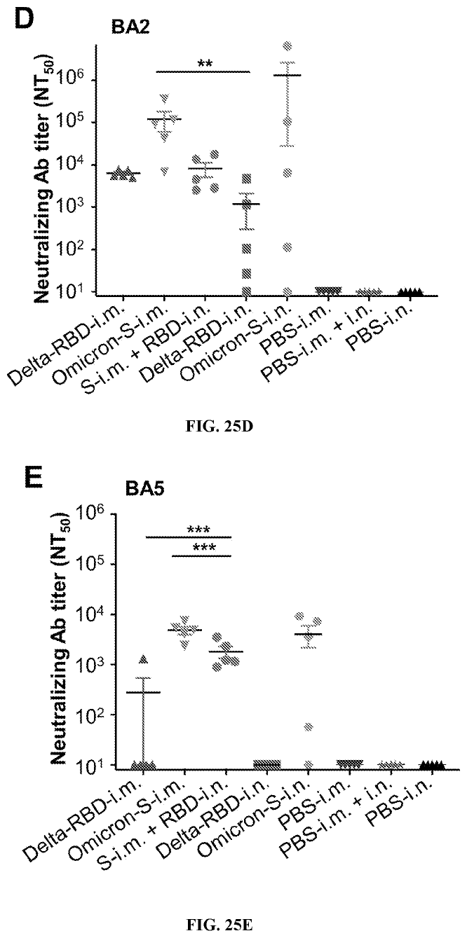

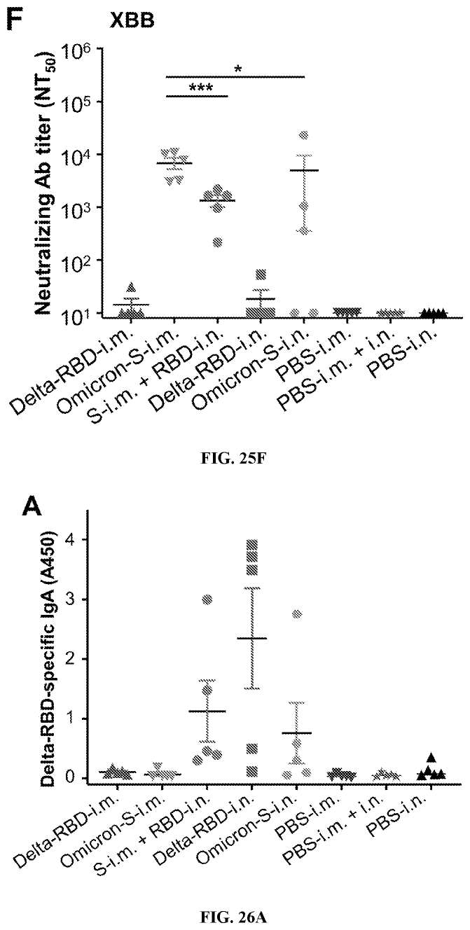

Next, the neutralizing and cross-neutralizing abilities of the glycosylated Delta-RBD mucosal vaccine against the pseudotyped original strain (wild-type) of SARS-CoV-2 and its multiple variants were tested using mouse sera collected from the aforementioned immunization experiments (FIG. 23A). Immunization with Delta-RBD-i.n., with or without Omicron-S-i.m. priming, elicited effective neutralizing antibodies against infection with the wild-type and Delta variant pseudoviruses (FIGS. 25A and 25B). These neutralizing antibody titers were comparable to those induced by i.m. immunization with Delta-RBD or Omicron-S, but were significantly higher, or higher, than those induced by i.n. immunization with Omicron-S. Delta-RBD-i.n. vaccination also induced cross-neutralizing antibodies against the Omicron BA1 and BA2 pseudoviruses (FIGS. 25C and 25D). Moreover, Delta-RBD-i.n. boosting after Omicron-S-i.m. priming further increased neutralizing antibody titers against Omicron BA2, while also inducing an effective neutralizing antibody response against the Omicron BA5 and XBB pseudoviruses (FIGS. 25E and 25F). Overall, these data indicate that i.n. immunized with Delta-RBD alone induced broadly neutralizing antibodies against the original strain of SARS-CoV-2 and the Delta and Omicron (BA1 and BA2) variants, whereas i.m. priming with Omicron-S followed by Delta-RBD-i.n. boosting elicited potent neutralizing antibodies against all SARS-CoV-2 strains tested.

### Intranasal Delivery of Glycosylated Delta-RBD or Omicron-S Protein Induced Strong Mucosal IgA Antibody Responses

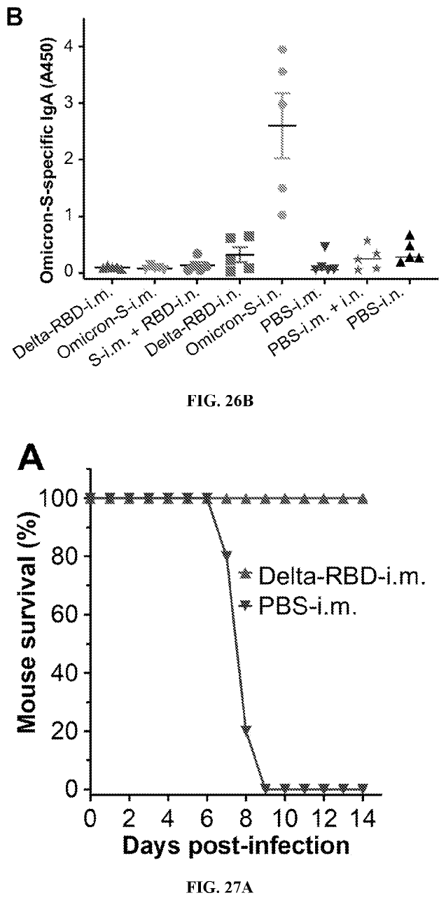

To evaluate the ability of glycosylated Delta-RBD to induce a mucosal immune response, B6 mice were i.n. immunized with this protein or the respective controls. Bronchoalveolar lavage (BAL) fluid was collected from each mouse 1 month after the last immunization (the time point used for the SARS-CoV-2 challenge, as described below) for specific secretory IgA antibody detection (FIG. 23B). The delivery of Delta-RBD-i.n. alone elicited high-levels of IgA antibodies specific to Delta-RBD, which showed weaker or background levels of cross-reactivity with Omicron-S (FIGS. 26A and 26B). Interestingly, the i.n. delivery of Omicron-S alone also induced adequate levels of anti-Omicron-S IgA antibodies (FIG. 26B). By contrast, i.m. immunization with Delta-RBD or Omicron-S protein only elicited background levels of IgA antibodies specific to both proteins, which was similar to those induced in the control mice receiving PBS and adjuvant (FIGS. 26A and 26B). These results reveal that Delta-RBD or Omicron-S via the i.n., but not the i.m., route elicits potent mucosal anti-SARS-CoV-2 antibody responses.

Intranasal Delivery of Glycosylated Delta-RBD Completely Protected Mice from Lethal Delta Variant Challenge without Obvious Weight Loss

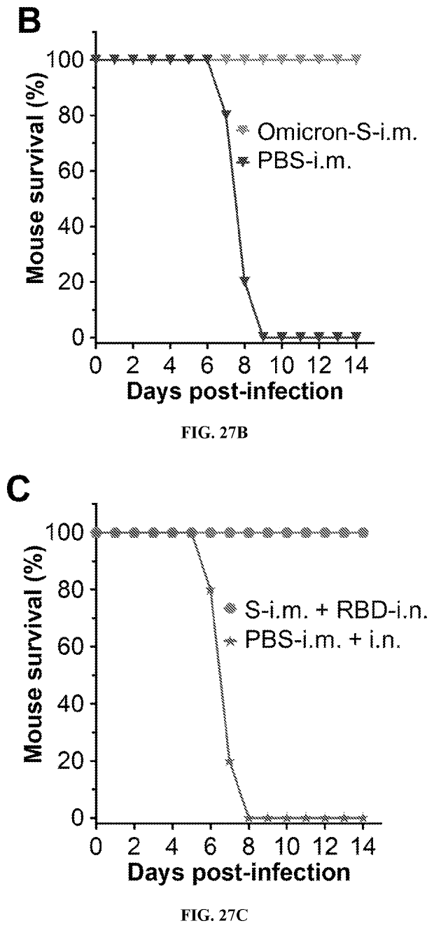

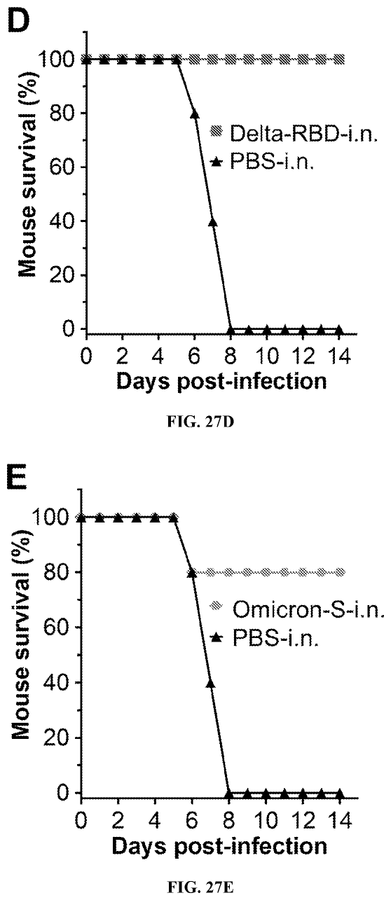

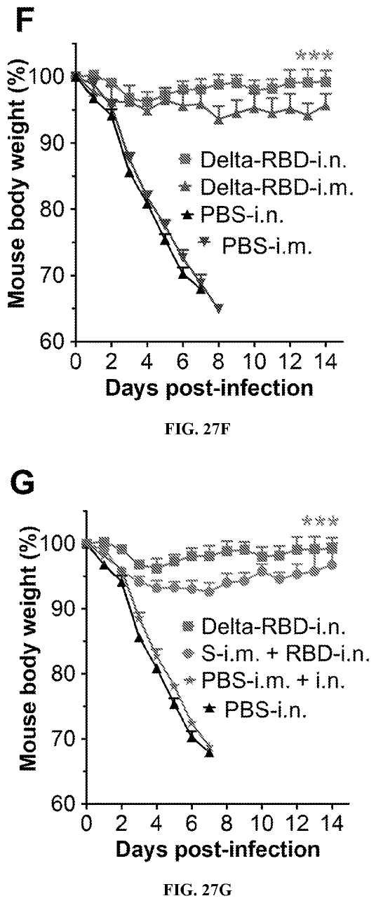

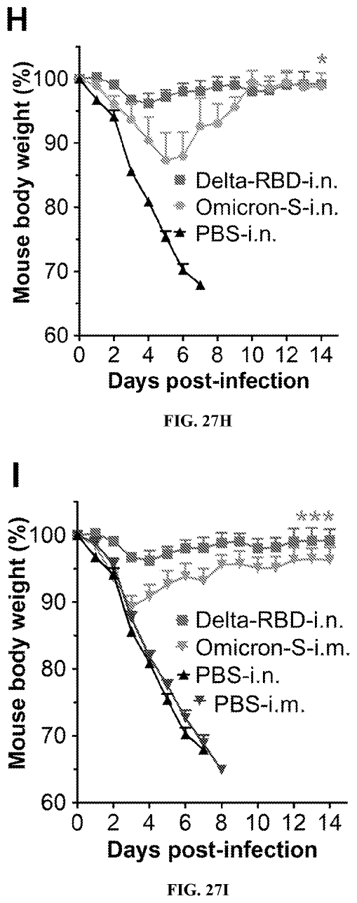

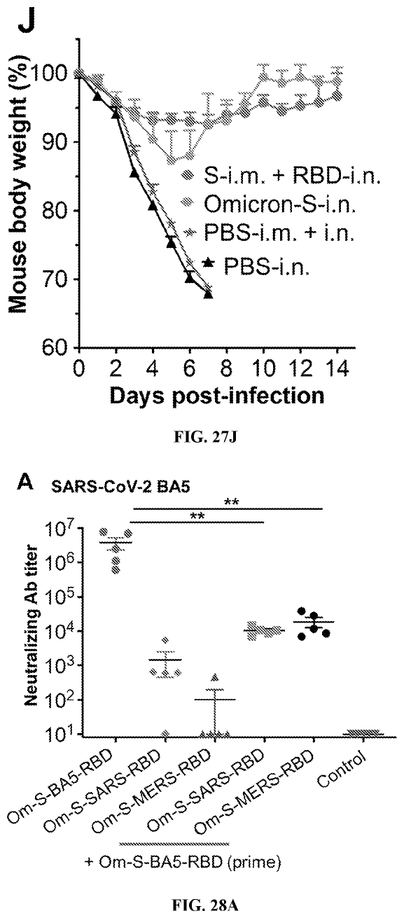

To evaluate the protective efficacy of the glycosylated Delta-RBD mucosal vaccine, immunized K18-hACE2 mice were challenged with the SARS-CoV-2 Delta variant 1 month after their last immunization. Mouse weight changes and overall survival were investigated for 14 days after challenge (FIG. 23A). The mice receiving Delta-RBD-i.n., with or without Omicron-S-i.m. priming, and those i.m. immunized with Delta-RBD or Omicron-S all survived the viral challenge (FIGS. 27A, 27B, 27C, and 27D); however, only 80% of the mice i.n. immunized with Omicron-S survived the challenge (FIG. 27E). Of note, mice receiving Delta-RBD via the i.m. route experienced slight weight loss in the first few days post-challenge but recovered all the weight loss during the experimental period; by contrast, the mice i.n. immunized with Delta-RBD did not experience obvious weight loss (FIG. 27F). The mice receiving Omicron-S protein via the i.m. route, and particularly those receiving Omicron-S via i.n. route, lost significantly more weight than the mice receiving Delta-RBD-i.n. (with or without Omicron-S-i.m. priming), especially on days 2 to 8 post-challenge (FIGS. 27G, 27H, 27I, and 27J). These data indicate that the i.n. delivery of glycosylated Delta-RBD (with or without Omicron-S-i.m. priming) protected immunized mice from Delta variant infection and prevented SARS-CoV-2-induced weight loss.

### Discussion

Currently, several COVID-19 vaccines have been approved for human use and many more have been developed preclinically. Although many of the first-generation vaccines induce strong systemic immune responses and protection against the original SARS-CoV-2 strain and early variants, their ability to protect against recent variants is markedly reduced. SARS-CoV-2 is a mucosal pathogen, which initiates infection in the upper respiratory tract mucosae, before reaching the lower respiratory tract and lungs. Thus, there is a need for mucosal vaccines to block viral transmission at the mucosal site. Accordingly, next-generation COVID-19 vaccines should be developed to prevent viral infection and transmission via the respiratory route and elicit broad-spectrum neutralizing antibodies targeting multiple SARS-CoV-2 variants.

Most of the currently developed mucosal COVID-19 vaccines are based on an adenovirus (Ad) vector delivery system. For instance, chimpanzee Ad (ChAd)-vectored trivalent mucosal COVID-19 vaccines expressing SARS-CoV-2 S1, nucleocapsid, and RNA-dependent RNA polymerase proteins induced local and systemic antibodies and protected mice against the original SARS-CoV-2 strain and the Alpha/B.1.1.7 and Beta/B.1.351 variants. Moreover, a human Ad5-vectored S-expressing vaccine delivered i.n. protected hamsters against severe SARS-CoV-2 infection and transmission. Meanwhile, the i.n. injection of a ChAd-vectored SARS-CoV-2-S vaccine elicited systemic and mucosal IgA antibodies, protecting the upper and lower respiratory tracts against SARS-CoV-2 infection. Finally, while a systemic S-mRNA vaccine alone elicited weak mucosal immune responses, its combination with a mucosal Ad-S vaccine resulted in the production of potent neutralizing antibodies against the original SARS-CoV-2 strain and the Omicron BA.1.1 subvariant.

It has been previously demonstrated that the systemic injection of a glycosylated SARS-CoV-2 wild-type RBD increased neutralizing antibody titers against the original RBD and its multiple variant forms, which offered more protection against SARS-CoV-2 infection than the wild-type RBD without glycosylation. Because the Delta variant induces higher levels of severe COVID-19 and death than the wild-type SARS-CoV-2 strain and the other known variants, it was the focus of the present example. Thus, herein a mucosal COVID-19 subunit vaccine was developed by fusing the glycosylated RBD of the Delta variant to the Fc of human IgG and evaluated its ability to induce mucosal immunity in the presence or absence of an Omicron-S protein. It was found that i.n. immunization with the glycosylated Delta-RBD vaccine alone elicited effective IgG and IgG subtype antibody responses, which were comparable to those induced by the i.m. immunization with this vaccine, but more balanced systemic antibodies (with lower IgG1/IgG2c ratio). Moreover, Delta-RBD-i.n. induced a more potent mucosal IgA antibody response in the BAL fluid of model mice, which completely protected these animals against lethal challenge with the Delta variant. Importantly, the Delta-RBD-i.n. mucosal vaccine alone induced an effective neutralizing antibody response against the original SARS-CoV-2 strain and several early variants (e.g., Delta and Omicron BA1), and its ability to elicit potent and broadly neutralizing antibodies against recent dominant variants (e.g., Omicron BA5 and XBB) was significantly increased by priming with Omicron-S.

To conclude, a mucosal COVID-19 vaccine was designed that elicited strong mucosal immune responses and broadly neutralizing antibody responses against multiple SARS-CoV-2 variants in mice, while offering complete protection against SARS-CoV-2-induced weight loss and death. In addition, an effective immunization strategy for improving the potency of broadly neutralizing antibodies was developed, which consisted of i.m. priming with an Omicron-S subunit vaccine followed by i.n. boosting with the Delta-RBD vaccine. These findings collectively imply that the Delta-RBD-i.n. mucosal vaccine can effectively prevent SARS-CoV-2 transmission at the respiratory mucosa, which is further improved with Omicron-S priming to protect against infection with known and emerging SARS-CoV-2 variants with pandemic potential.

### Example 6. A Pan-Coronavirus Subunit Vaccine Protects Against SARS-CoV-2 Omicron and MERS-CoV Challenge

In this example, a pan-coronavirus subunit vaccine was designed based on the conserved backbone of SARS-CoV-2 Omicron BA1 variant spike (S) protein (Om-S) (which contained HexaPro sequences and a C-terminal foldon trimeric sequence and His6 tag), by replacing its receptor-binding domain (RBD) with a MERS-CoV RBD (Om-S-MERS-RBD). Om-S-BA5-RBD and Om-S-SARS-RBD protein vaccines were constructed by replacing the above RBD with an Omicron BA5-RBD and a SARS-RBD, respectively.

### Materials and Methods

Mouse immunization and sample collection. Two separate immunizations were performed as described below. Female BALB/c mice (6-8-week-old) were randomly assigned to different groups, and intramuscularly (I.M.) immunized with the following proteins (10 μg/mouse) for 3 doses at a 3-week interval: 1) Om-S-BA5-RBD, for 3 doses; 2) Om-S-SARS-RBD, for 3 doses; 3) Om-S-MERS-RBD, for 3 doses; 4) Om-S-BA5-RBD for the 1^{st} dose and Om-S-SARS-RBD for the 2^{nd} and 3^{rd} doses; and 5) Om-S-BA5-RBD for the 1^{st} dose and Om-S-MERS-RBD for the 2^{nd} and 3^{d} doses. Aluminum (500 μg/mouse) and monophosphoryl lipid A (MPL, 10 μg/mouse) (InvivoGen) adjuvants were thoroughly mixed with the respective proteins before use. PBS plus the above adjuvants were included as control. Sera were collected 10 days after the last dose to test neutralizing antibodies against pseudotyped coronavirus infection as described below. The immunized mice were proceeded for the subsequent challenge studies.

Challenge of mice with SARS-CoV-2 Omicron-BA5 variant. The immunized mice described above were intranasally (I.N.) challenged with an Omicron-BA5 variant of SARS-CoV-2 at 10⁵ plaque-forming unit (PFU)/mouse (50 μl/mouse) 3 weeks after the last vaccination. The lungs of challenged mice were collected 2 days after challenge, and tested for viral titers and viral replication by plaque assay and qPCR method, respectively, as described below.

Challenge of mice with MERS-CoV. This was performed as described below. Three weeks after the last immunization, the vaccinated mice described above were I.N. transduced with 2.5×10⁸ focus-forming unit (FFU) of adenovirus 5-human DPP4 (Ad5CMV/hDPP4-myc-flag) suspended in 75 μl/DMEM culture medium, and then I.N. challenged with MERS-CoV (EMC2012 strain; 10⁵ PFU/mouse, 50 μl/mouse) 5 days after transduction. Lungs were collected three days after challenge, and measured for viral titers using plaque assay as described below.

Plaque assay. Lungs from mice challenged with SARS-CoV-2 Omicron-BA5 and MERS-CoV were homogenized in PBS. Tissue homogenate supernatants were serially diluted in DMEM cell culture medium. ACE2-TMPRSS2 (for SARS-CoV-2) or Vero 81 (for MERS-CoV) cells were plated in 12-well plates. and cultured at 37° C. for 1 h in 5% C02 with gentle rocking every 15 min. The medium was removed 1 h later, and the plates were overlaid with 0.6% agarose, which were removed after three days. The plaques were visualized by staining with 0.1% crystal violet. Viral titers were quantified as PFU/g of lung tissues.

qRT-PCR. Lungs from mice challenged with SARS-CoV-2 Omicron-BA5 variant were homogenized in Trizol buffer, and RNA was extracted according to the manufacturer's protocol (Invitrogen). Total RNA (1 μg) was used as a template for the first strand of cDNA, which was subjected to amplification of selected genes by real-time quantitative PCR (qRT-PCR) using Power SYBR Green PCR Master Mix (Applied Biosystems). The nucleocapsid (N) gene of Omicron-BA5 was detected using nCOV_N1 primer (IDT). The expression levels were normalized to glyceraldehyde-3-phosphate dehydrogenase (GAPDH) by the following threshold cycle (CT) equation: ΔCT=CT of the gene of interest−CT of GAPDH. All results are shown as a ratio to GAPDH calculated as 2−ΔCT.

Statistical analysis. Statistical software (GraphPad Prism 9) was used to determine statistical significance among different groups. Ordinary one-way ANOVA (Tukey's multiple comparison test) was applied for calculation. * **, and *** indicate P<0.05, P<0.01, and P<0.001, respectively.

### Results

Pan-Coronavirus Subunit Vaccines Induced Broadly Neutralizing Antibodies (nAbs) Against Multiple Coronaviruses

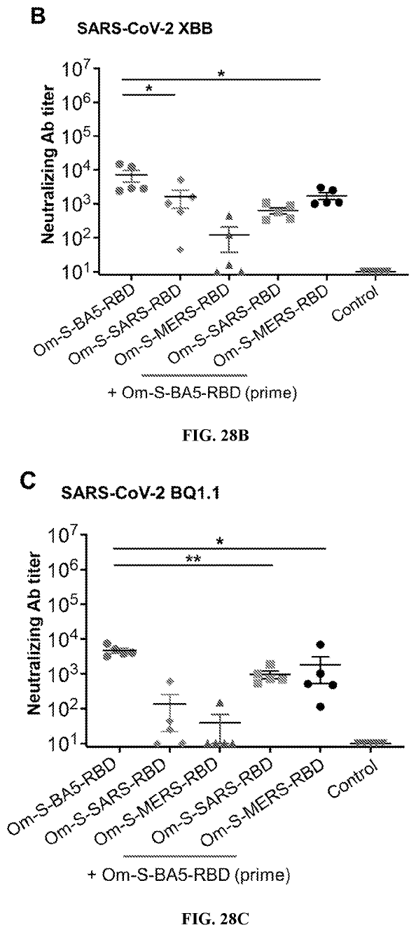

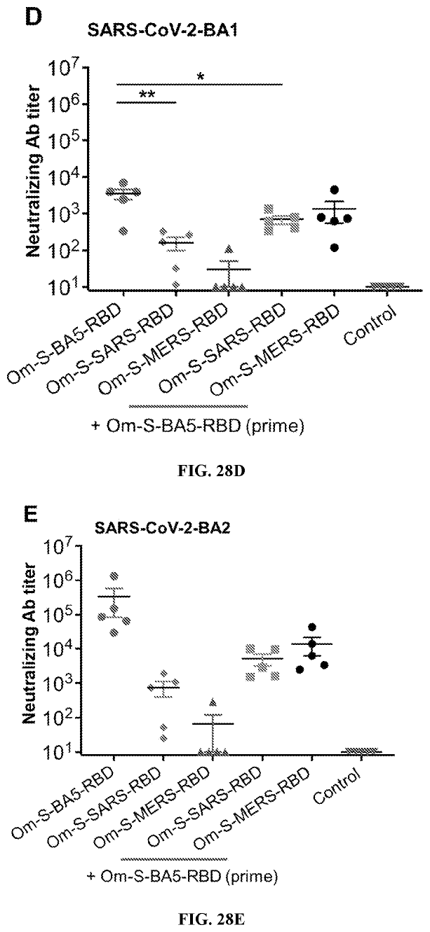

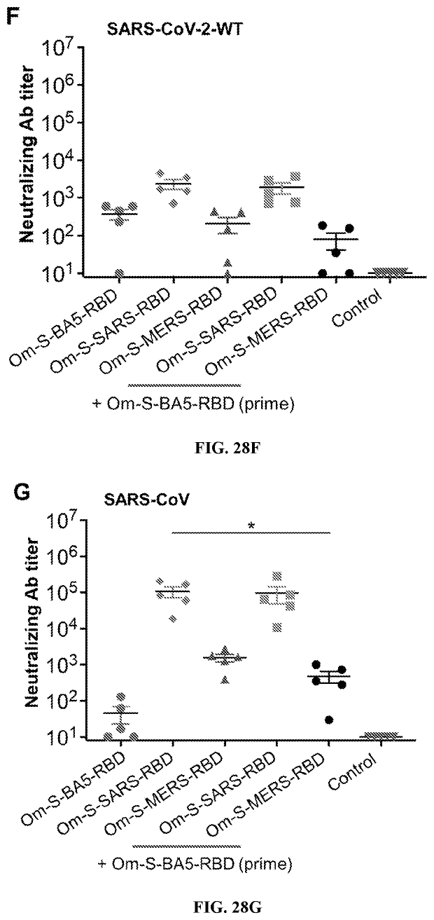

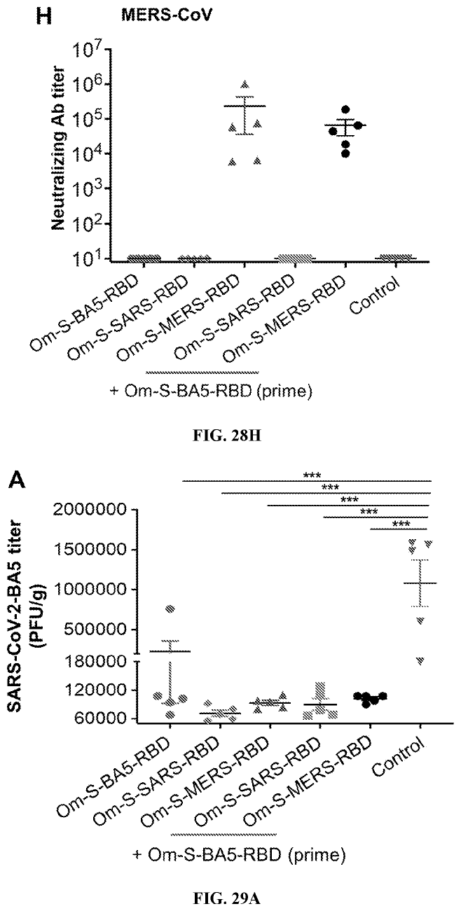

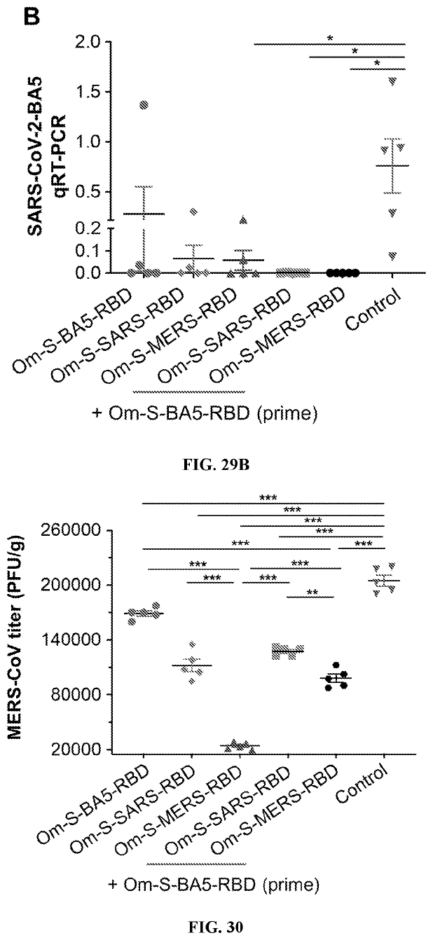

BALB/c mice were immunized with each of the aforementioned proteins, or a prime-boost strategy as described in the Materials and Methods section, and compared for the induced nAbs. Om-S-SARS-RBD protein alone induced high-titer anti-SARS-CoV nAbs and low-titer nAbs against the original SARS-CoV-2 wild-type (WT) strain and its variants (FIGS. 28A, 28B, 28C, 28D, 28E, 28F, and 28G). Om-S-MERS-RBD protein alone elicited high-titer anti-MERS-CoV nAbs and low-titer nAbs against the original strains of SARS-CoV-2 and SARS-CoV (FIGS. 28F, 28G, and 28H). Om-S-BA5-RBD protein alone induced high-titer nAbs against Omicron-BA2 and BA5 variants, relatively low-titer nAbs against the original SARS-CoV-2 and other variants, but no Abs against MERS-CoV (FIGS. 28A, 28B, 28C, 28D, 28E, 28F, 28G, and 28H). In contrast, Om-S-BA5-RBD protein priming followed by Om-S-SARS-RBD protein boosting elicited high-titer anti-SARS-CoV nAbs and broadly nAbs against the original SARS-CoV-2 and all variants tested, albeit with relatively lower nAb titers against some variants (FIGS. 28A, 28B, 28C, 28D, 28E, 28F, and 28G). Om-S-BA5-RBD protein priming followed by Om-S-MERS-RBD protein boosting elicited high-titer anti-MERS-CoV nAbs and broadly nAbs against all SARS-CoV-2 and SARS-CoV strains tested, with relatively low-titer nAbs against some of the variants (FIGS. 28A, 28B, 28C, 28D, 28E, 28F, 28G, and 28H). These data indicate that Om-S-MERS-RBD protein was developed as an effective universal vaccine against three highly pathogenic coronaviruses, including SARS-CoV-2, SARS-CoV, and MERS-CoV, and that priming with this vaccine followed by boosting with Om-S-BA5-RBD protein further improve its neutralizing activity against all virus strains tested.

Universal Om-S-MERS-RBD Subunit Vaccine Protected Immunized Mice Against Challenge with SARS-CoV-2 Omicron-BA5 Variant

To evaluate protective efficacy of the designed subunit vaccines against SARS-CoV-2 infection, immunized BALB/c mice were challenged with the SARS-CoV-2 (Omicron-BA5 variant) 3 weeks after the last vaccination, and measured for viral titers and viral replication in the lungs. All vaccination groups had significantly lower levels of viral titers and viral replication than the control mice receiving PBS plus adjuvants (FIG. 29). Compared with the other immunization groups, Om-S-MERS-RBD had relatively lower-level viral titers in the lungs of challenged mice, and the level of viral replication was also relatively lower (FIGS. 29A and 29B). Notably, mice priming with the Om-S-BA5-RBD followed by boosting with the Om-S-MERS-RBD further decreased viral replication (FIG. 29B). These data demonstrate that Om-S-MERS-RBD can be used as an effective vaccine to protect immunized mice against SARS-CoV-2 Omicron-BA5 infection and replication.

### Universal Om-S-MERS-RBD Subunit Vaccine Protected Immunized Mice Against MERS-CoV Challenge

To evaluate protective efficacy of the designed subunit vaccines against MERS-CoV infection, immunized BALB/c mice were challenged with an adenovirus-5-transduced MERS-CoV 3 weeks after the last dose, followed by measurement of viral titers in the lungs. Mice immunized with Om-S-MERS-RBD protein had low-levels of viral titers in the lungs, which were significantly lower than other vaccination groups, as well as the control group receiving the PBS plus adjuvants. Notably, mice immunized with the individual protein, or priming with the Om-S-BA5-RBD followed by boosting with either Om-S-SARS-RBD or Om-S-MERS-RBD also effectively blocked viral infection with relatively lower-titer virus in the lungs. These data reveal that designed universal subunit vaccines, particularly Om-S-MERS-RBD protein, effectively protect immunized mice against MERS-CoV infection.

### Example 7. a Universal mRNA Vaccine Elicits Protective Efficacy Against SARS-CoV-2 Omicron Variant and SARS-CoV

Highly pathogenic coronaviruses (CoVs) including SARS-CoV-2 and SARS-CoV have caused severe threats to global public health, leading to the COVID-19 pandemic and SARS outbreak, respectively. SARS-CoV-2 has undergone frequent mutations, particularly in the receptor-binding domain (RBD) of viral surface spike (S) protein, resulting in the inefficiency of previous COVID-19 vaccines against new variants, such as Omicron variant and its subvariants. Therefore, universal vaccines are urgently needed to prevent these CoVs and their variants. In this example, two new mRNA vaccines were designed by deleting the RBD of SARS-CoV-2 Omicron BA1 variant (SARS2-S-RBD-del) or replacing its RBD with the RBD of SARS-CoV (SARS2-S-SARS-RBD), and investigating for their immunogenicity and protective efficacy against SARS-CoV-2 and SARS-CoV infections. The data herein show that unlike SARS2-S-RBD-del mRNA, SARS2-S-SARS-RBD mRNA elicited potent antibodies specific to both SARS-CoV-2 S and SARS-CoV RBD proteins, capable of neutralizing infection of pseudotyped original SARS-CoV-2 and SARS-CoV strains. It also protected immunized mice from the challenge with the SARS-CoV-2 Omicron variant and SARS-CoV, with significantly reduced viral titers in the lungs after the Omicron challenge or completely preventing SARS-CoV-induced weight loss and death. Serum antibodies from SARS2-S-SARS-RBD mRNA-immunized mice protected naïve mice from SARS-CoV challenge with significantly reduced viral titers in the lungs, and the protective efficacy positively correlated with the serum neutralizing antibody titers.

SARS-CoV was first identified in 2002, and once led to the global outbreak with mortality rate of 10%. SARS-CoV-2 was first identified in 2019, and has caused the global Coronavirus Disease 2019 (COVID-19) pandemic, resulting in at least 767 million of human infections and more than 6.9 million of deaths as of May 31, 2023. SARS-CoV-2 has undergone frequent mutations, resulting in a variety of mutant variants, including current Omicron variant and its subvariants. Many previously developed COVID-19 vaccines have reduced ability against these new variants. Several SARS-like CoVs have been found in bats, and share the same receptor as SARS-CoV-2 and SARS-CoV. Therefore, universal vaccines are urgently needed to prevent infections of not only SARS-CoV-2 and its variants but also SARS-CoV or SARS-like CoVs.

Both SARS-CoV-2 and SARS-CoV belong to the Coronavirinae subfamily, Coronaviridae family, and the order of Nidovirales. The viral surface spike (S) proteins of these CoVs play a critical role in viral infection and pathogenesis by first binding to the cellular receptor angiotensin-converting enzyme 2 (ACE2) on the host cells through the receptor-binding domain (RBD) of S1 subunit. Therefore, both S protein and RBD fragment of SARS-CoV-2 and SARS-CoV are important vaccine targets. However, around 20 or more mutations have been identified in the RBD region of SARS-CoV-2 Omicron variant and its subvariants, significantly affecting the ability of vaccines covering the mutant RBD region or antibodies targeting the mutant RBD epitopes against various SARS-CoV-2 infections. Thus, a novel approach is needed to design universal vaccines against these coronaviruses, including different variants of SARS-CoV-2.

### Materials and Methods

Design and construct of mRNA vaccines. mRNAs used in this example were designed and constructed as described below. Specifically, recombinant SARS-CoV-2-S-RBD-del (SARS2-S-RBD-del) DNA was constructed containing the S gene (removing its RBD) of a SARS-CoV-2 Omicron BA1 with a N-terminal tPA signal peptide, a C-terminal foldon trimerization sequence, and a His6 tag. PCR was performed based on a codon-optimized S gene containing HexaPro sequences (i.e., mutated furin cleavage site and six proline substitutions) of SARS-CoV-2 Omicron BA1 variant. Recombinant SARS-CoV-2-S-SARS-CoV-RBD (SARS2-S-SARS-RBD) DNA was constructed by inserting the RBD gene sequence of SARS-CoV (Tor2 strain) into the above construct. The amplified PCR products were inserted into a pCAGGS-mCherry vector, and the recombinant plasmids were confirmed for correct sequences.

Synthesis of mRNA vaccines. mRNA vaccines were synthesized as described below. Specifically, after linearizing each of the aforementioned recombinant plasmid using BglII restriction enzyme. related mRNAs were synthesized using MEGAscript T7 Transcription Kit. Pseudo-UTP (Ψ) was added together with CTP, ATP and GTP nucleosides to form nucleotide-modified mRNAs in order to increase mRNA's stability. After purification, the mRNAs were capped with Cap 1 Capping System Kit, and tailed with Poly(A) Polymerase Tailing Kit according to the manufacturer's protocols.

mRNA formulation and characterization. To further increase mRNA's stability, above synthesized mRNAs were encapsulated with LNPs to form mRNA-LNPs for delivery. Specifically, each synthesized mRNA was diluted in PNI Formulation Buffer, and mixed with GenVoy-ILM (lipid mixture) at 3:1 ratio. This was performed using NanoAssemblr Ignite Instrument according to the manufacturer's protocols. The encapsulated mRNA-LNPs were concentrated using Amicon Ultra-15 Centrifugal Filters (10 kDa), and measured for endotoxin level using LAL Endotoxin Assay Kit (<1 EU/ml), as well as stability and particle size using DynaPro NanoStar II Light Scattering Detector.

Enzyme-linked immunoassay. Enzyme-linked immunoassay (ELISA) was performed to measure vaccine-induced serum antibodies. Specifically, 96-well ELISA plates were pre-coated with SARS-CoV-2 S-no RBD or SARS-CoV RBD protein (1 μg/ml) overnight at 4° C., and blocked with 2% non-fat milk in PBST buffer (PBS containing 0.05% Tween-20) for 1 h at 37° C. This was followed by addition of serially diluted mouse sera, and incubation for 1 h at 37° C. After three washes with PBST, the plates were incubated with horseradish peroxidase (HRP)-conjugated anti-mouse IgG-Fab (1:5,000), anti-mouse IgG1, or anti-mouse IgG2a (1:5,000) antibody for 1 h at 37° C. After further washes, the plates were incubated with substrate 3,3′,5,5′-Tetramethylbenzidine (TMB), followed by stopping of the reaction using H₂SO₄ (1 N), and measurement of absorbance at 450 nm using Cytation 7 Microplate Multi-Mode Reader and Gen5 software.

Pseudovirus preparation and pseudovirus neutralization assay. SARS-CoV-2 and SARS-CoV pseudoviruses were prepared as described below. Specifically, each of the recombinant plasmids encoding S protein of SARS-CoV-2 original strain or SARS-CoV (Tor2 strain) was co-transfected with pLenti-CMV-luciferase and PS-PAX2 plasmids into 293T cells using polyetherimide (PEI) transfection assay. After removing the medium, the transfected cells were incubated with fresh medium, and pseudovirus-containing supernatants were collected 72 h later, which were used for subsequent pseudovirus neutralization assay. Collected mouse sera were serially diluted, incubated with respective pseudovirus for 1 h at 37° C., and the virus-serum mixture was then added to hACE2/293T cells. Fresh medium was added to cells 24 h after infection, and the cells were continued to culture for 48 h. The cells were then lysed using cell lysis buffer, incubated with luciferase substrate, and measured for relative luciferase activity using Cytation 7 Microplate Multi-Mode Reader. Pseudovirus neutralizing activity of mouse serum antibodies was calculated as 50% neutralizing antibody titer (NT₅₀).

Plaque reduction neutralization assay. Mouse sera for passive transfer were assessed for neutralizing antibodies against live SARS-CoV infection using plaque reduction neutralization assay. Specifically, sera with serial dilutions were incubated with SARS-CoV (mouse-adapted MA15) for 1 h at 37° C., followed by addition of the virus-serum mixture to Vero E6, and incubation for 1 h at 37° C. After removal of the inoculations, the cells were overlayed with 0.6% agarose, and continued to culture for three days. After removal of the overlays, the plaques were stained with 0.1% crystal violet. 50% neutralizing antibody titer (NT₅₀) was reported as the highest serum dilution capable of reducing 50% of virus-induced plaques.

Plaque assay for detection of viral titers. Lungs from SARS-CoV-2 or SARS-CoV-challenged mice were detected for viral titers by a plaque assay. Specifically, lung tissues were homogenized in PBS, and collected for supernatant after centrifugation, which was followed by serial dilution of the supernatant in DMEM cell culture medium, and then incubation with Vero E6 cells for 1 h at 37° C. Other procedures were the same as described above. Viral titers in the lungs were calculated as PFU/g of lung tissues.

Mouse immunization procedures. BALB/c mice (6-8-week-old) were intradermally (I.D.) vaccinated with the SARS2-S-SARS-RBD mRNA-LNPs, SARS2-S-RBD-del mRNA-LNPs (10 μg/mouse), or LNP control, and boosted twice at 3 weeks. Sera collected 10 days after the last immunization were evaluated for specific IgG antibodies, IgG1 and Ig2a subtype antibodies, and neutralizing antibodies by ELISA and neutralization assays, respectively.

Challenge of immunized mice with SARS-CoV-2 Omicron variant. Forty days after the last immunization, mice were anesthetized, and then intranasally (I.N.) infected with SARS-CoV-2 Omicron BA1 variant (10⁵ plaque-forming unit (PFU)/mouse in 50 μl volume). Two days after infection, mice were euthanized, and lung tissues were collected for detection of viral titers by plaque assay as described above.

Challenge of immunized mice with SARS-CoV. Forty days after the last immunization, mice were anesthetized, I.N. infected with SARS-CoV (MA15 strain, 500 PFU/mouse in 50 μl volume), and then observed for survival and body weight loss for 14 days after infection.

Challenge of immune serum-transferred mice with SARS-CoV. Naïve mice were I.P. injected with pooled sera of mice collected 10 days after the last immunization of each mRNA vaccine or LNP control (200 μl/mouse). 12 h later, these mice were I.N. infected with SARS-CoV (MA15 strain, 400 PFU/mouse in 50 μl volume). Two days after infection, lungs were harvested from each mouse, and detected for viral titers by plaque assay as described above.

Statistical analysis. Statistical significance among different groups was analyzed using GraphPad Prism 9 statistical software. Statistical significance of lung viral titers and mouse weight changes were calculated using unpaired two-tailed student t test. *, **, and *** represent P<0.05, P<0.01, and P<0.001, respectively.

### Results

Construct and Synthesis of mRNA Vaccines

Two mRNA vaccines were constructed. SARS2-S-RBD-del mRNA was constructed to encode S protein (deleting the RBD) of SARS-CoV-2 BA1 variant with HexaPro sequences. SARS2-S-SARS-RBD mRNA was constructed by inserting the RBD of SARS-CoV into the above construct. Each mRNA cassette also contains a N-terminal tPA signal peptide, a C-terminal foldon trimeric sequence, and a C-terminal His6-tag. The constructed mRNAs were synthesized in the presence of nucleosides (ATP, GTP, and CTP) and a modified nucleoside (Pseudo-UTP) to increase stability and remove mRNA-associated innate immune activation. The synthesized mRNAs also contain a N-terminal 5′-untranslated region (5′-UTR) and a 3′-UTR, as well as a N-terminal Cap sequence and a C-terminal poly(A) tail. The synthesized mRNAs were formulated with lipid nanoparticle (LNPs) to form mRNA-LNPs to further improve their stability, and used as vaccines.

SARS2-S-SARS-RBD mRNA Vaccine Elicited Effective Antibodies with Neutralizing Activity Against SARS-CoV-2 and SARS-CoV Infections

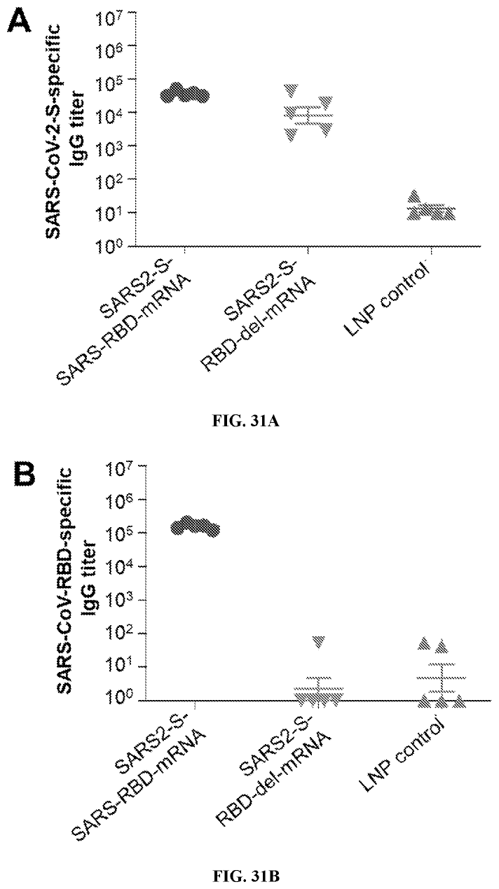

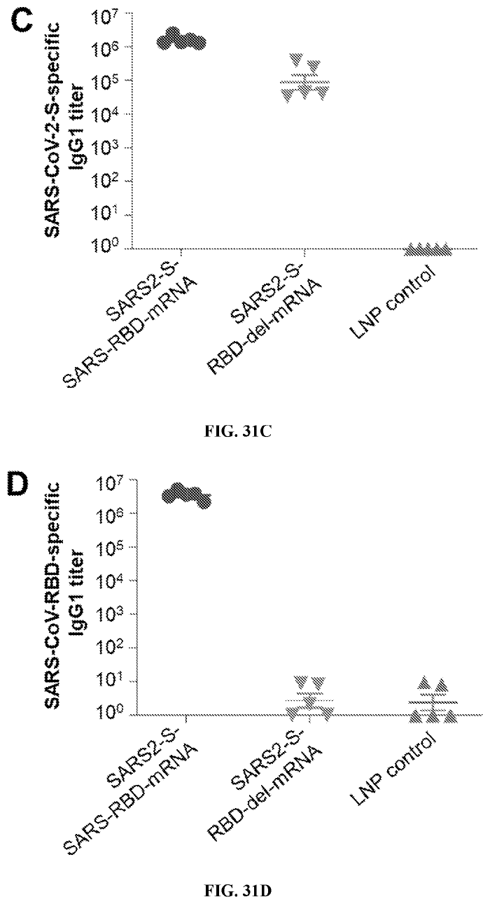

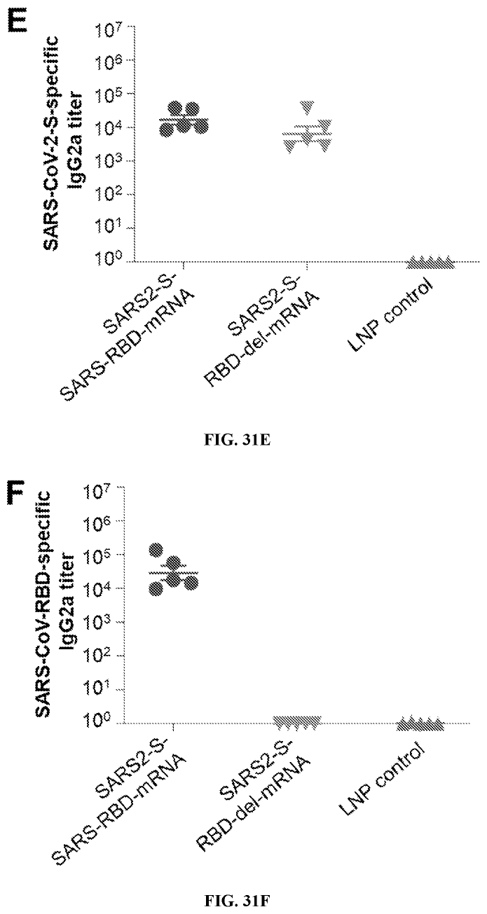

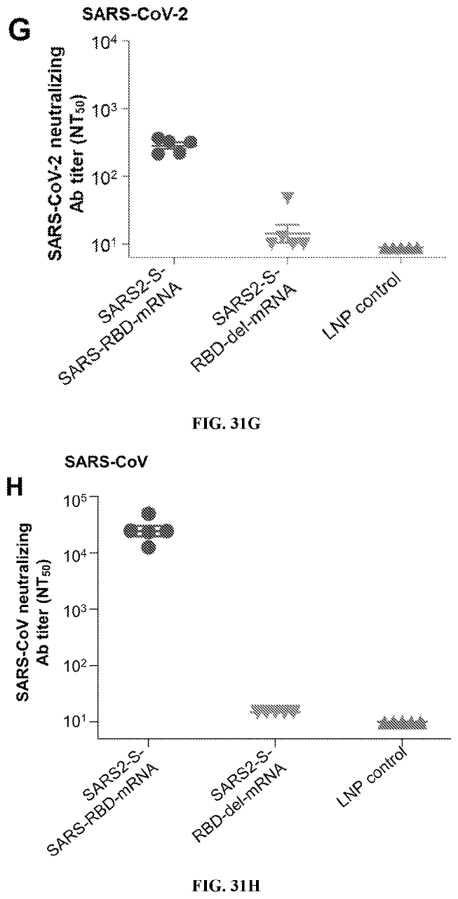

To investigate immunogenicity of mRNA vaccines to induce antibody responses, BALB/c mice were immunized with each LNP-formulated mRNA or LNP control, and sera were collected after the last immunization to test specific IgG antibodies, subtype (IgG1 and IgG2a) antibodies, and neutralizing antibodies. SARS2-S-SARS-RBD mRNA produced similar levels of IgG antibodies specific to both SARS-CoV-2 S and SARS-CoV RBD proteins, whereas SARS2-S-RBD-del mRNA-induced IgG antibodies only reacted with SASRS-CoV-2 S protein, but not against SARS-CoV RBD protein (FIGS. 31A and 31B). In addition, SARS2-S-SARS-RBD mRNA also elicited potent IgG1 and IgG2a subtype antibodies specific to SARS-CoV-2 S or SARS-CoV RBD protein, whereas SARS2-S-RBD-del mRNA-generated IgG1 and IgG2a antibodies only targeted SARS-CoV-2 S protein, rather than SARS-CoV RBD protein (FIGS. 31C, 31D, 31E, and 31F). Moreover, SARS2-S-SARS-RBD mRNA induced potent and high-titer neutralizing antibodies against infection of pseudotyped original SARS-CoV-2 and SARS-CoV strain tested, whereas SARS2-S-RBD-del mRNA induced low to no neutralizing antibodies against these pseudoviruses (FIGS. 31G and 31H). By contrast, LNP control failed to induce specific antibodies in neutralizing SARS-CoV-2 and SARS-CoV pseudovirus infections (FIG. 31). The above results illustrate that SARS2-S-SARS-RBD mRNA was capable of inducing strong antibodies with neutralizing activity against infection of both SARS-CoV-2 and SARS-CoV.

SARS2-S-SARS-RBD mRNA Vaccine Protected Immunized Mice Against Challenge with SARS-CoV-2 Omicron Variant and SARS-CoV

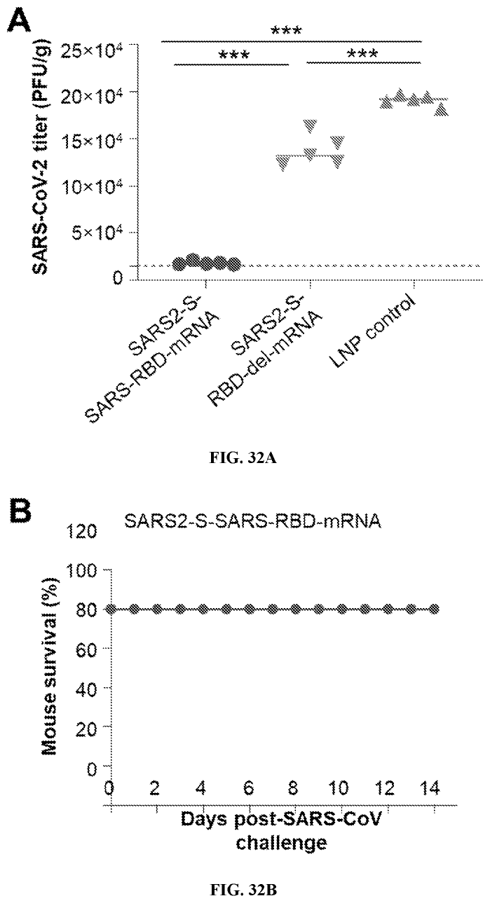

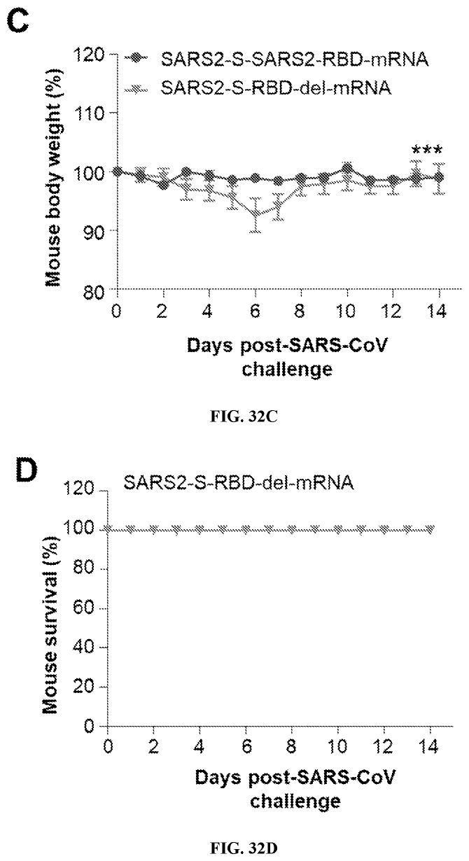

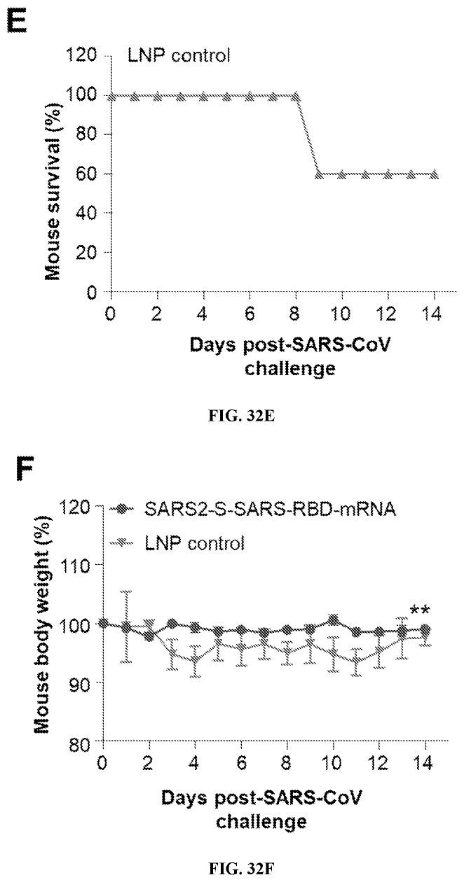

To evaluate protective efficacy of mRNA vaccines, BALB/c mice immunized with each mRNA or LNP control were separately challenged with the SARS-CoV-2 (Omicron BA1 variant) and SARS-CoV (MA15 strain) 40 days after the last dose of the vaccination, followed by measurement of viral titers, or observation of survival and weight changes, of challenged mice, respectively. SARS2-S-SARS-RBD mRNA potently inhibited SARS-CoV-2 infection, with significantly low-level viral titers in the lungs of challenged mice than those of mice immunized with the SARS2-S-RBD-del mRNA or LNP control; SARS2-S-RBD-del mRNA also inhibited SARS-CoV-2 infection to some extent, resulting in significantly lower viral titers in the lungs of immunized mice than those receiving LNP control (FIG. 32A). In addition, SARS2-S-SARS-RBD mRNA completely protected immunized mice against the SARS-CoV challenge, with 100% survival and without showing weight loss during the detection period of 14 days (FIGS. 32B and 32C). SARS2-S-RBD-del mRNA also fully protected immunized mice against the SARS-CoV challenge, but significant weight loss was observed in these mice, particularly during days 6-7 post-infection (FIGS. 32C and 32D). By contrast, 40% of mice injected with LNP control did not survive the SARS-CoV challenge, leading to significant weight loss, particularly during days 3-11 post-infection (FIGS. 32E and 32F). These data indicate that SARS2-S-SARS-RBD mRNA was able to induce protective efficacy in preventing both SARS-CoV-2 and SARS-CoV infections.

SARS2-S-SARS-RBD mRNA-Immune Sera Protected Naïve Mice Against SARS-CoV-2 Infection

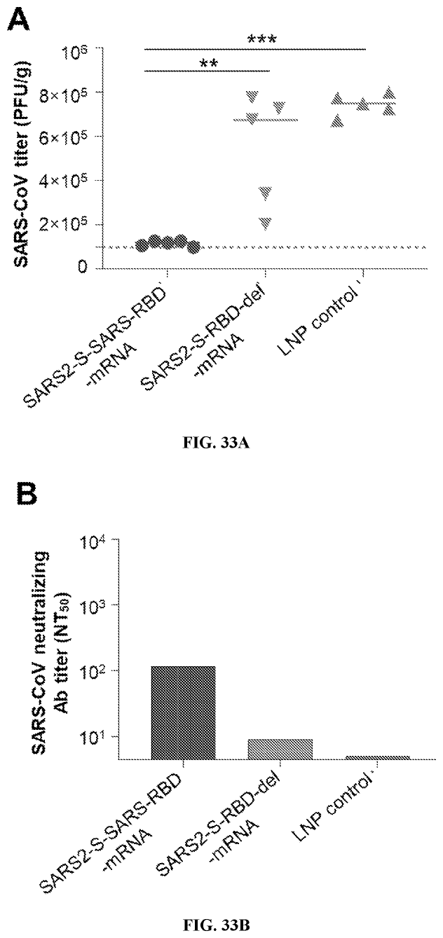

Since SARS2-S-SARS-RBD mRNA induced strong serum neutralizing antibodies against SARS-CoV infection in vitro, it was next evaluated whether these immunized serum antibodies were effective against SARS-CoV infection in vivo. As such, naïve BALB/c mice were passively transferred with each pooled serum from mice immunized with each mRNA or LNP control, challenged with the SARS-CoV (MA15 strain), and then detected for viral titers in the lungs two days later. Mice receiving immune sera of SARS2-S-SARS-RBD mRNA significantly reduced lung viral titers than mice receiving immune sera of SARS2-S-RBD-del mRNA and LNP control, respectively, whereas these SARS2-S-SARS-RBD mRNA-immunized mouse sera contained antibodies with effective neutralizing activity against authentic SARS-CoV (MA15) infection (FIGS. 33A and 33B). By contrast, the low-titer neutralizing antibodies in the sera of mice immunized with SARS2-S-RBD-del mRNA were not sufficient to protect against the SARS-CoV challenge, resulting in three out of five mice having high-level lung viral titers similar to those of mice receiving sera of LNP control (FIGS. 33A and 33B). These results reveal that immune serum antibodies of SARS2-S-SARS-RBD mRNA significantly prevented SARS-CoV infection in naïve mouse recipients, and the protection positively correlated with serum neutralizing antibody titers.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### SEQUENCES

1.
Amino acid sequences for generating original wild-type SARS-CoV-2
RBD-mRNA
SEQ ID NO: 1
NITNLCPFGEVFNATRFASVYAWNRKRISNCVADYSVLYNSASFSTFKCYGVSPTKLN
DLCFTNVYADSFVIRGDEVRQIAPGQTGKIADYNYKLPDDFTGCVIAWNSNNLDSKV
GGNYNYLYRLFRKSNLKPFERDISTEIYQAGSTPCNGVEGENCYFPLQSYGFQPTNGV
GYQPYRVVVLSFELLHAPATV
2.
Optimized DNA sequences of SARS-CoV-2 Omicron-BA.1 (with
HexaPro sequences) for generating BA1-S-mRNA vaccine
SEQ ID NO: 2
cagtgcgtgaatctgaccacaaggacccagctgccccctgcctacaccaactcct
tcacacggggcgtgtactatcccgacaaggtgtttagaagctccgtgctgcactc
tacacaggatctgtttctgcctttcttttctaatgtgacctggttccacgtgatc
agcggcaccaatggcaccaagaggttcgacaatcccgtgctgccttttaacgatg
gcgtgtacttcgccagcatcgagaagtccaacatcatccggggctggatctttgg
caccacactggactctaagacacagagcctgctgatcgtgaacaatgccaccaac
gtggtcatcaaggtgtgcgagttccagttttgtaacgatccattcctggaccaca
agaacaataagtcctggatggagtctgagtttagagtgtactctagcgccaacaa
ctgcacctttgagtatgtgagccagcccttcctgatggacctggagggcaagcag
ggcaatttcaagaacctgagagagttcgtgtttaagaatatcgatggctacttca
aaatctacagcaagcacacccccatcatcgtgagagagcctgaggatctgcccca
gggcttctccgccctggagccactggtggatctgcccatcggcatcaacatcacc
cggtttcagacactgctggccctgcacagatcctacctgacaccaggcgattcct
ctagcggctggaccgccggcgctgccgcctactatgtgggctatctgcagcccag
gaccttcctgclgaagtataacgagaalggcaccalcacagacgctgtggactgc
gccctggaccccctgagcgagaccaagtgtacactgaagtcctttaccgtggaga
agggcatctaccagacatccaatttcagggtgcagcctaccgagtctatcgtgcg
ctttccaaatatcacaaacctgtgcccctttgacgaggtgttcaacgccacccgc
ttcgccagcgtgtatgcctggaatcggaagcggatcagcaactgcgtggccgatt
atagcgtgctgtacaacctggcccctttctttacctttaagtgctatggcgtgag
ccccacaaagctgaatgacctgtgctttaccaacgtgtatgccgatagcttcgtg
atccggggcgacgaggtgagacagatcgccccaggccagacaggcaatatcgccg
actacaactataagctgcccgacgatttcaccggctgcgtgatcgcctggaactc
caataagctggattccaaggtgtctggcaactacaattatctgtacaggctgttt
cgcaagtctaatctgaagcctttcgagagagacatcagcacagaaatctaccagg
ccggaaataagccatgcaatggcgtggccggctttaactgttatttccctctgag
gagctactccttccgcccaacctatggcgtgggccaccagccctatagggtggtg
gtgctgtcctttgagctgctgcacgcccctgccacagtgtgcggccctaagaagt
ctaccaatctggtgaagaacaagtgcgtgaacttcaacttcaatggcctgaaggg
caccggcgtgctgacagagagcaacaagaagttcctgccttttcagcagttcggc
agagacatcgccgataccacagacgctgtgagagacccccagaccctggagatcc
tggacatcacaccatgctccttcggcggcgtgtctgtgatcacaccaggcaccaa
tacatccaaccaggtggctgtgctgtaccagggcgtgaattgtaccgaggtgccc
gtggcaatccacgcagatcagctgacccctacatggcgggtgtatagcaccggct
ccaacgtgttccagacaagagccggctgcctgatcggcgccgagtatgtgaacaa
ttcctacgagtgcgacatccccatcggcgccggcatctgtgcctcttaccagacc
cagaccaagagccacgggagcgccagcagcgtggcctctcagagcatcatcgcct
acaccatgagcctgggcgccgagaacagcgtggcctattccaacaattctatcgc
catccccaccaacttcacaatctctgtgaccacagagatcctgcctgtgagcatg
accaagacatccgtggactgcacaatgtacatctgtggcgatagcaccgagtgct
ccaatctgctgctgcagtatggctccttttgtacccagctgaagagggccctgac
aggcatcgctgtggagcaggataagaacacacaggaggtgttcgcccaggtgaag
caaatctacaagaccccacccatcaagtattttggcggcttcaacttcagccaga
tcctgccagaccccagcaagccttccaagcggagccccatcgaggatctgctgtt
caacaaggtgaccctggccgatgccggcttcatcaagcagtatggcgactgcctg
ggcgacatcgcagccagagacctgatctgtgcccagaagttcaagggcctgaccg
tgctgcctccactgctgacagatgagatgatcgcccagtatacaagcgccctgct
ggccggcaccatcacatccggctggacctttggcgccggccccgccctgcagatc
ccttttccaatgcagatggcctaccggttcaatggcatcggcgtgacccagaatg
tgctgtatgagaaccagaagctgatcgccaatcagttcaacagcgccatcggcaa
gatccaggacagcctgagcagcacccccagcgccctgggcaagctgcaggatgtg
gtgaatcacaacgcccaggccctgaataccctggtgaagcagctgtctagcaagt
tcggcgccatctcctctgtgctgaacgacatcttcagccggctggacccccccga
ggcagaggtgcagatcgatcggctgatcacaggcagactgcagtctctgcagacc
tacgtgacacagcagctgatcagggccgccgagatcagggccagcgccaatctgg
cagcaaccaagatgtccgagtgcgtgctgggccagtctaagagagtggatttttg
tggcaagggctaccacctgatgtctttcccacagagcgccccccacggggtggtg
tttctgcacgtgacctatgtgccagcccaggagaagaacttcaccacagcccccg
ccatctgccacgatggcaaggcacactttcctagggagggcgtgttcgtgagcaa
tggcacccactggtttgtgacacagcgcaacttctacgagccacagatcatcacc
acagacaatacattcgtgtccggcaactgtgacgtggtcatcggcatcgtgaaca
ataccgtgtacgatccactgcagcccgagctggacagctttaaggaggagctgga
taagtatttcaagaatcacacctcccccgacgtggatctgggcgacatctctggc
atcaatgccagcgtggtgaacatccagaaggagatcgacagactgaacgaggtgg
ccaagaatctgaacgagagcctgatcgatctgcaggagctgggcaagtatgagca
gtacatcaag
3.
N-terminal (PA signal peptide amino acid sequence
SEQ ID NO: 3
MDAMKRGLCCVLLLCGAVFVSAS
4.
C-terminal foldon and His6 tag (underlined) amino acid sequence
SEQ ID NO: 4
GYIPEAPRDGQAYVRKDGEWVLLSTFLGHHHHHH
5.
Amino acid sequences from translation of the above DNA sequences for
generating Omicron BA1-S mRNA (RBD is bolded).
The numbering was based on the S protein
of original SARS-CoV-2 strain (GenBank accession no.
QHR63250.2; residues H519 and P521
are underlined).
SEQ ID NO: 5
QCVNLTTRTQLPPAYTNSFTRGVYYPDKVFRSSVLHSTQDLFLPFFSNVTWFHVI
SGTNGTKRFDNPVLPFNDGVYFASIEKSNIIRGWIFGTTLDSKTQSLLIVNNATN
VVIKVCEFQFCNDPFLDHKNNKSWMESEFRVYSSANNCTFEYVSQPFLMDLEGKQ
GNFKNLREFVFKNIDGYFKIYSKHTPIIVREPEDLPQGFSALEPLVDLPIGINIT
RFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATR
FASVYAWNRKRISNCVADYSVLYNLAPFFTFKCYGVSPTKLNDLCFTNVYADSFV
IRGDEVRQIAPGQTGNIADYNYKLPDDFTGCVIAWNSNKLDSKVSGNYNYLYRLF
RKSNLKPFERDISTEIYQAGNKPCNGVAGENCYFPLRSYSFRPTYGVGHQPYRVV
VLSFELLHAPATVCGPKKSTNLVKNKCVNFNFNGLKGTGVLTESNKKFLPFQQFG
RDIADTTDAVRDPQTLEILDITPCSFGGVSVITPGTNTSNQVAVLYQGVNCTEVP
VAIHADQLTPTWRVYSTGSNVFQTRAGCLIGAEYVNNSYECDIPIGAGICASYQT
QTKSHGSASSVASQSIIAYTMSLGAENSVAYSNNSIAIPTNFTISVTTEILPVSM
TKTSVDCTMYICGDSTECSNLLLQYGSFCTQLKRALTGIAVEQDKNTQEVFAQVK
QIYKTPPIKYFGGFNFSQILPDPSKPSKRSPIEDLLFNKVTLADAGFIKQYGDCL
GDIAARDLICAQKFKGLTVLPPLLTDEMIAQYTSALLAGTITSGWTFGAGPALQI
PFPMQMAYRFNGIGVTQNVLYENQKLIANQFNSAIGKIQDSLSSTPSALGKLQDV
VNHNAQALNTLVKQLSSKFGAISSVLNDIFSRLDPPEAEVQIDRLITGRLQSLQT
YVTQQLIRAAEIRASANLAATKMSECVLGQSKRVDFCGKGYHLMSFPQSAPHGVV
FLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISG
INASVVNIQKEIDRLNEVAKNLNESLIDLQELGKYEQYIK
6.
Amino acid sequences of SARS-CoV-2 Omicron BA1-S protein
(containing N-terminal tPA signal peptide and C-terminal
foldon and His6 tag (underlined). RBD
is bolded):
SEQ ID NO: 6
MDAMKRGLCCVLLLCGAVFVSAS
QCVNLTTRTQLPPAYTNSFTRGVYYPDKVFRSSVLHSTQDLFLPFFSNVTWFHVISG
TNGTKRFDNPVLPFNDGVYFASIEKSNIIRGWIFGTTLDSKTQSLLIVNNATNVVIK
VCEFQFCNDPFLDHKNNKSWMESEFRVYSSANNCTFEYVSQPFLMDLEGKQGNFKNL
REFVFKNIDGYFKIYSKHTPIIVREPEDLPQGFSALEPLVDLPIGINITRFQTLLAL
HRSYLTPGDSSSGWTAGAAAYYVGYLQPRTFLLKYNENGTITDAVDCALDPLSETKC
TLKSFTVEKGIYQTSNFRVQPTESIVRFPNITNLCPFDEVFNATRFASVYAWNRKRI
SNCVADYSVLYNLAPFFTFKCYGVSPTKLNDLCFTNVYADSFVIRGDEVRQIAPGQT
GNIADYNYKLPDDFTGCVIAWNSNKLDSKVSGNYNYLYRLFRKSNLKPFERDISTEI
YQAGNKPCNGVAGFNCYFPLRSYSFRPTYGVGHQPYRVVVLSFELLHAPATVCGPKK
STNLVKNKCVNFNFNGLKGTGVLTESNKKFLPFQQFGRDIADTTDAVRDPQTLEILD
ITPCSFGGVSVITPGTNTSNQVAVLYQGVNCTEVPVAIHADQLTPTWRVYSTGSNVF
QTRAGCLIGAEYVNNSYECDIPIGAGICASYQTQTKSHgsasSVASQSIIAYTMSLG
AENSVAYSNNSIAIPTNFTISVTTEILPVSMTKTSVDCTMYICGDSTECSNLLLQYG
SFCTQLKRALTGIAVEQDKNTQEVFAQVKQIYKTPPIKYFGGFNFSQILPDPSKPSK
RSpIEDLLFNKVTLADAGFIKQYGDCLGDIAARDLICAQKFKGLTVLPPLLTDEMIA
QYTSALLAGTITSGWTFGAGpALQIPFpMQMAYRFNGIGVTQNVLYENQKLIANQFN
SAIGKIQDSLSSTpSALGKLQDVVNHNAQALNTLVKQLSSKFGAISSVLNDIFSRLDp
pEAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGTHW
FVTQRNFYEPQIITTDNTFVSGNCDVVIGIVNNTVYDPLQPELDSFKEELDKYFKNHT
SPDVDLGDISGINASVVNIQKEIDRLNEVAKNLNESLIDLQELGKYEQYIK
GYIPEAPRDGQAYVRKDGEWVLLSTFLGHHHHHH
7.
DNA sequences of SARS-CoV-2 MU-RBD DNA sequences:
SEQ ID NO: 7
aacatcaccaatctgtgccctttcggcgaggtgtttaacgccacacggttcgcct
ccgtgtatgcctggaaccggaagagaatctctaattgcgtggccgactatagcgt
gctgtacaatagcgcctccttctctacctttaagtgctatggcgtgtctccaacc
aagctgaacgacctgtgcttcacaaacgtgtacgccgacagctttgtgatccggg
gcgatgaggtgagacagatcgcccctggccagaccggcaagatcgcagactacaa
ctataagctgcctgacgacttcaccggctgcgtgatcgcctggaatagcaacaat
ctggattccaaagtgggcggcaactacaattatctgtacaggctgttccgcaagt
ccaacctgaagccatttgagagggacatctccaccgaaatctaccaggccggctc
tacaccctgcaacggcgtggagggcttcaattgttattttcccctgcagtcttac
ggcttccagcctaccaatggcgtgggctatcagccataccgcgtggtggtgctga
gctttgagctgctgaacgccaccgccacagtg
8.
Amino acid sequences (residues 331
524 of SARS-CoV-2 S protein)
(translated from the above MU-RBD DNA). The numbering was
based on the S protein of original SARS-CoV-2 strain
(GenBank accession no. QHR63250.2: glycosylation epitopes
519N and 521T are underlined.
SEQ ID NO: 8
NITNLCPFGEVENATRFASVYAWNRKRISNCVADYSVLYNSASFSTFKCYGVSPT
KLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGFNCYFPLQSY
GFQPTNGVGYQPYRVVVLSFELLNATATV
9.
Amino acid sequences of SARS-CoV-2 S-6P (containing N-terminal
tPA signal peptide and a C-terminal foldon and His6 tag (underlined).
RBD is bolded):
SEQ ID NO: 9
MDAMKRGLCCVLLLCGAVFVSASEF
QCVNLTTRTQLPPAYTNSFTRGVYYPDKVFRSSVLHSTQDLFLPFFSNVTWFHVI
SGTNGTKRFDNPVLPFNDGVYFASIEKSNIIRGWIFGTTLDSKTQSLLIVNNATN
VVIKVCEFQFCNDPFLDHKNNKSWMESEFRVYSSANNCTFEYVSQPFLMDLEGKQ
GNFKNLREFVFKNIDGYFKIYSKHTPIIVREPEDLPQGFSALEPLVDLPIGINIT
RFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATR
FASVYAWNRKRISNCVADYSVLYNLAPFFTFKCYGVSPTKLNDLCFTNVYADSFV
IRGDEVRQIAPGQTGNIADYNYKLPDDFTGCVIAWNSNKLDSKVSGNYNYLYRLF
RKSNLKPFERDISTEIYQAGNKPCNGVAGFNCYFPLRSYSFRPTYGVGHQPYRVV
VLSFELLHAPATVCGPKKSTNLVKNKCVNFNFNGLKGTGVLTESNKKFLPFQQFG
RDIADTTDAVRDPQTLEILDITPCSFGGVSVITPGTNTSNQVAVLYQGVNCTEVP
VAIHADQLTPTWRVYSTGSNVFQTRAGCLIGAEYVNNSYECDIPIGAGICASYQT
QTKSHGSASSVASQSIIAYTMSLGAENSVAYSNNSIAIPTNFTISVTTEILPVSM
TKTSVDCTMYICGDSTECSNLLLQYGSFCTQLKRALTGIAVEQDKNTQEVFAQVK
QIYKTPPIKYFGGFNFSQILPDPSKPSKRSPIEDLLFNKVTLADAGFIKQYGDCL
GDIAARDLICAQKFKGLTVLPPLLTDEMIAQYTSALLAGTITSGWTFGAGPALQI
PFPMQMAYRENGIGVTQNVLYENQKLIANQFNSAIGKIQDSLSSTPSALGKLQDV
VNHNAQALNTLVKQLSSKFGAISSVLNDIFSRLDPPEAEVQIDRLITGRLQSLQT
YVTQQLIRAAEIRASANLAATKMSECVLGQSKRVDFCGKGYHLMSFPQSAPHGVV
FLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISG
INASVVNIQKEIDRLNEVAKNLNESLIDLQELGKYEQYIK
GSGYIPEAPRDGQAYVRKDGEWVLLSTFLGHHHHHH
10.
Amino acid sequences of SARS-CoV-2 S-6P-Delta-RBD (containing N-
terminal tPA signal peptide and a C-terminal foldon and
His6 tag (underlined). SARS-CoV-2 Delta-RBD is bolded):
SEQ ID NO: 10
MDAMKRGLCCVLLLCGAVFVSASEF
QCVNLTTRTQLPPAYTNSFTRGVYYPDKVFRSSVLHSTQDLFLPFFSNVTWFHVI
SGTNGTKRFDNPVLPFNDGVYFASIEKSNIIRGWIFGTTLDSKTQSLLIVNNATN
VVIKVCEFQFCNDPFLDHKNNKSWMESEFRVYSSANNCTFEYVSQPFLMDLEGKQ
GNFKNLREFVFKNIDGYFKIYSKHTPIIVREPEDLPQGFSALEPLVDLPIGINIT
RFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFGEVFNATR
FASVYAWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNYRYRLF
RKSNLKPFERDISTEIYQAGSKPCNGVEGENCYFPLQSYGFQPTNGVGYQPYRVV
VLSFELLHAPATVCGPKKSTNLVKNKCVNFNFNGLKGTGVLTESNKKFLPFQQFG
RDIADTTDAVRDPQTLEILDITPCSFGGVSVITPGTNTSNQVAVLYQGVNCTEVP
VAIHADQLTPTWRVYSTGSNVFQTRAGCLIGAEYVNNSYECDIPIGAGICASYQT
QTKSHGSASSVASQSIIAYTMSLGAENSVAYSNNSIAIPTNFTISVTTEILPVSM
TKTSVDCTMYICGDSTECSNLLLQYGSFCTQLKRALTGIAVEQDKNTQEVFAQVK
QIYKTPPIKYFGGFNFSQILPDPSKPSKRSPIEDLLFNKVTLADAGFIKQYGDCL
GDIAARDLICAQKFKGLTVLPPLLTDEMIAQYTSALLAGTITSGWTFGAGPALQI
PFPMQMAYRENGIGVTQNVLYENQKLIANQFNSAIGKIQDSLSSTPSALGKLQDV
VNHNAQALNTLVKQLSSKFGAISSVLNDIFSRLDPPEAEVQIDRLITGRLQSLQT
YVTQQLIRAAEIRASANLAATKMSECVLGQSKRVDFCGKGYHLMSFPQSAPHGVV
FLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISG
INASVVNIQKEIDRLNEVAKNLNESLIDLQELGKYEQYIKGSGYIPEAPRDGQAY
VRKDGEWVLLSTFLGHHHHHH
11.
Amino acid sequences of SARS-CoV-2 S-6P-BA5-RBD (containing N-
terminal tPA signal peptide and a C-terminal foldon and His6
tag (underlined). SARS-CoV-2 Omicron BA5-RBD is bolded):
SEQ ID NO: 11
MDAMKRGLCCVLLLCGAVFVSASEFQCVNLTTRTQLPPAYTNSFTRGVYYPDKVF
RSSVLHSTQDLFLPFFSNVTWFHVISGINGTKRFDNPVLPFNDGVYFASIEKSNI
IRGWIFGTTLDSKTQSLLIVNNATNVVIKVCEFQFCNDPFLDHKNNKSWMESEFR
VYSSANNCTFEYVSQPFLMDLEGKQGNFKNLREFVFKNIDGYFKIYSKHTPIIVR
EPEDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQ
PTESIVRFPNITNLCTFDEVFNATRFASVYAWNRKRISNCVADYSVLYNFAPFFA
FKCYGVSPTKLNDLCFTNVYADSFVIRGNEVSQIAPGQTGNIADYNYKLPDDFTG
CVIAWNSNKLDSKVGGNYNYRYRLFRKSNLKPFERDISTEIYQAGNKPCNGVAGV
NCYFPLQSYGFRPTYGVGHQPYRVVVLSFELLHAPATVCGPKKSTNLVKNKCVNF
NFNGLKGTGVLTESNKKFLPFQQFGRDIADTTDAVRDPQTLEILDITPCSFGGVS
VITPGTNTSNQVAVLYQGVNCTEVPVAIHADQLTPTWRVYSTGSNVFQTRAGCLI
GAEYVNNSYECDIPIGAGICASYQTQTKSHGSASSVASQSIIAYTMSLGAENSVA
YSNNSIAIPTNFTISVTTEILPVSMTKTSVDCTMYICGDSTECSNLLLQYGSFCT
QLKRALTGIAVEQDKNTQEVFAQVKQIYKTPPIKYFGGFNFSQILPDPSKPSKRS
PIEDLLFNKVTLADAGFIKQYGDCLGDIAARDLICAQKFKGLTVLPPLLTDEMIA
QYTSALLAGTITSGWTFGAGPALQIPFPMQMAYRENGIGVTQNVLYENQKLIANQ
FNSAIGKIQDSLSSTPSALGKLQDVVNHNAQALNTLVKQLSSKFGAISSVLNDIF
SRLDPPEAEVQIDRLITGRLQSLQTYVTQQLIRAAEIRASANLAATKMSECVLGQ
SKRVDFCGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDPLQPELD
SFKEELDKYFKNHTSPDVDLGDISGINASVVNIQKEIDRLNEVAKNLNESLIDLQ
ELGKYEQYIKGSGYIPEAPRDGQAYVRKDGEWVLLSTFLGHHHHHH
12.
Amino acid sequences of SARS-CoV-2 S-6P-WT-RBD (containing N-
terminal tPA signal peptide and a C-terminal foldon and His6
tag (underlined). SARS-CoV-2 WT-
RBD is bolded):
SEQ ID NO: 12
MDAMKRGLCCVLLLCGAVFVSASEF
QCVNLTTRTQLPPAYTNSFTRGVYYPDKVFRSSVLHSTQDLFLPFFSNVTWFHVI
SGTNGTKRFDNPVLPFNDGVYFASIEKSNIIRGWIFGTTLDSKTQSLLIVNNATN
VVIKVCEFQFCNDPFLDHKNNKSWMESEFRVYSSANNCTFEYVSQPFLMDLEGKQ
GNFKNLREFVFKNIDGYFKIYSKHTPIIVREPEDLPQGFSALEPLVDLPIGINIT
RFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFGEVFNATR
FASVYAWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNYLYRLF
RKSNLKPFERDISTEIYQAGSTPCNGVEGENCYFPLQSYGFQPTNGVGYQPYRVV
VLSFELLHAPATVCGPKKSTNLVKNKCVNFNFNGLKGTGVLTESNKKFLPFQQFG
RDIADTTDAVRDPQTLEILDITPCSFGGVSVITPGTNTSNQVAVLYQGVNCTEVP
VAIHADQLTPTWRVYSTGSNVFQTRAGCLIGAEYVNNSYECDIPIGAGICASYQT
QTKSHGSASSVASQSIIAYTMSLGAENSVAYSNNSIAIPTNFTISVTTEILPVSM
TKTSVDCTMYICGDSTECSNLLLQYGSFCTQLKRALTGIAVEQDKNTQEVFAQVK
QIYKTPPIKYFGGFNFSQILPDPSKPSKRSPIEDLLFNKVTLADAGFIKQYGDCL
GDIAARDLICAQKFKGLTVLPPLLTDEMIAQYTSALLAGTITSGWTFGAGPALQI
PFPMQMAYRENGIGVTQNVLYENQKLIANQFNSAIGKIQDSLSSTPSALGKLQDV
VNHNAQALNTLVKQLSSKFGAISSVLNDIFSRLDPPEAEVQIDRLITGRLQSLQT
YVTQQLIRAAEIRASANLAATKMSECVLGQSKRVDFCGKGYHLMSFPQSAPHGVV
FLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISG
INASVVNIQKEIDRLNEVAKNLNESLIDLQELGKYEQYIKGSGYIPEAPRDGQAY
VRKDGEWVLLSTFLGHHHHHH
13.
Amino acid sequences of SARS-CoV-2 S-6P-no-RBD (containing N-
terminal tPA signal peptide and a C-terminal foldon and
His6 tag (underlined)):
SEQ ID NO: 13
MDAMKRGLCCVLLLCGAVFVSASEF
QCVNLTTRTQLPPAYTNSFTRGVYYPDKVFRSSVLHSTQDLFLPFFSNVTWFHVI
SGTNGTKRFDNPVLPFNDGVYFASIEKSNIIRGWIFGTTLDSKTQSLLIVNNATN
VVIKVCEFQFCNDPFLDHKNNKSWMESEFRVYSSANNCTFEYVSQPFLMDLEGKQ
GNFKNLREFVFKNIDGYFKIYSKHTPIIVREPEDLPQGFSALEPLVDLPIGINIT
RFQTLLALHRSYLTPGDSSSGWTAGAAAYYVGYLQPRTFLLKYNENGTITDAVDC
ALDPLSETKCTLKSFTVEKGIYQTSNFRVQPTESIVRFPCGPKKSTNLVKNKCVN
FNFNGLKGTGVLTESNKKFLPFQQFGRDIADTTDAVRDPQTLEILDITPCSFGGV
SVITPGTNTSNQVAVLYQGVNCTEVPVAIHADQLTPTWRVYSTGSNVFQTRAGCL
IGAEYVNNSYECDIPIGAGICASYQTQTKSHGSASSVASQSIIAYTMSLGAENSV
AYSNNSIAIPTNFTISVTTEILPVSMTKTSVDCTMYICGDSTECSNLLLQYGSFC
TQLKRALTGIAVEQDKNTQEVFAQVKQIYKTPPIKYFGGFNFSQILPDPSKPSKR
SPIEDLLFNKVTLADAGFIKQYGDCLGDIAARDLICAQKFKGLTVLPPLLTDEMI
AQYTSALLAGTITSGWTFGAGPALQIPFPMQMAYRFNGIGVTQNVLYENQKLIAN
QFNSAIGKIQDSLSSTPSALGKLQDVVNHNAQALNTLVKQLSSKFGAISSVLNDI
FSRLDPPEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDL
QELGKYEQYIKGSGYIPEAPRDGQAYVRKDGEWVLLSTFLGHHHHHH
14.
Amino acid sequences of SARS-CoV-2 S-6P-SARS-RBD (containing
N-terminal tPA signal peptide and a C-terminal foldon
and His6 tag (underlined). SARS-RBD is bolded):
SEQ ID NO: 14
MDAMKRGLCCVLLLCGAVFVSASEF
QCVNLTTRTQLPPAYTNSFTRGVYYPDKVFRSSVLHSTQDLFLPFFSNVTWFHVI
SGTNGTKRFDNPVLPFNDGVYFASIEKSNIIRGWIFGTTLDSKTQSLLIVNNATN
VVIKVCEFQFCNDPFLDHKNNKSWMESEFRVYSSANNCTFEYVSQPFLMDLEGKQ
GNFKNLREFVFKNIDGYFKIYSKHTPIIVREPEDLPQGFSALEPLVDLPIGINIT
RFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFGEVFNATK
FPSVYAWERKKISNCVADYSVLYNSTFFSTFKCYGVSATKLNDLCFSNVYADSFV
VKGDDVRQIAPGQTGVIADYNYKLPDDFMGCVLAWNTRNIDATSTGNYNYKYRYL
RHGKLRPFERDISNVPFSPDGKPCTPPALNCYWPLNDYGFYTTTGIGYQPYRVVV
LSFELLNAPATVCGPKKSTNLVKNKCVNFNFNGLKGTGVLTESNKKFLPFQQFGR
DIADTTDAVRDPQTLEILDITPCSFGGVSVITPGTNTSNQVAVLYQGVNCTEVPV
AIHADQLTPTWRVYSTGSNVFQTRAGCLIGAEYVNNSYECDIPIGAGICASYQTQ
TKSHGSASSVASQSIIAYTMSLGAENSVAYSNNSIAIPTNFTISVTTEILPVSMT
KTSVDCTMYICGDSTECSNLLLQYGSFCTQLKRALTGIAVEQDKNTQEVFAQVKQ
IYKTPPIKYFGGFNFSQILPDPSKPSKRSPIEDLLFNKVTLADAGFIKQYGDCLG
DIAARDLICAQKFKGLTVLPPLLTDEMIAQYTSALLAGTITSGWTFGAGPALQIP
FPMQMAYRFNGIGVTQNVLYENQKLIANQFNSAIGKIQDSLSSTPSALGKLQDVV
NHNAQALNTLVKQLSSKFGAISSVLNDIFSRLDPPEAEVQIDRLITGRLQSLQTY
VTQQLIRAAEIRASANLAATKMSECVLGQSKRVDFCGKGYHLMSFPQSAPHGVVF
LHVTYVPAQEKNFTTAPAICHDGKAHFPREGVFVSNGTHWFVTQRNFYEPQIITT
DNTFVSGNCDVVIGIVNNTVYDPLQPELDSFKEELDKYFKNHTSPDVDLGDISGI
NASVVNIQKEIDRLNEVAKNLNESLIDLQELGKYEQYIKGSGYIPEAPRDGQAYV
RKDGEWVLLSTFLGHHHHHH
15.
Amino acid sequence for SAR-CoV-2 S protein (Residues 519 and 521
are in bold, underline) (GenBank accession no. QHR63250.2)
SEQ ID NO: 15
MFVFLVLLPLVSSQCVNLTTRTQLPPAYTNSFTRGVYYPDKVFRSSVLHSTQDLF
LPFFSNVTWFHAIHVSGTNGTKRFDNPVLPFNDGVYFASTEKSNIIRGWIFGTTL
DSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGF
SALEPLVDLPIGINITRFQTLLALHRSYLTPGDSSSGWTAGAAAYYVGYLQPRTF
LLKYNENGTITDAVDCALDPLSETKCTLKSFTVEKGIYQTSNFRVQPTESIVRFP
NITNLCPFGEVFNATRFASVYAWNRKRISNCVADYSVLYNSASFSTFKCYGVSPT
KLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGFNCYFPLQSY
GFQPTNGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITPGTNTS
NQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIP
TNFTISVTTEILPVSMTKTSVDCTMYICGDSTECSNLLLQYGSFCTQLNRALTGI
AVEQDKNTQEVFAQVKQIYKTPPIKDFGGFNFSQILPDPSKPSKRSFIEDLLFNK
VTLADAGFIKQYGDCLGDIAARDLICAQKFNGLTVLPPLLTDEMIAQYTSALLAG
TITSGWTFGAGAALQIPFAMQMAYRFNGIGVTQNVLYENQKLIANQFNSAIGKIQ
DSLSSTASALGKLQDVVNQNAQALNTLVKQLSSNFGAISSVLNDILSRLDKVEAE
VQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVL
KGVKLHYT
16.
Amino acid sequence of SARS-CoV-2 RBD
SEQ ID NO: 16
NITNLCPFGEVFNATRFASVYAWNRKRISNCVADYSVLYNSASFSTFKCYGVSP
TKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPL
QSYGFQPTNGVGYQPYRVVVLSFELLHAPATV
17.
Amino acid sequences of SARS-CoV-2 S-6P-MERS-RBD (containing
N-terminal tPA signal peptide and a C-terminal foldon
and His6 tag (underlined). MERS-RBD is bolded)
SEQ ID NO: 17
MDAMKRGLCCVLLLCGAVFVSASEFQCVNLTTRTQLPPAYTNSFTRGVYYPDKVF
RSSVLHSTQDLFLPFFSNVTWFHVISGINGTKRFDNPVLPFNDGVYFASIEKSNI
IRGWIFGTTLDSKTQSLLIVNNATNVVIKVCEFQFCNDPFLDHKNNKSWMESEFR
VYSSANNCTFEYVSQPFLMDLEGKQGNFKNLREFVFKNIDGYFKIYSKHTPIIVR
EPEDLPQGFSALEPLVDLPIGINITRFQTLLALHRSYLTPGDSSSGWTAGAAAYY
VGYLQPRTFLLKYNENGTITDAVDCALDPLSETKCTLKSFTVEKGIYQTSNFRVQ
PTESIVRFPQAEGVECDFSPLLSGTPPQVYNFKRLVFTNCNYNLTKLLSLFSVND
FTCSQISPAAIASNCYSSLILDYFSYPLSMKSDLSVSSAGPISQFNYKQSFSNPT
CLILATVPHNLTTITKPLKYSYINKCSRLLSDDRTEVPQLVNANQYSPCVSIVPS
TVWEDGDYYRKQLSPLEGGGWLVASGSTVAMTEQLQMGFGITVQYGTDTNSVCPK
LCGPKKSTNLVKNKCVNFNFNGLKGTGVLTESNKKFLPFQQFGRDIADTTDAVRD
PQTLEILDITPCSFGGVSVITPGTNTSNQVAVLYQGVNCTEVPVAIHADQLTPTW
RVYSTGSNVFQTRAGCLIGAEYVNNSYECDIPIGAGICASYQTQTKSHGSASSVA
SQSIIAYTMSLGAENSVAYSNNSIAIPTNFTISVTTEILPVSMTKTSVDCTMYIC
GDSTECSNLLLQYGSFCTQLKRALTGIAVEQDKNTQEVFAQVKQIYKTPPIKYFG
GFNFSQILPDPSKPSKRSPIEDLLFNKVTLADAGFIKQYGDCLGDIAARDLICAQ
KFKGLTVLPPLLTDEMIAQYTSALLAGTITSGWTFGAGPALQIPFPMQMAYRFNG
IGVTQNVLYENQKLIANQFNSAIGKIQDSLSSTPSALGKLQDVVNHNAQALNTLV
KQLSSKFGAISSVLNDIFSRLDPPEAEVQIDRLITGRLQSLQTYVTQQLIRAAEI
RASANLAATKMSECVLGQSKRVDFCGKGYHLMSFPQSAPHGVVFLHVTYVPAQEK
NFTTAPAICHDGKAHFPREGVFVSNGTHWFVTQRNFYEPQIITTDNTFVSGNCDV
VIGIVNNTVYDPLQPELDSFKEELDKYFKNHTSPDVDLGDISGINASVVNIQKEI
DRLNEVAKNLNESLIDLQELGKYEQYIKGSGYIPEAPRDGQAYVRKDGEWVLLST
FLGHHHHHH
18.
Amino acid sequences of glycosylated SARS-CoV-2 Delta-RBD
(containing residues 331-524 of SARS-CoV-2 S protein with L452R and T478K mutations). The
numbering was based on the S protein of original SARS-CoV-2 strain (GenBank accession no.
QHR63250.2: mutant residues 452R and 478K, as well as glycosylation epitopes 519N and 521T
are underlined)
SEQ ID NO: 18
NITNLCPFGEVFNATRFASVYAWNRKRISNCVADYSVLYNSASFSTFKCYGVSPT
KLNDLCFTNVYADSFVIRGDEVRQIAPGQTGKIADYNYKLPDDFTGCVIAWNSNN
LDSKVGGNYNYRYRLFRKSNLKPFERDISTEIYQAGSKPCNGVEGENCYFPLQSY
GFQPTNGVGYQPYRVVVLSFELLNATATV
19.
Amino acid sequences of glycosylated SARS-CoV-2 Alpha (B.1.1.7) variant-
RBD (GISAID accession number EPI_ISL_17947761: containing residues 331-524 of SARS-
CoV-2 S protein with N501Y mutation). The numbering was based on the S protein of original
SARS-CoV-2 strain (GenBank accession no. QHR63250.2: mutant residue 501Y, as well as
glycosylation epitopes 519N and 521T, are underlined).
SEQ ID NO: 19
NITNLCPFGEVFNATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTYGVGYQP
YRVVVLSFELLNATATV
20.
Amino acid sequences of glycosylated SARS-CoV-2 Beta (B.1.351) variant-
RBD (GISAID accession number EPI ISL_17757975: containing residues 331-524 of SARS-
CoV-2 S protein with K417N, E484K, and N501Y mutations). The numbering was based on the S
protein of original SARS-CoV-2 strain (GenBank accession no. QHR63250.2: mutant residues
417N, 484K and 501Y, as well as glycosylation epitopes 519N and 521T, are underlined).
SEQ ID NO: 20
NITNLCPFGEVFNATRFASVYAWNRKRISNCVADYSVLYNSASFSTFKCYGVSPTKLND
LCFTNVYADSFVIRGDEVRQIAPGQTGNIADYNYKLPDDFTGCVIAWNSNNLDSKVGGN
YNYLYRLFRKSNLKPFERDISTEIYQAGSTPCNGVKGFNCYFPLQSYGFQPTYGVGYQP
YRVVVLSFELLNATATV
21.
Amino acid sequences of glycosylated SARS-CoV-2 Gamma (P.1) variant-
RBD (GISAID accession number EPI_ISL_17819680; containing residues 331-524 of SARS-
CoV-2 S protein with K417T, E484K, and N501Y mutations). The numbering was based on the S
protein of original SARS-CoV-2 strain (GenBank accession no. QHR63250.2: mutant residues
417T, 484K and 501Y, as well as glycosylation epitopes 519N and 521T, are underlined).
SEQ ID NO: 21
NITNLCPFGEVFNATRFASVYAWNRKRISNCVADYSVLYNSASFSTFKCYGVSPTKLND
LCFTNVYADSFVIRGDEVRQIAPGQTGTIADYNYKLPDDFTGCVIAWNSNNLDSKVGGN
YNYLYRLFRKSNLKPFERDISTEIYQAGSTPCNGVKGFNCYFPLQSYGFQPTYGVGYQP
YRVVVLSFELLNATATV
22.
Amino acid sequences of glycosylated SARS-CoV-2 Omicron-B.1.1.529
variant-RBD (GISAID accession number EPI ISL_6795835: containing residues 331-524 of
SARS-CoV-2 S protein with G339D, S371L, S373P, S375F, K417N, N440K, G446S, S477N,
T478K, E484A, Q493R, G496S, Q498R, N501Y, and Y505H mutations). The numbering was
based on the S protein of original SARS-CoV-2 strain (GenBank accession no. QHR63250.2:
mutant residues 339D, 371L, 373P, 375F, 417N, 440K, 446S, 477N, 478K, 484A, 493R, 496S,
498R, 501Y, and 505H, as well as glycosylation epitopes 519N and 521T, are underlined).
SEQ ID NO: 22
NITNLCPFDEVFNATRFASVYAWNRKRISNCVADYSVLYNLAPFFTFKCYGVSPTKLND
LCFTNVYADSFVIRGDEVRQIAPGQTGNIADYNYKLPDDFTGCVIAWNSNKLDSKVSGN
YNYLYRLFRKSNLKPFERDISTEIYQAGNKPCNGVAGFNCYFPLRSYSFRPTYGVGHQP
YRVVVLSFELLNATATV
23.
Amino acid sequences of glycosylated SARS-CoV-2 Omicron-
XBB.1.9.1 variant-RBD (GISAID accession number EPI_ISL_17950772: containing residues
331-524 of SARS-CoV-2 S protein mutations G339H, R346T, L368I, S371F, S373P, S375F,
T376A, D405N, R408S, K417N, N440K, V445P, G446S, N460K, S477N, T478K, E484A, F486P,
F490S, Q498R, N501Y, and Y505H mutations). The numbering was based on the S protein of
original SARS-CoV-2 strain (GenBank accession no. QHR63250.2: mutant residues 339H, 346T,
368I, 371F, 373P, 375F, 376A, 405N, 408S, 417N, 440K, 445P, 446S, 460K, 477N, 478K, 484A,
486P, 490S, 498R, 501Y, and 505H, as well as glycosylation epitopes 519N and 521T, are
underlined).
SEQ ID NO: 23
NITNLCPFHEVFNATTFASVYAWNRKRISNCVADYSVIYNFAPFFAFKCYGVSPTKLNDL
CFTNVYADSFVIRGNEVSQIAPGQTGNIADYNYKLPDDFTGCVIAWNSNKLDSKPSGNY
NYLYRLFRKSKLKPFERDISTEIYQAGNKPCNGVAGPNCYSPLQSYGFRPTYGVGHQPY
RVVVLSFELLNATATV
24.
Amino acid sequences of glycosylated SARS-CoV-2 Omicron-
XBB.1.9.2 variant-RBD (GISAID accession number EPI_ISL_17950599: containing residues
331-524 of SARS-CoV-2 S protein mutations G339H, R346T, L368I, S371F, S373P, S375F,
T376A, D405N, R408S, K417N, N440K, V445P, G446S, F456L, N460K, S477N, T478K,
E484A, F486P, F490S, Q498R, N501Y, and Y505H mutations). The numbering was based on the
S protein of original SARS-CoV-2 strain (GenBank accession no. QHR63250.2: mutant residues
339H, 346T, 3681, 371F, 373P, 375F, 376A, 405N, 408S, 417N, 440K, 445P, 446S, 456L, 460K,
477N, 478K, 484A, 486P, 490S, 498R, 501Y, and 505H, as well as glycosylation epitopes 519N
and 521T, are underlined).
SEQ ID NO: 24
NITNLCPFHEVFNATTFASVYAWNRKRISNCVADYSVIYNFAPFFAFKCYGVSPTKLNDL
CFTNVYADSFVIRGNEVSQIAPGQTGNIADYNYKLPDDFTGCVIAWNSNKLDSKPSGNY
NYLYRLLRKSKLKPFERDISTEIYQAGNKPCNGVAGPNCYSPLQSYGFRPTYGVGHQPY
RVVVLSFELLNATATV
25.
Amino acid sequences of glycosylated SARS-CoV-2 Omicron-BA.2-RBD
(GISAID accession number EPI_ISL_12030355.2: containing residues 331-524 of SARS-CoV-2
S protein with G339D, S371F, S373P, S375F, T376A, D405N, R408S, K417N, N440K, E S477N,
T478K, 484A, Q493R, Q498R, N501Y, and Y505H mutations). The numbering was based on the
S protein of original SARS-CoV-2 strain (GenBank accession no. QHR63250.2: mutant residues
339D, 371F, 373P, 375F, 376A, 405N, 408S, 417N, 440K, 477N, 478K, 484A, 493R, 498R, 501Y,
and 505H, as well as glycosylation epitopes 519N and 521T, are underlined).
SEQ ID NO: 25
NITNLCPFDEVFNATRFASVYAWNRKRISNCVADYSVLYNFAPFFAFKCYGVSPTKLND
LCFTNVYADSFVIRGNEVSQIAPGQTGNIADYNYKLPDDFTGCVIAWNSNKLDSKVGGN
YNYLYRLFRKSNLKPFERDISTEIYQAGNKPCNGVAGENCYFPLRSYGFRPTYGVGHQP
YRVVVLSFELLNATATV
26.
Amino acid sequences of glycosylated SARS-CoV-2 Omicron-BA.2.75-
RBD (GISAID accession number EPI ISL 17830049: containing residues 331-524 of SARS-
CoV-2 S protein with G339H, S371F, S373P, S375F, T376A, D405N, R408S, K417N, N440K,
G446S, N460K, S477N, T478K, E484A, F486L, Q498R, N501Y, and Y505H mutations). The
numbering was based on the S protein of original SARS-CoV-2 strain (GenBank accession no.
QHR63250.2: mutant residues 339H, 371F, 373P, 375F, 376A, 405N, 408S, 417N, 440K, 446S,
460K, 477N, 478K, 484A, 486L, 498R, 501Y, and 505H, as well as glycosylation epitopes 519N
and 521T, are underlined).
SEQ ID NO: 26
NITNLCPFHEVFNATRFASVYAWNRKRISNCVADYSVLYNFAPFFAFKCYGVSPTKLND
LCFTNVYADSFVIRGNEVSQIAPGQTGNIADYNYKLPDDFTGCVIAWNSNKLDSKVSGN
YNYLYRLFRKSKLKPFERDISTEIYQAGNKPCNGVAGLNCYFPLQSYGFRPTYGVGHQP
YRVVVLSFELLNATATV
27.
Amino acid sequences of glycosylated SARS-CoV-2 Omicron-
BA.2.75.2-RBD (GISAID accession number EPI_ISL_15213736: containing residues 331-524 of
SARS-CoV-2 S protein G339H, R346T, S371F, S373P, S375F, T376A, D405N, R408S, K417N,
N440K, G446S, N460K, S477N, T478K, E484A, F486S, Q498R, N501Y, and Y505H mutations).
The numbering was based on the S protein of original SARS-CoV-2 strain (GenBank accession
no. QHR63250.2: mutant residues 339H, 346T, 371F, 373P, 375F, 376A, 405N, 408S, 417N,
440K, 446S, 460K, 477N, 478K, 484A, 486S, 498R, 501Y, and 505H, as well as glycosylation
epitopes 519N and 521T, are underlined).
SEQ ID NO: 27
NITNLCPFHEVFNATTFASVYAWNRKRISNCVADYSVLYNFAPFFAFKCYGVSPTKLND
LCFTNVYADSFVIRGNEVSQIAPGQTGNIADYNYKLPDDFTGCVIAWNSNKLDSKVSGN
YNYLYRLFRKSKLKPFERDISTEIYQAGNKPCNGVAGSNCYFPLQSYGFRPTYGVGHQP
YRVVVLSFELLNATATV
28.
Amino acid sequences of glycosylated SARS-CoV-2 Omicron-
BA.2.75.3-RBD (GISAID accession number EPI_ISL_14384334: containing residues 331-524 of
SARS-CoV-2 S protein G339H, S371F, S373P, S375F, T376A, D405N, R408S, K417N, N440K,
G446S, N460K, S477N, T478K, E484A, Q498R, N501Y, and Y505H mutations). The numbering
was based on the S protein of original SARS-CoV-2 strain (GenBank accession no. QHR63250.2:
mutant residues 339H, 371F, 373P, 375F, 376A, 405N, 408S, 417N, 440K, 446S, 460K, 477N,
478K, 484A, 498R, 501Y, and 505H, as well as glycosylation epitopes 519N and 521T, are
underlined).
SEQ ID NO: 28
NITNLCPFHEVFNATRFASVYAWNRKRISNCVADYSVLYNFAPFFAFKCYGVSPTKLND
LCFTNVYADSFVIRGNEVSQIAPGQTGNIADYNYKLPDDFTGCVIAWNSNKLDSKVSGN
YNYLYRLFRKSKLKPFERDISTEIYQAGNKPCNGVAGFNCYFPLQSYGFRPTYGVGHQP
YRVVVLSFELLNATATV
29.
Amino acid sequences of glycosylated SARS-CoV-2 CH.1.1 variant-
RBD (GISAID accession number EPI_ISL_17885485: containing residues 331-524 of SARS-
CoV-2 S protein G339H, R346T, S371F, S373P, S375F, T376A, D405N, R408S, K417N, N440K,
K444T, G446S, L452R, N460K, S477N, T478K, E484A, F486S, Q498R, N501Y, and Y505H
mutations). The numbering was based on the S protein of original SARS-CoV-2 strain (GenBank
accession no. QHR63250.2: mutant residues 339H, 346T, 371F, 373P, 375F, 376A, 405N, 408S,
417N, 440K, 444T, 446S, 452R, 460K, 477N, 478K, 484A, 486S, 498R, 501Y, and 505H, as well
as glycosylation epitopes 519N and 521T, are underlined).
SEQ ID NO: 29
NITNLCPFHEVFNATTFASVYAWNRKRISNCVADYSVLYNFAPFFAFKCYGVSPTKLND
LCFTNVYADSFVIRGNEVSQIAPGQTGNIADYNYKLPDDFTGCVIAWNSNKLDSTVSGN
YNYRYRLFRKSKLKPFERDISTEIYQAGNKPCNGVAGSNCYFPLQSYGFRPTYGVGHQP
YRVVVLSFELLNATATV
30.
Amino acid sequences of glycosylated SARS-CoV-2 Omicron-XBB.2.3
variant-RBD (GISAID accession number EPI_ISL_17950790: containing residues 331-524 of
SARS-CoV-2 S protein mutations G339H, R346T, L368I, S371F, S373P, S375F, T376A, D405N,
R408S, K417N, N440K, V445P, G446S, N460K, S477N, T478K, E484A, F486P, F490S, S494P,
Q498R, N501Y, and Y505H mutations). The numbering was based on the S protein of original
SARS-CoV-2 strain (GenBank accession no. QHR63250.2: mutant residues 339H, 346T, 368I,
371F, 373P, 375F, 376A, 405N, 408S, 417N, 440K, 445P, 446S, 460K, 477N, 478K, 484A, 486P,
490S, 494P, 498R, 501Y, and 505H, as well as glycosylation epitopes 519N and 521T, are
underlined).
SEQ ID NO: 30
NITNLCPFHEVFNATTFASVYAWNRKRISNCVADYSVIYNFAPFFAFKCYGVSPTKLNDL
CFTNVYADSFVIRGNEVSQIAPGQTGNIADYNYKLPDDFTGCVIAWNSNKLDSKPSGNY
NYLYRLFRKSKLKPFERDISTEIYQAGNKPCNGVAGPNCYSPLQPYGFRPTYGVGHQPY
RVVVLSFELLNATATV
31.
Amino acid sequences of glycosylated SARS-CoV-2 Omicron-BA.4.6
variant-RBD (GISAID accession number EPI_ISL_14288784: containing residues 331-524 of
SARS-CoV-2 S protein G339D, R346T, S371F, S373P, S375F, T376A, D405N, R408S, K417N,
N440K, L452R, S477N, T478K, E484A, F486V, Q498R, N501Y, and Y505H mutations). The
numbering was based on the S protein of original SARS-CoV-2 strain (GenBank accession no.
QHR63250.2: mutant residues 339D, 346T, 371F, 373P, 375F, 376A, 405N, 408S, 417N, 440K,
452R, 477N, 478K, 484A, 486V, 498R, 501Y, and 505H, as well as glycosylation epitopes 519N
and 521T, are underlined).
SEQ ID NO: 31
NITNLCPFDEVFNATTFASVYAWNRKRISNCVADYSVLYNFAPFFAFKCYGVSPTKLND
LCFTNVYADSFVIRGNEVSQIAPGQTGNIADYNYKLPDDFTGCVIAWNSNKLDSKVGGN
YNYRYRLFRKSNLKPFERDISTEIYQAGNKPCNGVAGVNCYFPLQSYGFRPTYGVGHQP
YRVVVLSFELLNATATV
32.
Amino acid sequences of glycosylated SARS-CoV-2 Omicron-BA5 variant-
RBD (GISAID accession number EPI_ISL_12043290: containing residues 331-524 of SARS-
CoV-2 S protein with G339D, S371F, S373P, S375F, T376A, D405N, R408S, K417N, N440K,
L452R, S477N, T478K, E484A, F486V, Q498R, N501Y, and Y505H mutations). The numbering
was based on the S protein of original SARS-CoV-2 strain (GenBank accession no. QHR63250.2:
mutant residues, 339D, 371F, 373P, 375F, 376A, 405N, 408S, 417N, 440K, 452R, 477N, 478K,
484A, 486V, 498R, 501Y, 505H, as well as glycosylation epitopes 519N and 521T, are
underlined).
SEQ ID NO: 32
NITNLCPFDEVFNATRFASVYAWNRKRISNCVADYSVLYNFAPFFAFKCYGVSPTKLND
LCFTNVYADSFVIRGNEVSQIAPGQTGNIADYNYKLPDDFTGCVIAWNSNKLDSKVGGN
YNYRYRLFRKSNLKPFERDISTEIYQAGNKPCNGVAGVNCYFPLQSYGFRPTYGVGHQP
YRVVVLSFELLNATATV
33.
Amino acid sequences of glycosylated SARS-CoV-2 Omicron-XBB1.5
variant-RBD (GISAID accession number EPI_ISL_17719128: containing residues 331-524 of
SARS-CoV-2 S protein with G339H, R346T, L368I, S371F, S373P, S375F, T376A, D405N,
R408S, K417N, N440K, V445P, G446S, N460K, S477N, T478K, E484A, F486P, F490S, Q498R,
N501Y, and Y505H mutations). The numbering was based on the S protein of original SARS-
CoV-2 strain (GenBank accession no. QHR63250.2: mutant residues, 339H, 346T, 3681, 376A,
371F, 373P, 375F, 405N, 408S, 417N, 440K, 445P, 446S, 460K, 477N, 478K, 484A, 486P, 490S,
498R, 501Y, 505H, as well as glycosylation epitopes 519N and 521T, are underlined).
SEQ ID NO: 33
NITNLCPFHEVFNATTFASVYAWNRKRISNCVADYSVIYNFAPFFAFKCYGVSPTKLNDL
CFTNVYADSFVIRGNEVSQIAPGQTGNIADYNYKLPDDFTGCVIAWNSNKLDSKPSGNY
NYLYRLFRKSKLKPFERDISTEIYQAGNKPCNGVAGPNCYSPLQSYGFRPTYGVGHQPY
RVVVLSFELLNATATV
34.
Amino acid sequences of glycosylated SARS-CoV-2 Omicron-XBB1.16
variant-RBD (GISAID accession number EPI_ISL_17609306: containing residues 331-524 of
SARS-CoV-2 S protein with G339H, R346T, L368I, S371F, S373P, S375F, T376A, D405N,
R408S, K417N, N440K, V445P, G446S, N460K, S477N, T478R, E484A, F486P, F490S, Q498R,
N501Y, and Y505H mutations). The numbering was based on the S protein of original SARS-
CoV-2 strain (GenBank accession no. QHR63250.2: mutant residues, 339H, 346T, 3681, 371F,
373P, 375F, 376A, 405N, 408S, 417N, 440K, 445P, 446S, 460K, 477N, 478R, 484A, 486P, 490S,
498R, 501Y, and 505H, as well as glycosylation epitopes 519N and 521T, are underlined).
SEQ ID NO: 34
NITNLCPFHEVFNATTFASVYAWNRKRISNCVADYSVIYNFAPFFAFKCYGVSPTKLNDL
CFTNVYADSFVIRGNEVSQIAPGQTGNIADYNYKLPDDFTGCVIAWNSNKLDSKPSGNY
NYLYRLFRKSKLKPFERDISTEIYQAGNRPCNGVAGPNCYSPLQSYGFRPTYGVGHQPYR
VVVLSFELLNATATV
35.
Amino acid sequences of glycosylated SARS-CoV-2 BQ.1.1 variant-RBD
(GISAID accession number EPI_ISL_15370776: containing residues 331-524 of SARS-CoV-2 S
protein with G339D, R346T, S371F, S373P, S375F, T376A, D405N, R408S, K417N, N440K,
K444T, L452R, N460K, S477N, T478K, E484A, F486V, Q498R, N501Y, Y505H mutations). The
numbering was based on the S protein of original SARS-CoV-2 strain (GenBank accession no.
QHR63250.2: mutant residues, 339D, 346T, 371F, 373P, 375F, 376A, 405N, 408S, 417N, 440K,
444T, 452R, 460K, 477N, 478K, 484A, 486V, 498R, 501Y, and Y505H, as well as glycosylation
epitopes 519N and 521T, are underlined).
SEQ ID NO: 35
NITNLCPFDEVFNATTFASVYAWNRKRISNCVADYSVLYNFAPFFAFKCYGVSPTKLND
LCFTNVYADSFVIRGNEVSQIAPGQTGNIADYNYKLPDDFTGCVIAWNSNKLDSTVGGN
YNYRYRLFRKSKLKPFERDISTEIYQAGNKPCNGVAGVNCYFPLQSYGFRPTYGVGHQP
YRVVVLSFELLNATATV

### TABLES

Table 1 is a resources table for Example 3.

| | | |
|---|---|---|
| REAGENT or RESOURCE | SOURCE | IDENTIFIER |

Antibodies

| | | |
|---|---|---|
| Anti-mouse IgG-Fab-HRP | Sigma | A9917 |
| Mouse-anti-His-FITC | Thermo Fisher | MA1-81891 |
| | Scientific | |
| Anti-Mouse IgG1-HRP | Invitrogen | PA1-74421 |
| Anti-Mouse IgG2a-HRP | Invitrogen | M32207 |
| Anti-Mouse IgG2b-HRP | Invitrogen | M32407 |

Virus strains

| | | |
|---|---|---|
| Pseudotyped SARS-CoV-2 | GenBank | Accession No |
| original strain | | QHR63250.2 |
| Pseudotyped SARS-CoV-2 | GISAID | Accession No. |
| Alpha variant | | EPI_ISL_718813 |
| Pseudotyped SARS-CoV-2 | GISAID | Accession No. |
| Omicron BA1 subvariant | | EPI_ISL_6795835 |
| Pseudotyped SARS-CoV-2 | GISAID | Accession No. |
| Omicron BA2 subvariant | | EPI_ISL_12030355 |
| Pseudotyped SARS-CoV-2 | GISAID | Accession No. |
| Omicron BA2.12.1 | | EPI_ISL_12061569 |
| subvariant | | |
| Pseudotyped SARS-CoV-2 | GISAID | Accession No |
| Omicron BA5 subvariant | | EPI_ISL_12043290 |
| Live SARS-CoV-2 original | GenBank | Accession No |
| strain | | MN985325 |
| Live SARS-CoV-2 Delta | GISAID | Accession No |
| variant | | EPI_ISL_2331496 |

Chemicals

| | | |
|---|---|---|
| Agarose | Research Products | A20090-500 |
| | International | |
| Crystal violet | Fisher Scientific | C581-100 |
| Fat-free milk | Bio-Rad | 1706404 |
| Polyetherimide (PEI) | Sigma | 919012 |

Critical commercial assays

| | | |
|---|---|---|
| Cap 1 Capping System Kit | CELLSCRIPT | C-SCCS1710 |
| GenVoy-ILM | Precision | NWW0042 |
| | Nanosystems | |
| Luciferase Assay System | Promega | E1501 |
| MEGAscript T7 | Thermo Fisher | AMB1334-5 |
| Transcription Kit | Scientific | |
| Poly(A) Polymerase | CELLSCRIPT | C-PAP5104H |
| Tailing Kit | | |
| PNI Formulation Buffer | Precision | NWW0043 |
| | Nanosystems | |
| ToxinSensor ™ | GenScript | L00350 |
| Chromogenic | | |
| LAL Endotoxin Assay | | |

Experimental models: Cell lines

| | | |
|---|---|---|
| hACE2/293T | Laboratory stock | N/A |
| HEK293T | ATCC | CRL-3216 |
| Vero E6 | ATCC | CRL-1586 |

Recombinant DNA

| | | |
|---|---|---|
| pLenti-CMV-luciferase | Addgene | 17477 |
| PS-PAX2 | Addgene | 12260 |

Software and algorithms

| | | |
|---|---|---|
| FlowJo | BD Biosciences | N/A |
| Gen5 | BioTek Instruments | N/A |
| GraphPad Prism 9 | Graphpad Software | N/A |

Other

| | | |
|---|---|---|
| Fetal bovine serum (FBS) | R&D Systems | S11550 |
| Cell lysis buffer | Promega | E153A |
| Penicillin-Streptomycin | Thermo Fisher | 15140122 |
| (Gibco) | Scientific | |
| Pseudo-UTP | APExBIO | B7972 |
| Tetramethylbenzidine | Sigma | T0440 |
| (TMB) | | |
| Tween-20 | Sigma | P1379 |

What is claimed is:

1. A vaccine comprising an epitope from an original SARS-CoV-2 receptor-binding domain of a spike protein, wherein the epitope is glycosylated.

2. The vaccine of claim 1, wherein the receptor-binding domain comprises a glycosylation site at amino acid residue 519 and/or 521.

3. The vaccine of claim 1 or 2, wherein the epitope is from an Omicron variant of the original SARS-CoV-2.

4. The vaccine of claim 3, wherein the Omicron variant is selected from BA1, BA2, BA2.12.1, BA4, BA5, XBB1.5, XBB1.16, XBB1.9.1, XBB1.9.2, XBB2.3, other XBB, CH1.1, or BQ1.1.

5. A vaccine comprising an epitope from an original SARS-CoV-2 receptor-binding domain of a spike protein, wherein the epitope is glycosylated and fused to a Fc fragment of a human antibody.

6. The vaccine of claim 5, wherein the vaccine comprises a mucosal vaccine.

7. The vaccine of claim 5 or 6, wherein the receptor-binding domain comprises a glycosylation site at amino acid residues 519 and/or 521.

8. The vaccine of any one of claims 5-7, wherein the receptor-binding domain comprises a mutation at one or more amino acid residues selected from 371, 376, 405, 408, 452, or 486.

9. The vaccine of any one of claims 5-8, wherein the epitope is from a Delta variant of the original SARS-CoV-2.

10. The vaccine of any one of claims 5-9, wherein the human antibody comprises IgG.

11. A method of inducing an immune response against a SARS-CoV-2 virus, comprising administering the vaccine of any one of claims 1 to 10.

12. A method of preventing or treating a SARS-CoV-2 infection, comprising administering the vaccine of any one of claims 1 to 10.

13. A method of preventing or treating a SARS-CoV-2 infection, comprising:
administering a first SARS-CoV-2 vaccine comprising a first spike protein or a fragment thereof; and
administering a second SARS-CoV-2 vaccine comprising a second spike protein or a fragment thereof, wherein the second spike protein or a fragment thereof is from a BA1 spike (BA1-S) protein or other Omicron S protein (BA5, XBB1.5, XBB1.16, XBB1.91, XBB1.92, XBB2.3, CH1.1, or BQ1.1).

14. The method of claim 13, wherein the first spike protein or a fragment thereof is a wild-type spike (WT-S) protein or fragment thereof.

15. The method of claim 13 or 14, wherein the method produces a strong neutralizing antibody response.

16. The method of any one of claims 13-15, wherein the method produces a high-titer neutralizing antibody response against both the original SARS-CoV-2 and the Omicron subvariant (including BA1).

17. The method of any one of claims 13-16, wherein the first SARS-CoV-2 vaccine and the second SARS-CoV-2 vaccine are administered concurrently.

18. A method of preventing or treating a SARS-CoV-2 infection, comprising:
administering a subunit vaccine comprising a first spike protein or a fragment thereof; and
administering a booster dose comprising a second spike protein or a fragment thereof, wherein the second spike protein or a fragment thereof is from a BA1 spike (BA1-S) protein or other Omicron S protein (BA5, XBB1.5, XBB1.16, XBB1.91, XBB1.92, XBB2.3, CH1.1, or BQ1.1).

19. The method of claim 18, wherein the first spike protein or a fragment thereof is a wild-type spike (WT-S) protein or fragment thereof.

20. The method of claim 18 or 19, wherein the method produces a strong neutralizing antibody response.

21. The method of any one of claims 18-20, wherein the method produces a high-titer neutralizing antibody response against both the original SARS-CoV-2 and the Omicron subvariant.

22. An mRNA vaccine comprising a ribonucleic acid encoding:
a SARS-CoV-2 spike protein or fragment thereof, wherein the SARS-CoV-2 spike protein or fragment thereof comprises six proline amino acid substitutions and a mutated furin cleavage site; and
a folding protein.

23. The mRNA vaccine of claim 22, wherein the SARS-CoV-2 spike protein is from an Omicron variant of SARS-CoV-2.

24. The mRNA vaccine of claim 23, wherein the Omicron variant is a BA1 variant, or another Omicron variant (BA5, XBB1.5, XBB1.1.6, XBB1.91, XBB1.92, XBB2.3, CH1.1, or BQ1.1).

25. The mRNA vaccine of any one of claims 22-24, wherein the folding protein is a foldon protein.

26. The mRNA vaccine of any one of claims 22-25, wherein the vaccine further encodes a signal peptide.

27. The mRNA vaccine of claim 26, wherein the signal peptide is a tissue plasminogen activator (tPA) signal peptide.

28. The mRNA vaccine of any one of claim 22-27, wherein the vaccine further encodes a protein tag.

29. The mRNA vaccine of claim 28, wherein the protein tag is a polyhistidine tag comprising at least six histidine amino acids.

30. The mRNA vaccine of any one of claims 22-29, wherein the vaccine is encapsulated in a lipid nanoparticle.

31. A vaccine comprising a SARS-CoV-2 spike protein or fragment thereof, wherein the SARS-CoV-2 spike protein or fragment thereof is encoded from the ribonucleic acid of any one of claims 22-30.

32. A deoxyribonucleic acid (DNA) encoding the vaccine of any one of claims 22-31.

33. A method of preventing or treating a SARS-CoV-2 infection in a subject, the method comprising administering to the subject a composition comprising the vaccine of any one of claims 22-31.

34. The method of claim 33, wherein the method comprises administering to the subject a first booster comprising an mRNA vaccine encoding a receptor-binding domain (RBD) from an original SARS-CoV-2 spike (WT-S) protein, and administering to the subject a second booster comprising an mRNA vaccine encoding the RBD from the original SARS-CoV-2 spike (WT-S) protein.

35. The method of claim 33 or 34, wherein the composition, the first booster, and the second booster are administered sequentially.

36. The method of any one of claims 33-35, wherein the method comprises administering the composition on a first week.

37. The method of any one of claims 33-36, wherein the method comprises administering the first booster on a second, third or fourth week.

38. The method of any one of claims 33-37, wherein the method comprises administering the second booster on a seventh or eighth week.

39. The method of any one of claims 33-38, wherein the SARS-CoV-2 virus is an Alpha, Beta, Gamma, Delta, or Omicron SARS-CoV-2 variant, or subvariants thereof.

40. The method of any one of claims 33-39, wherein the method induces an immune response against a SARS-CoV-2 virus.

41. The method of claim 40, wherein the immune response comprises a high-titer neutralizing antibody response against the original SARS-CoV-2 wild-type (WT), Alpha, Beta, Gamma, Delta, or Omicron SARS-CoV-2 variants, or subvariants thereof.

42. The method of any one of claims 33-41, wherein the subject is a mammal.

43. The method of any one of claims 33-42, wherein the subject is a human.

44. A vaccine comprising:
a coronavirus spike protein backbone from a first coronavirus; and
a coronavirus spike protein receptor-binding domain (RBD) from a second coronavirus;
wherein the first coronavirus is a different coronavirus than the second coronavirus.

45. The vaccine of claim 44, wherein the first coronavirus is SARS-CoV-1 or MERS-CoV.

46. The vaccine of claim 44, wherein the second coronavirus variant is SARS-CoV-2.

47. The vaccine of claim 44, wherein the first coronavirus is SARS-CoV-2.

48. The vaccine of claim 44, wherein the first coronavirus is an Omicron variant.

49. The vaccine of claim 44, wherein the first coronavirus is a BA1, BA2, BA5, XBB1.5, XBB1.16, XBB1.9.1, XBB1.9.2, XBB2.3, CH1.1, BQ1.1, or other XBB variant.

50. The vaccine of claim 44, wherein the second coronavirus is a SARS-CoV-1 variant.

51. The vaccine of claim 44, wherein the second coronavirus variant is SARS-CoV-1 or MERS-CoV.

52. The vaccine of claim 44, wherein the first coronavirus is selected from SARS-CoV-2 wild-type (WT), Alpha, Beta, Gamma, Delta, or Omicron (Omicron-BA1, Omicron-BA2, Omicron-BA2.75, Omicron-BA4.6, Omicron-BA5, Omicron-XBB1.5, Omicron-XBB1.16, Omicron-XBB1.9.1, Omicron-XBB1.9.2, Omicron-XBB2.3, Omicron-CH1.1, BQ1.1, or other Omicron-XBB).

53. The vaccine of claim 44, wherein the second coronavirus is selected from SARS-CoV-2 wild-type (WT), Alpha, Beta, Gamma, Delta, or Omicron (Omicron-BA1, Omicron-BA2, Omicron-BA2.75, Omicron-BA4.6, Omicron-BA5, Omicron-XBB1.5, Omicron-XBB1.16, Omicron-XBB1.9.1, Omicron-XBB1.9.2, Omicron-XBB2.3, Omicron-CH1.1, other Omicron-XBB, or BQ1.1).

54. A vaccine comprising:
a SARS-CoV-2 spike protein backbone from a first SARS-CoV-2 variant; and
a SARS-CoV-2 spike protein receptor-binding domain (RBD) from a second SARS-CoV-2 variant;
wherein the first SARS-CoV-2 variant is a different variant than the second SARS-CoV-2 variant.

55. The vaccine of claim 54, wherein the first SARS-CoV-2 variant is an Omicron variant.

56. The vaccine of claim 54, wherein the second SARS-CoV-2 variant is a Delta variant.

57. The vaccine of claim 54, wherein the first SARS-CoV-2 variant is a Delta variant.

58. The vaccine of claim 54, wherein the second SARS-CoV-2 variant is an Omicron variant.

59. The vaccine of claim 54, wherein the first SARS-CoV-2 variant is selected from SARS-CoV-2 wild-type (WT), Alpha, Beta, Gamma, Delta, or Omicron (Omicron-BA1, Omicron-BA2, Omicron-BA2.75, Omicron-BA4.6, Omicron-BA5, Omicron-XBB1.5, Omicron-XBB1.16, Omicron-XBB1.9.1, Omicron-XBB1.9.2, Omicron-XBB2.3, Omicron-CH1.1, other Omicron-XBB, or BQ1.1).

60. The vaccine of claim 54, wherein the second SARS-CoV-2 variant is selected from SARS-CoV-2 wild-type (WT), Alpha, Beta, Gamma, Delta, or Omicron (Omicron-BA1, Omicron-BA2, Omicron-BA2.75, Omicron-BA4.6, Omicron-BA5, Omicron-XBB1.5, Omicron-XBB1.16, Omicron-XBB1.9.1, Omicron-XBB1.9.2, Omicron-XBB2.3, Omicron-CH1.1, other Omicron-XBB, or BQ1.1).

61. The vaccine of claim 44, comprising a S-6P-Delta-RBD vaccine.

62. The vaccine of claim 54, comprising a S-6P-Delta-RBD vaccine.

63. The vaccine of any one of claims 44-62, wherein the vaccine comprises a protein subunit vaccine.

64. The vaccine of any one of claims 44-63, wherein the vaccine comprises an mRNA vaccine.

65. A method of inducing an immune response against a coronavirus, comprising administering the vaccine of any one of claims 44-64.

66. A method of preventing or treating a coronavirus infection, comprising administering the vaccine of any one of claims 44-64.

67. The method of claim 65 or 66, further comprising administering an additional vaccine.

68. The method of claim 67, wherein the additional vaccine comprises a SARS-CoV-1, a MERS-CoV, or a SARS-CoV-2-glycan mutant protein vaccine.
